(19) United States
(12) Patent Application Publication
(10) Pub. No.: US 2025/0136699 A1
(43) Pub. Date: May 1, 2025

(54) ANTIBODIES SPECIFICALLY RECOGNIZING FCRN AND USES THEREOF

(71) Applicant: Jiangsu BioJeTay Biotechnology Co., Ltd., Wuxi City, Jiangsu (CN)

(72) Inventors: Lei Zhang, Beijing (CN); Shuai Zhang, Beijing (CN); Jie Huang, Beijing (CN); Ping Li, Beijing (CN)

(21) Appl. No.: 18/681,232

(22) PCT Filed: Aug. 12, 2022
(86) PCT No.: PCT/CN2022/111998

§ 371 Date: Feb. 5, 2024

(30) Foreign Application Priority Data
Aug. 13, 2021 (WO) PCT/CN2021/112372

(51) Int. Cl. C07K 16/28; A61K 39/00

(52) U.S. Cl. CPC C07K 16/283; A61K 2039/505; C07K 2317/24; C07K 2317/33; C07K 2317/76; C07K 2317/92; C07K 2317/94

(57) ABSTRACT

Provided are antibodies including antigen-binding fragments thereof that specifically recognizing the neonatal Fc receptor (FcRn). Also provided are methods of making and using these antibodies.

### SUBMISSION OF SEQUENCE LISTING ON ASCII TEXT FILE

The contents of the electronic sequence listing (file name: FcRn antibody.xml, date recorded: Aug. 9, 2022, size: 91 KB) is incorporated herein by reference in its entirety.

### FIELD OF THE APPLICATION

This application pertains to antibodies that specifically recognize the neonatal Fc receptor (FcRn), and methods of manufacture and uses thereof, including methods of treating autoimmune disorders and/or inflammatory diseases.

### BACKGROUND OF THE APPLICATION

The neonatal Fc receptor (FcRn) is a major histocompatibility complex (MHC) class I type molecule, which is a heterodimer consisting of an α-chain and p2-microglobulin, encoded by FCGRT and B2M, respectively. FcRn binds to, transports, and recycles immunoglobulin G (IgG) and albumin, thereby protecting them from lysosomal degradation. It was first discovered in the intestine of neonatal rats, where it confers passive humoral immunity to the neonate by transporting maternal IgG from ingested milk through the intestinal epithelium and into the newborn's bloodstream (Brambell F W. The transmission of immunity from mother to young and the catabolism of immunoglobulins. Lancet. 1966; 2:1087-93; Simister N E, et al. Isolation and characterization of an Fc receptor from neonatal rat small intestine. Eur J Immunol. 1985; 15:733-8). A similar mode of function was later discovered in the human placenta, where FcRn transfers maternal IgG to the fetus across the syncytiotrophoblasts (Leach J L, et al. Isolation from human placenta of the IgG transporter, FcRn, and localization to the syncytiotrophoblast: implications for maternal-fetal antibody transport. J Immunol. 1996; 157:3317-22).

FcRn is a key player in regulating the dynamic behavior, including distribution, transport, and persistence, of IgG antibodies throughout the body. The identification of these activities extends the role of this Fc receptor well beyond its original identification as the transporter of IgG from mother to young (hence the name ‘n’, for neonatal), prompting extensive analyses of the molecular and cellular properties of FcRn.

IgG residues that are critical for the binding of mouse or human IgG1 to FcRn include Ile253, His310, His435, His436 (mouse) or Tyr436 (human) that are located on the exposed loops at the CH2-CH3 domain interface of IgG1(Kim, J. K. et al. (1994) Localization of the site of the murine IgG1 molecule that is involved in binding to the murine intestinal Fc receptor. Eur. J. Immunol. 24, 2429-2434; Martin, W. L. et al. (2001) Crystal structure at 2.8 Å of an FcRn/heterodimeric Fc complex: mechanism of pH dependent binding. Mol. Cell 7, 867-877; Kim, J. K. et al. (1999) Mapping the site on human IgG for binding of the MHC class I-related receptor, FcRn. Eur. J. Immunol. 29, 2819-2825). The His residues of IgG interact with acidic residues of FcRn (Vaughn, D. E. et al. (1997) Identification of critical IgG binding epitopes on the neonatal Fc receptor. J. Mol. Biol. 274, 597-607). The interaction of the protonated imidazole side chain of His (pKa 6) with these acidic residues at pH 6.0 confers the characteristic pH-dependent binding (relatively high affinity at acidic pH, with very weak to negligible binding at pH 7.3-7.4) that is observed for the majority of IgGs (Raghavan, M. et al. (1995) Analysis of the pH dependence of the neonatal Fc receptor/immunoglobulin G interaction using antibody and receptor variants. Biochemistry 34, 14649-14657; Zhou, J. et al. (2003) Generation of mutated variants of the human form of the MHC class I-related receptor, FcRn, with increased affinity for mouse immunoglobulin G. J. Mol. Biol. 332, 901-913). FcRn is ubiquitously expressed in both parenchymal (endothelial, epithelial) and hematopoietic cells (Zhu, X. et al. (2001) MHC class I-related neonatal Fc receptor for IgG is functionally expressed in monocytes, intestinal macrophages, and dendritic cells. J. Immunol. 166, 3266-3276; Akilesh, S. et al. (2007) Neonatal FcR expression in bone marrow-derived cells functions to protect serum IgG from catabolism. J. Immunol. 179, 4580-4588; Perez-Montoyo, H. et al. (2009) Conditional deletion of the MHC Class I-related receptor, FcRn, reveals the sites of IgG homeostasis in mice. Proc. Natl. Acad. Sci. U.S.A 106, 2788-2793). The acidic pH in early endosomes (pH 6.0) is permissive for FcRn-IgG interactions, whereas the extracellular, near-neutral pH (pH 7.4) for most cell types enables efficient dissociation of IgG from FcRn during fusion of exocytic compartments with the plasma membrane.

FcRn contains a binding site for serum albumin that is distinct from its binding site for the Fc domain of IgG, due to ionic interactions between FcRn and IgG or albumin on opposite faces of the FcRn heavy chain (Chaudhury et al., 2006, Biochemistry 45:4983-4990). Like its binding to IgG, binding of FcRn to albumin is strongly pH-dependent, occurring at acidic pH (typically less than pH 6, and optimally at pH 5) but not at neutral pH. Like its role in protecting IgG from degradation, FcRn binding of albumin protects albumin from degradation and results in an extended serum half-life for albumin.

The role of FcRn as a global regulator of IgG provides opportunities for the use of FcRn inhibitors to reduce the levels of antibodies that cause symptoms in diseases such as autoimmune disorders or inflammatory diseases. The FcRn inhibitors bind to FcRn with higher affinity than naturally occurring IgGs in the pH range 6.0-7.4 (Vaccaro, C. et al. (2005) Engineering the Fc region of immunoglobulin G to modulate in vivo antibody levels. Nat. Biotechnol. 23, 1283-1288; Liu, L. et al. (2007) Amelioration of experimental autoimmune myasthenia gravis in rats by neonatal FcR blockade. J. Immunol. 178, 5390-5398; Low, S. C. and Mezo, A. R. (2009) Inhibitors of the FcRn:IgG protein-protein interaction. AAPS J. 11, 432-434). Consequently, they compete with endogenous antibodies for FcRn binding, driving such antibodies into degradative lysosomes. Importantly, clinical studies have demonstrated that greater than 50% reductions in pathogenic antibody levels in several autoimmune disorders can result in therapeutic benefit (Khosroshahi, A. et al. (2010) Rituximab therapy leads to rapid decline of serum IgG4 levels and prompt clinical improvement in IgG4-related systemic disease. Arthritis Rheum. 62, 1755-1762; Ahmed, A. R. et al. (2006) Treatment of pemphigus vulgaris with rituximab and intravenous immune globulin. N. Engl. J. Med. 355, 1772-1779), indicating that even partial reduction by FcRn inhibitors could be effective. A further application area is to use FcRn inhibitors to decrease background and improve contrast during diagnostic/theragnostic imaging with (radio)labeled antibodies. FcRn inhibition also offers an alternative to current strategies to reduce pathogenic antibody levels, such as plasmapheresis, intravenous immunoglobulin (IVIG) delivery, or B cell depletion, all of which can have undesirable side effects.

To date, the results from studies in non-human primates and clinical trials for several FcRn based inhibitors indicate that they induce significant and sustained decreases in endogenous IgG levels in healthy volunteers and have beneficial effects in the autoimmune disease myasthenia gravis (Nixon, A. E. et al. (2015) Fully human monoclonal antibody inhibitors of the neonatal Fc receptor reduce circulating IgG in non-human primates. Front. Immunol. 6, 176; Kiessling, P. et al. (2017) The FcRn inhibitor Rozanolixizumab reduces human serum IgG concentration: a randomized phase 1 study. Sci. Transl. Med. 9, eaan1208). Anti-FcRn antibodies have been described previously in WO2014/19727, WO2015/71330. However, there remains a need for improved anti-FcRn antibodies.

The disclosures of all publications, patents, patent applications and published patent applications referred to herein are hereby incorporated herein by reference in their entirety.

### BRIEF SUMMARY OF THE APPLICATION

In some embodiments, there is provided an isolated anti-FcRn antibody comprising: a V_{H} comprising an HC-CDR1, an HC-CDR2, and an HC-CDR3 of a V_{H} comprising the amino acid sequence of SEQ ID NO: 41; and a V_{L} comprising an LC-CDR1, an LC-CDR2, and an LC-CDR3 of a V_{L} comprising the amino acid sequence of SEQ ID NO: 52.

In some embodiments, there is provided an isolated anti-FcRn antibody comprising: a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 15, or a variant thereof comprising up to about 5 amino acid substitutions in the HC-CDRs; and a V_{L} comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 34, or a variant thereof comprising up to about 5 amino acid substitutions in the LC-CDRs.

In some embodiments, according to any one of the isolated anti-FcRn antibodies described herein, the isolated anti-FcRn antibody comprises: a V_{H} comprising the amino acid sequence of SEQ ID NO: 41, or a variant thereof having at least about 80% (such as at least about any one of 80%, 85%, 90%, 95%, 96%, 97%, 98%, or 99%) sequence identity to the amino acid sequence of SEQ ID NO: 41; and a V_{L} comprising the amino acid sequence of SEQ ID NO: 52, or a variant thereof having at least about 80% (such as at least about any one of 80%, 85%, 90%, 95%, 96%, 97%, 98%, or 99%) sequence identity to the amino acid sequence of SEQ ID NO: 52.

In some embodiments, there is provided an isolated anti-FcRn antibody comprising: a V_{H} comprising an HC-CDR1, an HC-CDR2, and an HC-CDR3 of a V_{H} comprising the amino acid sequence of SEQ ID NO: 42; and a V_{L} comprising an LC-CDR1, an LC-CDR2, and an LC-CDR3 of a V_{L} comprising the amino acid sequence of SEQ ID NO: 53.

In some embodiments, there is provided an isolated anti-FcRn antibody comprising: a V_{H} comprising an HC-CDR1 comprising the amino acid sequence of SEQ ID NO: 2, an HC-CDR2 comprising the amino acid sequence of SEQ ID NO: 8, and an HC-CDR3 comprising the amino acid sequence of SEQ ID NO: 16, or a variant thereof comprising up to about 5 amino acid substitutions in the HC-CDRs; and a V_{L} comprising an LC-CDR1 comprising the amino acid sequence of SEQ ID NO: 24, an LC-CDR2 comprising the amino acid sequence of SEQ ID NO: 29, and an LC-CDR3 comprising the amino acid sequence of SEQ ID NO: 35, or a variant thereof comprising up to about 5 amino acid substitutions in the LC-CDRs.

In some embodiments, according to any one of the isolated anti-FcRn antibodies described herein, the isolated anti-FcRn antibody comprises: a V_{H} comprising the amino acid sequence of SEQ ID NO: 42, or a variant thereof having at least about 80% (such as at least about any one of 80%, 85%, 90%, 95%, 96%, 97%, 98%, or 99%) sequence identity to the amino acid sequence of SEQ ID NO: 42; and a V_{L} comprising the amino acid sequence of SEQ ID NO: 53, or a variant thereof having at least about 80% (such as at least about any one of 80%, 85%, 90%, 95%, 96%, 97%, 98%, or 99%) sequence identity to the amino acid sequence of SEQ ID NO: 53.

In some embodiments, there is provided an isolated anti-FcRn antibody comprising: a V_{H} comprising an HC-CDR1, an HC-CDR2, and an HC-CDR3 of a V_{H} comprising the amino acid sequence of SEQ ID NO: 43; and a V_{L} comprising an LC-CDR1, an LC-CDR2, and an LC-CDR3 of a V_{L} comprising the amino acid sequence of SEQ ID NO: 54.

In some embodiments, there is provided an isolated anti-FcRn antibody comprising: a V_{H} comprising an HC-CDR1 comprising the amino acid sequence of SEQ ID NO: 3, an HC-CDR2 comprising the amino acid sequence of SEQ ID NO: 9, and an HC-CDR3 comprising the amino acid sequence of SEQ ID NO: 17, or a variant thereof comprising up to about 5 amino acid substitutions in the HC-CDRs; and a V_{L} comprising an LC-CDR1 comprising the amino acid sequence of SEQ ID NO: 25, an LC-CDR2 comprising the amino acid sequence of SEQ ID NO: 30, and an LC-CDR3 comprising the amino acid sequence of SEQ ID NO: 36, or a variant thereof comprising up to about 5 amino acid substitutions in the LC-CDRs.

In some embodiments, according to any one of the isolated anti-FcRn antibodies described herein, the isolated anti-FcRn antibody comprises: a V_{H} comprising the amino acid sequence of SEQ ID NO: 43, or a variant thereof having at least about 80% sequence identity to the amino acid sequence of SEQ ID NO: 43; and a V_{L} comprising the amino acid sequence of SEQ ID NO: 54, or a variant thereof having at least about 80% sequence identity to the amino acid sequence of SEQ ID NO: 54.

In some embodiments, there is provided an isolated anti-FcRn antibody comprising: a V_{H} comprising an HC-CDR1, an HC-CDR2, and an HC-CDR3 of a V_{H} comprising the amino acid sequence of SEQ ID NO: 44; and a V_{L} comprising an LC-CDR1, an LC-CDR2, and an LC-CDR3 of a V_{L} comprising the amino acid sequence of SEQ ID NO: 55.

In some embodiments, there is provided an isolated anti-FcRn antibody comprising: a V_{H} comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 18, or a variant thereof comprising up to about 5 amino acid substitutions in the HC-CDRs; and a V_{L} comprising an LC-CDR1 comprising the amino acid sequence of SEQ ID NO: 26, an LC-CDR2 comprising the amino acid sequence of SEQ ID NO: 31, and an LC-CDR3 comprising the amino acid sequence of SEQ ID NO: 37, or a variant thereof comprising up to about 5 amino acid substitutions in the LC-CDRs.

In some embodiments, according to any one of the isolated anti-FcRn antibodies described herein, the isolated anti-FcRn antibody comprises: a V_{H} comprising the amino acid sequence of SEQ ID NO: 44, or a variant thereof having at least about 80% (such as at least about any one of 80%, 85%, 90%, 95%, 96%, 97%, 98%, or 99%) sequence identity to the amino acid sequence of SEQ ID NO: 44; and a V_{L} comprising the amino acid sequence of SEQ ID NO: 55, or a variant thereof having at least about 80% (such as at least about any one of 80%, 85%, 90%, 95%, 96%, 97%, 98%, or 99%) sequence identity to the amino acid sequence of SEQ ID NO: 55.

In some embodiments, there is provided an isolated anti-FcRn antibody comprising: a V_{H} comprising an HC-CDR1, an HC-CDR2, and an HC-CDR3 of a V_{H} comprising the amino acid sequence of SEQ ID NO: 45; and a V_{L} comprising an LC-CDR1, an LC-CDR2, and an LC-CDR3 of a V_{L} comprising the amino acid sequence of SEQ ID NO: 56.

In some embodiments, there is provided an isolated anti-FcRn antibody comprising: a V_{H} comprising an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising an LC-CDR1 comprising the amino acid sequence of SEQ ID NO: 25, an LC-CDR2 comprising the amino acid sequence of SEQ ID NO: 32, and an LC-CDR3 comprising the amino acid sequence of SEQ ID NO: 38, or a variant thereof comprising up to about 5 amino acid substitutions in the LC-CDRs.

In some embodiments, according to any one of the isolated anti-FcRn antibodies described herein, the isolated anti-FcRn antibody comprises: a V_{H} comprising the amino acid sequence of SEQ ID NO: 45, or a variant thereof having at least about 80% (such as at least about any one of 80%, 85%, 90%, 95%, 96%, 97%, 98%, or 99%) sequence identity to the amino acid sequence of SEQ ID NO: 45; and a V_{L} comprising the amino acid sequence of SEQ ID NO: 56, or a variant thereof having at least about 80% (such as at least about any one of 80%, 85%, 90%, 95%, 96%, 97%, 98%, or 99%) sequence identity to the amino acid sequence of SEQ ID NO: 56.

In some embodiments, there is provided an isolated anti-FcRn antibody comprising: a V_{H} comprising an HC-CDR1, an HC-CDR2, and an HC-CDR3 of a V_{H} comprising the amino acid sequence of SEQ ID NO: 46; and a V_{L} comprising an LC-CDR1, an LC-CDR2, and an LC-CDR3 of a V_{L} comprising the amino acid sequence of SEQ ID NO: 57.

In some embodiments, there is provided an isolated anti-FcRn antibody comprising: a V_{H} comprising an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 38, or a variant thereof comprising up to about 5 amino acid substitutions in the LC-CDRs.

In some embodiments, according to any one of the isolated anti-FcRn antibodies described herein, the isolated anti-FcRn antibody comprises: a V_{H} comprising the amino acid sequence of SEQ ID NO: 46, or a variant thereof having at least about 80% (such as at least about any one of 80%, 85%, 90%, 95%, 96%, 97%, 98%, or 99%) sequence identity to the amino acid sequence of SEQ ID NO: 46; and a V_{L} comprising the amino acid sequence of SEQ ID NO: 57, or a variant thereof having at least about 80% (such as at least about any one of 80%, 85%, 90%, 95%, 96%, 97%, 98%, or 99%) sequence identity to the amino acid sequence of SEQ ID NO: 57.

In some embodiments, there is provided an isolated anti-FcRn antibody comprising: a V_{H} comprising an HC-CDR1, an HC-CDR2, and an HC-CDR3 of a V_{H} comprising the amino acid sequence of SEQ ID NO: 47; and a V_{L} comprising an LC-CDR1, an LC-CDR2, and an LC-CDR3 of a V_{L} comprising the amino acid sequence of SEQ ID NO: 58.

In some embodiments, there is provided an isolated anti-FcRn antibody comprising: a V_{H} comprising an HC-CDR1 comprising the amino acid sequence of SEQ ID NO: 5, an HC-CDR2 comprising the amino acid sequence of SEQ ID NO: 12, and an HC-CDR3 comprising the amino acid sequence of SEQ ID NO: 21, or a variant thereof comprising up to about 5 amino acid substitutions in the HC-CDRs; and a V_{L} comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39, or a variant thereof comprising up to about 5 amino acid substitutions in the LC-CDRs.

In some embodiments, according to any one of the isolated anti-FcRn antibodies described herein, the isolated anti-FcRn antibody comprises: a V_{H} comprising the amino acid sequence of SEQ ID NO: 47, or a variant thereof having at least about 80% (such as at least about any one of 80%, 85%, 90%, 95%, 96%, 97%, 98%, or 99%) sequence identity to the amino acid sequence of SEQ ID NO: 47; and a V_{L} comprising the amino acid sequence of SEQ ID NO: 58, or a variant thereof having at least about 80% (such as at least about any one of 80%, 85%, 90%, 95%, 96%, 97%, 98%, or 99%) sequence identity to the amino acid sequence of SEQ ID NO: 58.

In some embodiments, there is provided an isolated anti-FcRn antibody comprising: a V_{H} comprising an HC-CDR1, an HC-CDR2, and an HC-CDR3 of a V_{H} comprising the amino acid sequence of SEQ ID NO: 48; and a V_{L} comprising an LC-CDR1, an LC-CDR2, and an LC-CDR3 of a V_{L} comprising the amino acid sequence of SEQ ID NO: 59.

In some embodiments, there is provided an isolated anti-FcRn antibody comprising: a V_{H} comprising an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 21, or a variant thereof comprising up to about 5 amino acid substitutions in the HC-CDRs; and a V_{L} comprising an LC-CDR1 comprising the amino acid sequence of SEQ ID NO: 28, an LC-CDR2 comprising the amino acid sequence of SEQ ID NO: 33, and an LC-CDR3 comprising the amino acid sequence of SEQ ID NO: 40, or a variant thereof comprising up to about 5 amino acid substitutions in the LC-CDRs.

In some embodiments, according to any one of the isolated anti-FcRn antibodies described herein, the isolated anti-FcRn antibody comprises: a V_{H} comprising the amino acid sequence of SEQ ID NO: 48, or a variant thereof having at least about 80% (such as at least about any one of 80%, 85%, 90%, 95%, 96%, 97%, 98%, or 99%) sequence identity to the amino acid sequence of SEQ ID NO: 48; and a V_{L} comprising the amino acid sequence of SEQ ID NO: 59, or a variant thereof having at least about 80% (such as at least about any one of 80%, 85%, 90%, 95%, 96%, 97%, 98%, or 99%) sequence identity to the amino acid sequence of SEQ ID NO: 59.

In some embodiments, there is provided an isolated anti-FcRn antibody comprising: a V_{H} comprising an HC-CDR1, an HC-CDR2, and an HC-CDR3 of a V_{H} comprising the amino acid sequence of SEQ ID NO: 49; and a V_{L} comprising an LC-CDR1, an LC-CDR2, and an LC-CDR3 of a V_{L} comprising the amino acid sequence of SEQ ID NO: 58.

In some embodiments, there is provided an isolated anti-FcRn antibody comprising: a V_{H} comprising an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 22, or a variant thereof comprising up to about 5 amino acid substitutions in the HC-CDRs; and a V_{L} comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39, or a variant thereof comprising up to about 5 amino acid substitutions in the LC-CDRs.

In some embodiments, according to any one of the isolated anti-FcRn antibodies described herein, the isolated anti-FcRn antibody comprises: a V_{H} comprising the amino acid sequence of SEQ ID NO: 49, or a variant thereof having at least about 80% (such as at least about any one of 80%, 85%, 90%, 95%, 96%, 97%, 98%, or 99%) sequence identity to the amino acid sequence of SEQ ID NO: 49; and a V_{L} comprising the amino acid sequence of SEQ ID NO: 58, or a variant thereof having at least about 80% (such as at least about any one of 80%, 85%, 90%, 95%, 96%, 97%, 98%, or 99%) sequence identity to the amino acid sequence of SEQ ID NO: 58.

In some embodiments, there is provided an isolated anti-FcRn antibody comprising: a V_{H} comprising an HC-CDR1 comprising the amino acid sequence of SEQ ID NO: 1, or a variant thereof comprising up to about 3 amino acid substitutions; an HC-CDR2 comprising the amino acid sequence of SEQ ID NO: 7, or a variant thereof comprising up to about 3 amino acid substitutions; and an HC-CDR3 comprising the amino acid sequence of SEQ ID NO: 15, or a variant thereof comprising up to about 3 amino acid substitutions; and a V_{L} comprising an LC-CDR1 comprising the amino acid sequence of SEQ ID NO: 23, or a variant thereof comprising up to about 3 amino acid substitutions; an LC-CDR2 comprising the amino acid sequence of SEQ ID NO: 29, or a variant thereof comprising up to about 3 amino acid substitutions; and an LC-CDR3 comprising the amino acid sequence of SEQ ID NO: 34, or a variant thereof comprising up to about 3 amino acid substitutions.

In some embodiments, there is provided an isolated anti-FcRn antibody comprising a V_{H} comprising an HC-CDR1, an HC-CDR2, and an HC-CDR3 of a V_{H} comprising the amino acid sequence of any one of SEQ ID NOs: 50-51; and a V_{L} comprising an LC-CDR1, an LC-CDR2, and an LC-CDR3 of a V_{L} comprising the amino acid sequence of any one of SEQ ID NOs: 60-61.

In some embodiments, there is provided an isolated anti-FcRn antibody comprising: (i) a V_{H} comprising an HC-CDR1, an HC-CDR2, and an HC-CDR3 of a V_{H} comprising the amino acid sequence of SEQ ID NO: 50; and a V_{L} comprising an LC-CDR1, an LC-CDR2, and an LC-CDR3 of a V_{L} comprising the amino acid sequence of SEQ ID NO: 60; (ii) a V_{H} comprising an HC-CDR1, an HC-CDR2, and an HC-CDR3 of a V_{H} comprising the amino acid sequence of SEQ ID NO: 51; and a V_{L} comprising an LC-CDR1, an LC-CDR2, and an LC-CDR3 of a V_{L} comprising the amino acid sequence of SEQ ID NO: 61; (iii) a V_{H} comprising an HC-CDR1, an HC-CDR2, and an HC-CDR3 of a V_{H} comprising the amino acid sequence of SEQ ID NO: 50; and a V_{L} comprising an LC-CDR1, an LC-CDR2, and an LC-CDR3 of a V_{L} comprising the amino acid sequence of SEQ ID NO: 61.

In some embodiments, according to any one of the isolated anti-FcRn antibodies described herein, the isolated anti-FcRn antibody comprises: a V_{H} comprising the amino acid sequence of any one of SEQ ID NOs: 50-51, or a variant thereof having at least about 80% (such as at least about any one of 80%, 85%, 90%, 95%, 96%, 97%, 98%, or 99%) sequence identity to the amino acid sequence of any one of SEQ ID NOs: 50-51; and a V_{L} comprising the amino acid sequence of any one of SEQ ID NOs: 60-61, or a variant thereof having at least about 80% (such as at least about any one of 80%, 85%, 90%, 95%, 96%, 97%, 98%, or 99%) sequence identity to the amino acid sequence of any one of SEQ ID NOs: 60-61.

In some embodiments, the isolated anti-FcRn antibody comprises: (i) a V_{H} comprising the amino acid sequence of SEQ ID NO: 50, or a variant thereof having at least about 80% (such as at least about any one of 80%, 85%, 90%, 95%, 96%, 97%, 98%, or 99%) sequence identity to the amino acid sequence of SEQ ID NO: 50; and a V_{L} comprising the amino acid sequence of SEQ ID NO: 60, or a variant thereof having at least about 80% (such as at least about any one of 80%, 85%, 90%, 95%, 96%, 97%, 98%, or 99%) sequence identity to the amino acid sequence of SEQ ID NO: 60; (ii) a V_{H} comprising the amino acid sequence of SEQ ID NO: 51, or a variant thereof having at least about 80% (such as at least about any one of 80%, 85%, 90%, 95%, 96%, 97%, 98%, or 99%) sequence identity to the amino acid sequence of SEQ ID NO: 51; and a V_{L} comprising the amino acid sequence of SEQ ID NO: 61, or a variant thereof having at least about 80% (such as at least about any one of 80%, 85%, 90%, 95%, 96%, 97%, 98%, or 99%) sequence identity to the amino acid sequence of SEQ ID NO: 61; (iii) a V_{H} comprising the amino acid sequence of SEQ ID NO: 50, or a variant thereof having at least about 80% (such as at least about any one of 80%, 85%, 90%, 95%, 96%, 97%, 98%, or 99%) sequence identity to the amino acid sequence of SEQ ID NO: 50; and a V_{L} comprising the amino acid sequence of SEQ ID NO: 61, or a variant thereof having at least about 80% (such as at least about any one of 80%, 85%, 90%, 95%, 96%, 97%, 98%, or 99%) sequence identity to the amino acid sequence of SEQ ID NO: 61.

In some embodiments, there is provided an isolated anti-FcRn antibody that specifically binds to the human FcRn with a Kd from about 0.1 pM to about 1 nM.

In some embodiments, there is provided an isolated anti-FcRn antibody that specifically binds to FcRn competitively with any one of the isolated anti-FcRn antibodies described herein. In some embodiments, there is provided an isolated anti-FcRn antibody that specifically binds to the same epitope as any one of isolated anti-FcRn antibodies described herein.

In some embodiments according to any of the isolated anti-FcRn antibodies described herein, the isolated anti-FcRn antibody comprises an Fc fragment. In some embodiments, the isolated anti-FcRn antibody is a full-length IgG antibody. In some embodiments, the isolated anti-FcRn antibody is a full-length IgG1, IgG2, IgG3, or IgG4 antibody. In some embodiments, the anti-FcRn antibody is chimeric, human, or humanized antibody. In some embodiments, the anti-FcRn antibody is an antigen binding fragment selected from the group consisting of a Fab, a Fab′, a F(ab)′₂, a Fab′-SH, a single-chain Fv (scFv), an Fv fragment, a dAb, a Fd, a nanobody, a diabody, and a linear antibody.

In some embodiments, there is provided isolated nucleic acid molecule(s) that encodes any one of the anti-FcRn antibodies described herein. In some embodiments, there is provided a vector comprising any one of the nucleic acid molecules described herein. In some embodiments, there is provided a host cell comprising any one of the anti-FcRn antibodies described herein, any one of the nucleic acid molecules described herein, or any one of the vectors described herein. In some embodiments, there is provided a method of producing an anti-FcRn antibody, comprising: a) culturing any one of the host cells described herein under conditions effective to express the anti-FcRn antibody; and b) obtaining the expressed anti-FcRn antibody from the host cell.

In some embodiments, there is provided a method of treating a disease or condition in an individual in need thereof, comprising administering to the individual an effective amount of any one of the anti-FcRn antibody described herein. In some embodiments, there is provided the use of any one of the anti-FcRn antibodies described herein for the preparation of pharmaceutical compositions for treating a disease or condition in an individual in need. In some embodiments, provided is the use of the anti-FcRn antibodies described herein, or a pharmaceutical composition comprising an anti-FcRn antibodies described herein in the manufacture of a medicament for treating a disease or condition. In some embodiments, the disease or condition, comprise autoimmune disorders and inflammatory diseases. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome.

Also provided are pharmaceutical compositions, kits and articles of manufacture comprising any one of the anti-FcRn antibodies described herein.

### BRIEF DESCRIPTION OF THE DRAWINGS

FIG. 1 shows the binding affinity of exemplary chimeric anti-FcRn antibodies to human FcRn as analyzed by ELISA.

FIGS. 2A-2B show the binding affinity of exemplary chimeric anti-FcRn antibodies to human FCGRT as analyzed by ELISA. FIG. 2C shows that the chimeric anti-FcRn antibodies do not bind to human B2M.

FIGS. 3A-3C show the binding cross-reactivity to FcRn from different species. FIG. 3A shows the binding affinity of exemplary chimeric anti-FcRn antibodies to cynoFcRn. FIG. 3B shows that exemplary chimeric anti-FcRn antibodies do not bind to rFcRn, and FIG. 3C shows weak binding to mFcRn.

FIG. 4 shows exemplary chimeric anti-FcRn antibodies that exhibit a high ability to block human IgG binding to human FcRn.

FIG. 5 shows exemplary chimeric anti-FcRn antibodies that effectively inhibit the recycling of human IgG.

FIG. 6A shows the binding ability of humanized anti-FcRn antibodies to human FcRn as analyzed by ELISA. FIG. 6B shows the binding ability of humanized anti-FcRn antibodies to cynoFcRn as analyzed by ELISA.

FIG. 7A shows the binding affinity of humanized anti-FcRn antibodies to human FCGRT as analyzed by ELISA. FIG. 7B shows that humanized anti-FcRn antibodies do not bind to human B2M.

FIG. 8 shows that the humanized anti-FcRn antibodies effectively compete with human IgG and inhibit the binding of human IgG to human FcRn.

FIG. 9 shows that the humanized anti-FcRn antibodies effectively inhibit the recycling of human IgG.

FIG. 10 shows that the humanized anti-FcRn antibodies do not inhibit the binding of HSA to FcRn.

FIG. 11 shows the pharmacodynamics assay results of the anti-FcRn antibodies ZLP193, ZLP1-3-2M, and ZLP1-3-2F as compared to the reference antibody Rozanolixizumab.

### DETAILED DESCRIPTION OF THE APPLICATION

The present application in one aspect provides an isolated anti-FcRn antibody that specifically binds to human and/or cynomolgus monkey FcRn. By using a combination of the immunization of animals, selections on scFv phage libraries, and appropriately designed biochemical and biological assays, we have identified highly potent antibody molecules that bind to human and/or cynomolgus monkey FcRn and inhibit the binding of human IgG to FcRn. The results presented herein indicate that our antibodies bind human and/or cynomolgus monkey FcRn with high affinity, and surprisingly are even more potent than Rozanolixizumab (UCB, anti-FcRn antibody) as demonstrated in a variety of biological assays.

The anti-FcRn antibodies provided by the present application include, for example, full-length anti-FcRn antibodies, anti-FcRn scFvs, anti-FcRn Fc fusion proteins, multi-specific (such as bispecific) anti-FcRn antibodies, anti-FcRn immunoconjugates, and the like.

Also provided are nucleic acids encoding the anti-FcRn antibodies, compositions comprising the anti-FcRn antibodies, and methods of making and using the anti-FcRn antibodies.

### Definitions

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of the disease (such as, for example, tumor volume for cancer). The methods of the application contemplate any one or more of these aspects of treatment.

The term “antibody” includes full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein includes an antibody fragment including, for example, a diabody, a Fab, a Fab′, an F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a single domain antibody, a nanobody, a domain antibody, a bivalent domain antibody, or any other antibody fragments that bind to an antigen but do not comprise a complete antibody structure. An antigen-binding fragment also includes a fusion protein that comprises the antibody fragment described above. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen. As used herein, a first antibody “competes” for binding to a target FcRn with a second antibody when the first antibody inhibits the target FcRn binding of the second antibody by at least about 50% (such as at least about any of 55%, 60%, 65%, 70%, 75%, 80%, 85%, 90%, 95%, 98% or 99%) in the presence of an equimolar concentration of the first antibody, or vice versa. A high throughput process for “binning” antibodies based upon their cross-competition is described in PCT Publication No. WO 03/48731.

As used herein, the term “specifically binds”, “specifically recognizing”, or “is specific for” refers to measurable and reproducible interactions, such as binding between a target and an antibody that is determinative of the presence of the target in the presence of a heterogeneous population of molecules, including biological molecules. For example, an antibody that specifically recognizes a target (which can be an epitope) is an antibody that binds to this target with greater affinity, avidity, more readily, and/or with greater duration than its bindings to other targets. In some embodiments, an antibody that specifically recognizes an antigen reacts with one or more antigenic determinants of the antigen with a binding affinity that is at least about 10 times its binding affinity for other targets.

An “isolated” anti-FcRn antibody as used herein refers to an anti-FcRn antibody that (1) is not associated with proteins found in nature, (2) is free of other proteins from the same source, (3) is expressed by a cell from a different species, or, (4) does not occur in nature.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The term “chimeric antibody” refers to an antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this application (see U.S. Pat. No. 4,816,567; and Morrison et al., Proc. Natl. Acad. Sci. USA, 81:6851-6855 (1984)).

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv”, also abbreviated as “sFv” or “scFv”, are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skilled in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

The terms “Fc receptor” or “FcR” are used to describe a receptor that binds to the Fc region of an antibody. In some embodiments, an FcR of this application is one that binds to an IgG antibody (a γ receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daeron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “FcRn” refers to the neonatal Fc receptor (FcRn). FcRn is structurally similar to major histocompatibility complex (MHC) and consists of an α-chain noncovalently bound to p2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

The “CH1 domain” of a human IgG Fc region usually extends from about amino acid 118 to about amino acid 215 (EU numbering system).

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” comprises the stretch of residues of the C-terminal to a CH2 domain in an Fc region (i.e. from about amino acid residue 341 to the C-terminal end of an antibody sequence, typically at amino acid residue 446 or 447 of an IgG).

A “functional Fc fragment” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an antibody variable domain) and can be assessed using various assays known in the art.

An antibody with a variant IgG Fc with “altered” FcR binding affinity or ADCC activity is one which has either enhanced or diminished FcR binding activity (e.g., FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent Kd or IC₅₀ value) than the parent polypeptide or a native sequence IgG Fc. According to some embodiments, the improvement in binding compared to a parent polypeptide is about 3-fold, such as about any of 5, 10, 25, 50, 60, 100, 150, 200, or up to 500-fold, or about 25% to 1000% improvement in binding. The polypeptide variant which “exhibits decreased binding” to an FcR, binds at least one FcR with lower affinity (e.g., higher apparent Kd or IC₅₀ value) than a parent polypeptide. The decrease in binding compared to a parent polypeptide may be about 40% or more decrease in binding.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The polypeptide comprising a variant Fc region which “exhibits increased ADCC” or mediates ADCC in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using any in vitro ADCC assay known in the art, such as assays or methods for determining ADCC activity, e.g., in an animal model etc. In some embodiments, the variant is from about 5-fold to about 100-fold, e.g. from about 25 to about 50 fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed. Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See also, Idusogie et al. J. Immunol. 164: 4178-4184 (2000).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position.

The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

An “effective amount” of an anti-FcRn antibody or composition as disclosed herein, is an amount sufficient to carry out a specifically stated purpose. An “effective amount” can be determined empirically and by known methods relating to the stated purpose.

The term “therapeutically effective amount” refers to an amount of an anti-FcRn antibody or composition as disclosed herein, effective to treat, alleviate, or prevent a disease or disorder, or to manifest a detectable therapeutic or prophylactic effect in an individual. In the case of autoimmune disorders, the therapeutically effective amount of the anti-FcRn antibody or composition as disclosed herein can reduce the binding of IgG Fc to FcRn; reduce the half-life of circulating IgG; reduce the serum IgG concentration; inhibit (i.e., slow to some extent and preferably stop) the reaction between subject's autoantibodies and host tissue; inhibit immune effector T cells reactive with endogenous self-peptides, and result in tissue destruction. In some embodiments, the therapeutically effective amount is an FcRn blocking amount that induces overall pathogenic antibody catabolism and multiple pathogenic antibody removal. In some embodiments, the therapeutically effective amount is an amount that extends the survival of a patient. In some embodiments, the therapeutically effective amount is an amount that improves progression free survival of a patient.

As used herein, by “pharmaceutically acceptable” or “pharmacologically compatible” is meant a material that is not biological or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a”, “an”, and “the” include plural referents unless the context clearly dictates otherwise.

### Anti-FcRn Antibodies

In one aspect, the present application provides anti-FcRn antibodies that specifically bind to human and/or cynomolgus monkey FcRn. In one aspect, the present application provides anti-FcRn antibodies that weakly bind to mouse FcRn. Anti-FcRn antibodies include, but are not limited to, humanized antibodies, chimeric antibodies, mouse antibodies, human antibodies, and antibodies comprising the heavy chain and/or light chain CDRs discussed herein. In one aspect, the present application provides isolated antibodies that bind to FcRn. Contemplated anti-FcRn antibodies include, for example, full-length anti-FcRn antibodies (e.g., full-length IgG1 or IgG4), anti-FcRn scFvs, anti-FcRn Fc fusion proteins, multi-specific (such as bispecific) anti-FcRn antibodies, anti-FcRn immunoconjugates, and the like. In some embodiments, the anti-FcRn antibody is a full-length antibody (e.g., full-length IgG1 or IgG4) or antigen-binding fragment thereof, which specifically binds to FcRn. In some embodiments, the anti-FcRn antibody is a Fab, a Fab′, a F(ab)′₂, a Fab′-SH, a single-chain Fv (scFv), an Fv fragment, a dAb, a Fd, a nanobody, a diabody, or a linear antibody. In some embodiments, reference to an antibody that specifically binds to FcRn means that the antibody binds to FcRn with an affinity that is at least about 10 times (including for example at least about any one of 10, 10², 10³, 10⁴, 10⁵, 10⁶, or 10⁷ times) more tightly than its binding affinity for a non-target. In some embodiments, the non-target is an antigen that is not FcRn. Binding affinity can be determined by methods known in the art, such as ELISA, fluorescence activated cell sorting (FACS) analysis, or radioimmunoprecipitation assay (RIA). Kd can be determined by methods known in the art, such as surface plasmon resonance (SPR) assay or biolayer interferometry (BLI).

Although anti-FcRn antibodies containing human sequences (e.g., human heavy and light chain variable domain sequences comprising human CDR sequences) are extensively discussed herein, non-human anti-FcRn antibodies are also contemplated. In some embodiments, non-human anti-FcRn antibodies comprise human CDR sequences from an anti-FcRn antibody as described herein and non-human framework sequences. Non-human framework sequences include, in some embodiments, any sequence that can be used for generating synthetic heavy and/or light chain variable domains using one or more human CDR sequences as described herein, including, e.g., mammals, e.g., mouse, rat, rabbit, pig, bovine (e.g., cow, bull, buffalo), deer, sheep, goat, chicken, cat, dog, ferret, primate (e.g., marmoset, rhesus monkey), etc. In some embodiments, a non-human anti-FcRn antibody includes an anti-FcRn antibody generated by grafting one or more human CDR sequences as described herein onto a non-human framework sequence (e.g., a mouse or chicken framework sequence).

The amino acid sequence of an exemplary native human FcRn α-chain (FCGRT) comprises or consists of the amino acid sequence of SEQ ID NO: 66, the human FcRn 02-microglobulin (B2M) comprises or consists of the amino acid sequence of SEQ ID NO: 67.

In some embodiments, the anti-FcRn antibody described herein specifically recognizes an epitope within human FcRn. In some embodiments, the anti-FcRn antibody cross-reacts with FcRn from species other than Homo sapiens. In some embodiments, the anti-FcRn antibody cross-reacts with FcRn from cynomolgus monkey. In some embodiments, the anti-FcRn antibody is completely specific for human FcRn and does not exhibit cross-reactivity with non-human species or other types of FcRn.

In some embodiments, the anti-FcRn antibody cross-reacts with at least one allelic variant of the FcRn protein (or fragments thereof). In some embodiments, the allelic variant has up to about 30 (such as about any of 1, 2, 3, 4, 5, 6, 7, 8, 9, 10, 15, 20, 25, or 30) amino acid substitutions (such as a conservative substitution) when compared to the naturally occurring FcRn (or fragments thereof). In some embodiments, the anti-FcRn antibody does not cross-react with any allelic variant of the FcRn protein (or fragments thereof).

In some embodiments, the anti-FcRn antibody cross-reacts with at least one interspecies variant of the FcRn protein. In some embodiments, for example, the FcRn protein (or fragments thereof) is human FcRn and the interspecies variant of the FcRn protein (or fragments thereof) is a cynomolgus monkey variant thereof. In some embodiments, the anti-FcRn antibody does not cross-react with any interspecies variant of the FcRn protein.

In some embodiments, according to any of the anti-FcRn antibodies described herein, the anti-FcRn antibody comprises an antibody heavy chain constant region and an antibody light chain constant region. In some embodiments, the anti-FcRn antibody comprises an IgG1 heavy chain constant region. In some embodiments, the anti-FcRn antibody comprises an IgG2 heavy chain constant region. In some embodiments, the anti-FcRn antibody comprises an IgG3 heavy chain constant region. In some embodiments, the anti-FcRn antibody comprises an IgG4 heavy chain constant region. In some embodiments, the heavy chain constant region comprises (including consisting of or consisting essentially of) the amino acid sequence of SEQ ID NO: 62. In some embodiments, the heavy chain constant region comprises (including consisting of or consisting essentially of) the amino acid sequence of SEQ ID NO: 63. In some embodiments, the anti-FcRn antibody comprises a kappa light chain constant region. In some embodiments, the light chain constant region comprises (including consisting of or consisting essentially of) the amino acid sequence of SEQ ID NO: 64. In some embodiments, the anti-FcRn antibody comprises a lambda light chain constant region. In some embodiments, the light chain constant region comprises (including consisting of or consisting essentially of) the amino acid sequence of SEQ ID NO: 65. In some embodiments, the anti-FcRn antibody comprises an antibody heavy chain variable domain and an antibody light chain variable domain.

In some embodiments, the anti-FcRn antibody comprises a V_{H} comprising: an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 15, or a variant thereof comprising up to about 5 amino acid substitutions in the HC-CDRs; and a V_{L} comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 34, or a variant thereof comprising up to about 5 amino acid substitutions in the LC-CDRs.

In some embodiments, the anti-FcRn antibody comprises a V_{H} comprising: an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 15; and a V_{L} comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 34.

In some embodiments, the anti-FcRn antibody comprises a V_{H} comprising the amino acid sequences of SEQ ID NOs: 1, 7 and 15, or a variant thereof comprising up to about 5 amino acid substitutions; and a V_{L} comprising the amino acid sequences of SEQ ID NOs: 23, 29 and 34, or a variant thereof comprising up to about 5 amino acid substitutions. In some embodiments, the anti-FcRn antibody comprises a V_{H} comprising the amino acid sequences of SEQ ID NOs: 1, 7 and 15; and a V_{L} comprising the amino acid sequences of SEQ ID NOs: 23, 29 and 34.

In some embodiments, the anti-FcRn antibody comprises a V_{H} comprising an HC-CDR1, an HC-CDR2 and an HC-CDR3 of the V_{H} comprising the amino acid sequence of SEQ ID NO: 41; and a V_{L} comprising an LC-CDR1, an LC-CDR2, and an LC-CDR3 of the V_{L} comprising the amino acid sequence of SEQ ID NO: 52.

In some embodiments, the anti-FcRn antibody comprises a V_{H} comprising one, two or three HC-CDRs of SEQ ID NO: 41.

In some embodiments, the anti-FcRn antibody comprises a V_{L} comprising one, two or three LC-CDRs of SEQ ID NO: 52.

In some embodiments, the anti-FcRn antibody comprises a V_{H} comprising HC-CDR1, HC-CDR2 and HC-CDR3 of the V_{H} of SEQ ID NO: 41, and a V_{L} comprising LC-CDR1, LC-CDR2 and LC-CDR3 of the V_{L} of SEQ ID NO: 52.

In some embodiments, the anti-FcRn antibody comprises a V_{H} comprising the amino acid sequence of SEQ ID NO: 41, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 52, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 41 and a V_{L} comprising the amino acid sequence of SEQ ID NO: 52.

In some embodiments, the anti-FcRn antibody comprises a V_{H} comprising: an HC-CDR1 comprising the amino acid sequence of SEQ ID NO: 2, an HC-CDR2 comprising the amino acid sequence of SEQ ID NO: 8, and an HC-CDR3 comprising the amino acid sequence of SEQ ID NO: 16, or a variant thereof comprising up to about 5 amino acid substitutions in the HC-CDRs; and a V_{L} comprising an LC-CDR1 comprising the amino acid sequence of SEQ ID NO: 24, an LC-CDR2 comprising the amino acid sequence of SEQ ID NO: 29, and an LC-CDR3 comprising the amino acid sequence of SEQ ID NO: 35, or a variant thereof comprising up to about 5 amino acid substitutions in the LC-CDRs.

In some embodiments, the anti-FcRn antibody comprises a V_{H} comprising: an HC-CDR1 comprising the amino acid sequence of SEQ ID NO: 2, an HC-CDR2 comprising the amino acid sequence of SEQ ID NO: 8, and an HC-CDR3 comprising the amino acid sequence of SEQ ID NO: 16; and a V_{L} comprising an LC-CDR1 comprising the amino acid sequence of SEQ ID NO: 24, an LC-CDR2 comprising the amino acid sequence of SEQ ID NO: 29, and an LC-CDR3 comprising the amino acid sequence of SEQ ID NO: 35.

In some embodiments, the anti-FcRn antibody comprises a V_{H} comprising the amino acid sequences of SEQ ID NOs: 2, 8 and 16, or a variant thereof comprising up to about 5 amino acid substitutions; and a V_{L} comprising the amino acid sequences of SEQ ID NOs: 24, 29 and 35, or a variant thereof comprising up to about 5 amino acid substitutions. In some embodiments, the anti-FcRn antibody comprises a V_{H} comprising the amino acid sequences of SEQ ID NOs: 2, 8 and 16; and a V_{L} comprising the amino acid sequences of SEQ ID NOs: 24, 29 and 35.

In some embodiments, the anti-FcRn antibody comprises a V_{H} comprising an HC-CDR1, an HC-CDR2 and an HC-CDR3 of the V_{H} comprising the amino acid sequence of SEQ ID NO: 42; and a V_{L} comprising an LC-CDR1, an LC-CDR2, and an LC-CDR3 of the V_{L} comprising the amino acid sequence of SEQ ID NO: 53.

In some embodiments, the anti-FcRn antibody comprises a V_{H} comprising one, two or three HC-CDRs of SEQ ID NO: 42.

In some embodiments, the anti-FcRn antibody comprises a V_{L} comprising one, two or three LC-CDRs of SEQ ID NO: 53.

In some embodiments, the anti-FcRn antibody comprises a V_{H} comprising HC-CDR1, HC-CDR2 and HC-CDR3 of the V_{H} of SEQ ID NO: 42, and a V_{L} comprising LC-CDR1, LC-CDR2 and LC-CDR3 of the V_{L} of SEQ ID NO: 53.

In some embodiments, the anti-FcRn antibody comprises a V_{H} comprising the amino acid sequence of SEQ ID NO: 42, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 53, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 42 and a V_{L} comprising the amino acid sequence of SEQ ID NO: 53.

In some embodiments, the anti-FcRn antibody comprises a V_{H} comprising: an HC-CDR1 comprising the amino acid sequence of SEQ ID NO: 3, an HC-CDR2 comprising the amino acid sequence of SEQ ID NO: 9, and an HC-CDR3 comprising the amino acid sequence of SEQ ID NO: 17, or a variant thereof comprising up to about 5 amino acid substitutions in the HC-CDRs; and a V_{L} comprising an LC-CDR1 comprising the amino acid sequence of SEQ ID NO: 25, an LC-CDR2 comprising the amino acid sequence of SEQ ID NO: 30, and an LC-CDR3 comprising the amino acid sequence of SEQ ID NO: 36, or a variant thereof comprising up to about 5 amino acid substitutions in the LC-CDRs.

In some embodiments, the anti-FcRn antibody comprises a V_{H} comprising: an HC-CDR1 comprising the amino acid sequence of SEQ ID NO: 3, an HC-CDR2 comprising the amino acid sequence of SEQ ID NO: 9, and an HC-CDR3 comprising the amino acid sequence of SEQ ID NO: 17; and a V_{L} comprising an LC-CDR1 comprising the amino acid sequence of SEQ ID NO: 25, an LC-CDR2 comprising the amino acid sequence of SEQ ID NO: 30, and an LC-CDR3 comprising the amino acid sequence of SEQ ID NO: 36.

In some embodiments, the anti-FcRn antibody comprises a V_{H} comprising the amino acid sequences of SEQ ID NOs: 3, 9 and 17, or a variant thereof comprising up to about 5 amino acid substitutions; and a V_{L} comprising the amino acid sequences of SEQ ID NOs: 25, 30 and 36, or a variant thereof comprising up to about 5 amino acid substitutions. In some embodiments, the anti-FcRn antibody comprises a V_{H} comprising the amino acid sequences of SEQ ID NOs: 3, 9 and 17; and a V_{L} comprising the amino acid sequences of SEQ ID NOs: 25, 30 and 36.

In some embodiments, the anti-FcRn antibody comprises a V_{H} comprising an HC-CDR1, an HC-CDR2 and an HC-CDR3 of the V_{H} comprising the amino acid sequence of SEQ ID NO: 43; and a V_{L} comprising an LC-CDR1, an LC-CDR2, and an LC-CDR3 of the V_{L} comprising the amino acid sequence of SEQ ID NO: 54.

In some embodiments, the anti-FcRn antibody comprises a V_{H} comprising one, two or three HC-CDRs of SEQ ID NO: 43.

In some embodiments, the anti-FcRn antibody comprises a V_{L} comprising one, two or three LC-CDRs of SEQ ID NO: 54.

In some embodiments, the anti-FcRn antibody comprises a V_{H} comprising HC-CDR1, HC-CDR2 and HC-CDR3 of the V_{H} of SEQ ID NO: 43, and a V_{L} comprising LC-CDR1, LC-CDR2 and LC-CDR3 of the V_{L} of SEQ ID NO: 54.

In some embodiments, the anti-FcRn antibody comprises a V_{H} comprising the amino acid sequence of SEQ ID NO: 43, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 54, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 43 and a V_{L} comprising the amino acid sequence of SEQ ID NO: 54.

In some embodiments, the anti-FcRn antibody comprises a V_{H} comprising: an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 18, or a variant thereof comprising up to about 5 amino acid substitutions in the HC-CDRs; and a V_{L} comprising an LC-CDR1 comprising the amino acid sequence of SEQ ID NO: 26, an LC-CDR2 comprising the amino acid sequence of SEQ ID NO: 31, and an LC-CDR3 comprising the amino acid sequence of SEQ ID NO: 37, or a variant thereof comprising up to about 5 amino acid substitutions in the LC-CDRs.

In some embodiments, the anti-FcRn antibody comprises a V_{H} comprising: an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 18; and a V_{L} comprising an LC-CDR1 comprising the amino acid sequence of SEQ ID NO: 26, an LC-CDR2 comprising the amino acid sequence of SEQ ID NO: 31, and an LC-CDR3 comprising the amino acid sequence of SEQ ID NO: 37.

In some embodiments, the anti-FcRn antibody comprises a V_{H} comprising the amino acid sequences of SEQ ID NOs: 4, 9 and 18, or a variant thereof comprising up to about 5 amino acid substitutions; and a V_{L} comprising the amino acid sequences of SEQ ID NOs: 26, 31 and 37, or a variant thereof comprising up to about 5 amino acid substitutions. In some embodiments, the anti-FcRn antibody comprises a V_{H} comprising the amino acid sequences of SEQ ID NOs: 4, 9 and 18; and a V_{L} comprising the amino acid sequences of SEQ ID NOs: 26, 31 and 37.

In some embodiments, the anti-FcRn antibody comprises a V_{H} comprising an HC-CDR1, an HC-CDR2 and an HC-CDR3 of the V_{H} comprising the amino acid sequence of SEQ ID NO: 44; and a V_{L} comprising an LC-CDR1, an LC-CDR2, and an LC-CDR3 of the V_{L} comprising the amino acid sequence of SEQ ID NO: 55.

In some embodiments, the anti-FcRn antibody comprises a V_{H} comprising one, two or three HC-CDRs of SEQ ID NO: 44.

In some embodiments, the anti-FcRn antibody comprises a V_{L} comprising one, two or three LC-CDRs of SEQ ID NO: 55.

In some embodiments, the anti-FcRn antibody comprises a V_{H} comprising HC-CDR1, HC-CDR2 and HC-CDR3 of the V_{H} of SEQ ID NO: 44, and a V_{L} comprising LC-CDR1, LC-CDR2 and LC-CDR3 of the V_{L} of SEQ ID NO: 55.

In some embodiments, the anti-FcRn antibody comprises a V_{H} comprising the amino acid sequence of SEQ ID NO: 44, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 55, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 44 and a V_{L} comprising the amino acid sequence of SEQ ID NO: 55.

In some embodiments, the anti-FcRn antibody comprises a V_{H} comprising: an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising an LC-CDR1 comprising the amino acid sequence of SEQ ID NO: 25, an LC-CDR2 comprising the amino acid sequence of SEQ ID NO: 32, and an LC-CDR3 comprising the amino acid sequence of SEQ ID NO: 38, or a variant thereof comprising up to about 5 amino acid substitutions in the LC-CDRs.

In some embodiments, the anti-FcRn antibody comprises a V_{H} comprising: an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 19; and a V_{L} comprising an LC-CDR1 comprising the amino acid sequence of SEQ ID NO: 25, an LC-CDR2 comprising the amino acid sequence of SEQ ID NO: 32, and an LC-CDR3 comprising the amino acid sequence of SEQ ID NO: 38.

In some embodiments, the anti-FcRn antibody comprises a V_{H} comprising the amino acid sequences of SEQ ID NOs: 1, 10 and 19, or a variant thereof comprising up to about 5 amino acid substitutions; and a V_{L} comprising the amino acid sequences of SEQ ID NOs: 25, 32 and 38, or a variant thereof comprising up to about 5 amino acid substitutions. In some embodiments, the anti-FcRn antibody comprises a V_{H} comprising the amino acid sequences of SEQ ID NOs: 1, 10 and 19; and a V_{L} comprising the amino acid sequences of SEQ ID NOs: 25, 32 and 38.

In some embodiments, the anti-FcRn antibody comprises a V_{H} comprising an HC-CDR1, an HC-CDR2 and an HC-CDR3 of the V_{H} comprising the amino acid sequence of SEQ ID NO: 45; and a V_{L} comprising an LC-CDR1, an LC-CDR2, and an LC-CDR3 of the V_{L} comprising the amino acid sequence of SEQ ID NO: 56.

In some embodiments, the anti-FcRn antibody comprises a V_{H} comprising one, two or three HC-CDRs of SEQ ID NO: 45.

In some embodiments, the anti-FcRn antibody comprises a V_{L} comprising one, two or three LC-CDRs of SEQ ID NO: 56.

In some embodiments, the anti-FcRn antibody comprises a V_{H} comprising HC-CDR1, HC-CDR2 and HC-CDR3 of the V_{H} of SEQ ID NO: 45, and a V_{L} comprising LC-CDR1, LC-CDR2 and LC-CDR3 of the V_{L} of SEQ ID NO: 56.

In some embodiments, the anti-FcRn antibody comprises a V_{H} comprising the amino acid sequence of SEQ ID NO: 45, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 56, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 45 and a V_{L} comprising the amino acid sequence of SEQ ID NO: 56.

In some embodiments, the anti-FcRn antibody comprises a V_{H} comprising: an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 38, or a variant thereof comprising up to about 5 amino acid substitutions in the LC-CDRs.

In some embodiments, the anti-FcRn antibody comprises a V_{H} comprising: an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 20; and a V_{L} comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 38.

In some embodiments, the anti-FcRn antibody comprises a V_{H} comprising the amino acid sequences of SEQ ID NOs: 1, 11 and 20, or a variant thereof comprising up to about 5 amino acid substitutions; and a V_{L} comprising the amino acid sequences of SEQ ID NOs: 23, 29 and 38, or a variant thereof comprising up to about 5 amino acid substitutions. In some embodiments, the anti-FcRn antibody comprises a V_{H} comprising the amino acid sequences of SEQ ID NOs: 1, 11 and 20; and a V_{L} comprising the amino acid sequences of SEQ ID NOs: 23, 29 and 38.

In some embodiments, the anti-FcRn antibody comprises a V_{H} comprising an HC-CDR1, an HC-CDR2 and an HC-CDR3 of the V_{H} comprising the amino acid sequence of SEQ ID NO: 46; and a V_{L} comprising an LC-CDR1, an LC-CDR2, and an LC-CDR3 of the V_{L} comprising the amino acid sequence of SEQ ID NO: 57.

In some embodiments, the anti-FcRn antibody comprises a V_{H} comprising one, two or three HC-CDRs of SEQ ID NO: 46.

In some embodiments, the anti-FcRn antibody comprises a V_{L} comprising one, two or three LC-CDRs of SEQ ID NO: 57.

In some embodiments, the anti-FcRn antibody comprises a V_{H} comprising HC-CDR1, HC-CDR2 and HC-CDR3 of the V_{H} of SEQ ID NO: 46, and a V_{L} comprising LC-CDR1, LC-CDR2 and LC-CDR3 of the V_{L} of SEQ ID NO: 57.

In some embodiments, the anti-FcRn antibody comprises a V_{H} comprising the amino acid sequence of SEQ ID NO: 46, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 57, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 46 and a V_{L} comprising the amino acid sequence of SEQ ID NO: 57.

In some embodiments, the anti-FcRn antibody comprises a V_{H} comprising: an HC-CDR1 comprising the amino acid sequence of SEQ ID NO: 5, an HC-CDR2 comprising the amino acid sequence of SEQ ID NO: 12, and an HC-CDR3 comprising the amino acid sequence of SEQ ID NO: 21, or a variant thereof comprising up to about 5 amino acid substitutions in the HC-CDRs; and a V_{L} comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39, or a variant thereof comprising up to about 5 amino acid substitutions in the LC-CDRs.

In some embodiments, the anti-FcRn antibody comprises a V_{H} comprising: an HC-CDR1 comprising the amino acid sequence of SEQ ID NO: 5, an HC-CDR2 comprising the amino acid sequence of SEQ ID NO: 12, and an HC-CDR3 comprising the amino acid sequence of SEQ ID NO: 21; and a V_{L} comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39.

In some embodiments, the anti-FcRn antibody comprises a V_{H} comprising the amino acid sequences of SEQ ID NOs: 5, 12 and 21, or a variant thereof comprising up to about 5 amino acid substitutions; and a V_{L} comprising the amino acid sequences of SEQ ID NOs: 27, 33 and 39, or a variant thereof comprising up to about 5 amino acid substitutions. In some embodiments, the anti-FcRn antibody comprises a V_{H} comprising the amino acid sequences of SEQ ID NOs: 5, 12 and 21; and a V_{L} comprising the amino acid sequences of SEQ ID NOs: 27, 33 and 39.

In some embodiments, the anti-FcRn antibody comprises a V_{H} comprising an HC-CDR1, an HC-CDR2 and an HC-CDR3 of the V_{H} comprising the amino acid sequence of SEQ ID NO: 47; and a V_{L} comprising an LC-CDR1, an LC-CDR2, and an LC-CDR3 of the V_{L} comprising the amino acid sequence of SEQ ID NO: 58.

In some embodiments, the anti-FcRn antibody comprises a V_{H} comprising one, two or three HC-CDRs of SEQ ID NO: 47.

In some embodiments, the anti-FcRn antibody comprises a V_{L} comprising one, two or three LC-CDRs of SEQ ID NO: 58.

In some embodiments, the anti-FcRn antibody comprises a V_{H} comprising HC-CDR1, HC-CDR2 and HC-CDR3 of the V_{H} of SEQ ID NO: 47, and a V_{L} comprising LC-CDR1, LC-CDR2 and LC-CDR3 of the V_{L} of SEQ ID NO: 58.

In some embodiments, the anti-FcRn antibody comprises a V_{H} comprising the amino acid sequence of SEQ ID NO: 47, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 58, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 47 and a V_{L} comprising the amino acid sequence of SEQ ID NO: 58.

In some embodiments, the anti-FcRn antibody comprises a V_{H} comprising: an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 21, or a variant thereof comprising up to about 5 amino acid substitutions in the HC-CDRs; and a V_{L} comprising an LC-CDR1 comprising the amino acid sequence of SEQ ID NO: 28, an LC-CDR2 comprising the amino acid sequence of SEQ ID NO: 33, and an LC-CDR3 comprising the amino acid sequence of SEQ ID NO: 40, or a variant thereof comprising up to about 5 amino acid substitutions in the LC-CDRs.

In some embodiments, the anti-FcRn antibody comprises a V_{H} comprising: an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 21; and a V_{L} comprising an LC-CDR1 comprising the amino acid sequence of SEQ ID NO: 28, an LC-CDR2 comprising the amino acid sequence of SEQ ID NO: 33, and an LC-CDR3 comprising the amino acid sequence of SEQ ID NO: 40.

In some embodiments, the anti-FcRn antibody comprises a V_{H} comprising the amino acid sequences of SEQ ID NOs: 5, 13 and 21, or a variant thereof comprising up to about 5 amino acid substitutions; and a V_{L} comprising the amino acid sequences of SEQ ID NOs: 28, 33 and 40, or a variant thereof comprising up to about 5 amino acid substitutions. In some embodiments, the anti-FcRn antibody comprises a V_{H} comprising the amino acid sequences of SEQ ID NOs: 5, 13 and 21; and a V_{L} comprising the amino acid sequences of SEQ ID NOs: 28, 33 and 40.

In some embodiments, the anti-FcRn antibody comprises a V_{H} comprising an HC-CDR1, an HC-CDR2 and an HC-CDR3 of the V_{H} comprising the amino acid sequence of SEQ ID NO: 48; and a V_{L} comprising an LC-CDR1, an LC-CDR2, and an LC-CDR3 of the V_{L} comprising the amino acid sequence of SEQ ID NO: 59.

In some embodiments, the anti-FcRn antibody comprises a V_{H} comprising one, two or three HC-CDRs of SEQ ID NO: 48.

In some embodiments, the anti-FcRn antibody comprises a V_{L} comprising one, two or three LC-CDRs of SEQ ID NO: 59.

In some embodiments, the anti-FcRn antibody comprises a V_{H} comprising HC-CDR1, HC-CDR2 and HC-CDR3 of the V_{H} of SEQ ID NO: 48, and a V_{L} comprising LC-CDR1, LC-CDR2 and LC-CDR3 of the V_{L} of SEQ ID NO: 59.

In some embodiments, the anti-FcRn antibody comprises a V_{H} comprising the amino acid sequence of SEQ ID NO: 48, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 59, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 48 and a V_{L} comprising the amino acid sequence of SEQ ID NO: 59.

In some embodiments, the anti-FcRn antibody comprises a V_{H} comprising: an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 22, or a variant thereof comprising up to about 5 amino acid substitutions in the HC-CDRs; and a V_{L} comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39, or a variant thereof comprising up to about 5 amino acid substitutions in the LC-CDRs.

In some embodiments, the anti-FcRn antibody comprises a V_{H} comprising: an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 22; and a V_{L} comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39.

In some embodiments, the anti-FcRn antibody comprises a V_{H} comprising the amino acid sequences of SEQ ID NOs: 6, 14 and 22, or a variant thereof comprising up to about 5 amino acid substitutions; and a V_{L} comprising the amino acid sequences of SEQ ID NOs: 27, 33 and 39, or a variant thereof comprising up to about 5 amino acid substitutions. In some embodiments, the anti-FcRn antibody comprises a V_{H} comprising the amino acid sequences of SEQ ID NOs: 6, 14 and 22; and a V_{L} comprising the amino acid sequences of SEQ ID NOs: 27, 33 and 39.

In some embodiments, the anti-FcRn antibody comprises a V_{H} comprising an HC-CDR1, an HC-CDR2 and an HC-CDR3 of the V_{H} comprising the amino acid sequence of SEQ ID NO: 49; and a V_{L} comprising an LC-CDR1, an LC-CDR2, and an LC-CDR3 of the V_{L} comprising the amino acid sequence of SEQ ID NO: 58.

In some embodiments, the anti-FcRn antibody comprises a V_{H} comprising one, two or three HC-CDRs of SEQ ID NO: 49.

In some embodiments, the anti-FcRn antibody comprises a V_{L} comprising one, two or three LC-CDRs of SEQ ID NO: 58.

In some embodiments, the anti-FcRn antibody comprises a V_{H} comprising HC-CDR1, HC-CDR2 and HC-CDR3 of the V_{H} of SEQ ID NO: 49, and a V_{L} comprising LC-CDR1, LC-CDR2 and LC-CDR3 of the V_{L} of SEQ ID NO: 58.

In some embodiments, the anti-FcRn antibody comprises a V_{H} comprising the amino acid sequence of SEQ ID NO: 49, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 58, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 49 and a V_{L} comprising the amino acid sequence of SEQ ID NO: 58.

In some embodiments, the amino acid substitutions described above are limited to “exemplary substitutions” shown in Table 4 of this application. In some embodiments, the amino acid substitutions are limited to “preferred substitutions” shown in Table 4 of this application.

In some embodiments, the anti-FcRn antibody comprises a V_{H} comprising an HC-CDR1, an HC-CDR2 and an HC-CDR3 of the V_{H} comprising the amino acid sequence of any one of SEQ ID NOs: 50-51; and a V_{L} comprising an LC-CDR1, an LC-CDR2, and an LC-CDR3 of the V_{L} comprising the amino acid sequence of any one of SEQ ID NOs: 60-61.

In some embodiments, the anti-FcRn antibody comprises a V_{H} comprising one, two or three HC-CDRs of SEQ ID NO: 50. In some embodiments, the anti-FcRn antibody comprises a V_{H} comprising one, two or three HC-CDRs of SEQ ID NO: 51.

In some embodiments, the anti-FcRn antibody comprises a V_{L} comprising one, two or three LC-CDRs of SEQ ID NO: 60. In some embodiments, the anti-FcRn antibody comprises a V_{L} comprising one, two or three LC-CDRs of SEQ ID NO: 61.

In some embodiments, the anti-FcRn antibody comprises a V_{H} comprising HC-CDR1, HC-CDR2 and HC-CDR3 of the V_{H} of SEQ ID NO: 50, and a V_{L} comprising LC-CDR1, LC-CDR2 and LC-CDR3 of the V_{L} of SEQ ID NO: 60.

In some embodiments, the anti-FcRn antibody comprises a V_{H} comprising HC-CDR1, HC-CDR2 and HC-CDR3 of the V_{H} of SEQ ID NO: 51, and a V_{L} comprising LC-CDR1, LC-CDR2 and LC-CDR3 of the V_{L} of SEQ ID NO: 61.

In some embodiments, the anti-FcRn antibody comprises a V_{H} comprising HC-CDR1, HC-CDR2 and HC-CDR3 of the V_{H} of SEQ ID NO: 50, and a V_{L} comprising LC-CDR1, LC-CDR2 and LC-CDR3 of the V_{L} of SEQ ID NO: 61.

In some embodiments, the anti-FcRn antibody comprises a V_{H} comprising the amino acid sequence of any one of SEQ ID NOs: 50-51, or a variant thereof having at least about 80% (such as at least about any one of 80%, 85%, 90%, 95%, 96%, 97%, 98%, or 99%) sequence identity, and a V_{L} comprising the amino acid sequence of any one of SEQ ID NOs: 60-61, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of any one of SEQ ID NOs: 50-51, and a V_{L} comprising the amino acid sequence of any one of SEQ ID NOs: 60-61.

In some embodiments, the anti-FcRn antibody comprises a V_{H} comprising the amino acid sequence of SEQ ID NO: 50, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 60, or a variant thereof having at least about 80% sequence identity. In some embodiments, the anti-FcRn antibody comprises a V_{H} comprising the amino acid sequence of SEQ ID NO: 50 and a V_{L} comprising the amino acid sequence of SEQ ID NO: 60.

In some embodiments, the anti-FcRn antibody comprises a V_{H} comprising the amino acid sequence of SEQ ID NO: 51, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 61, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 51 and a V_{L} comprising the amino acid sequence of SEQ ID NO: 61.

In some embodiments, the anti-FcRn antibody comprises a V_{H} comprising the amino acid sequence of SEQ ID NO: 50, or a variant thereof having at least about 80% (such as at least about any one of 80%, 85%, 90%, 95%, 96%, 97%, 98%, or 99%) sequence identity, and a V_{L} comprising the amino acid sequence of SEQ ID NO: 61, or a variant thereof having at least about 80% (such as at least about any one of 80%, 85%, 90%, 95%, 96%, 97%, 98%, or 99%) sequence identity. In some embodiments, the anti-FcRn antibody comprises a V_{H} comprising the amino acid sequence of SEQ ID NO: 50 and a V_{L} comprising the amino acid sequence of SEQ ID NO: 61.

In some embodiments, functional epitopes can be mapped by combinatorial alanine scanning. In this process, a combinatorial alanine-scanning strategy can be used to identify amino acids in the FcRn protein that are necessary for interaction with FcRn antibodies. In some embodiments, the epitope is conformational and crystal structure of anti-FcRn antibodies bound to FcRn may be employed to identify the epitopes.

In some embodiments, the present application provides antibodies which compete with any one of the FcRn antibodies described herein for binding to FcRn. In some embodiments, the present application provides antibodies which compete with any one of the anti-FcRn antibodies provided herein for binding to an epitope on the FcRn. In some embodiments, an anti-FcRn antibody is provided that binds to the same epitope as an anti-FcRn antibody comprising a V_{H} comprising the amino acid sequence of any one of SEQ ID NOs: 41-51, and a V_{L} comprising the amino acid sequence of any one of SEQ ID NOs: 52-61. In some embodiments, an anti-FcRn antibody is provided that specifically binds to FcRn competitively with an anti-FcRn antibody comprising a V_{H} comprising the amino acid sequence of any one of SEQ ID NOs: 41-51 and a V_{L} comprising the amino acid sequence of any one of SEQ ID NOs: 52-61.

In some embodiments, competition assays may be used to identify a monoclonal antibody that competes with an anti-FcRn antibody described herein for binding to FcRn. Competition assays can be used to determine whether two antibodies bind the same epitope by recognizing identical or sterically overlapping epitopes or one antibody competitively inhibits binding of another antibody to the antigen. In certain embodiments, such a competing antibody binds to the same epitope that is bound by an antibody described herein. Exemplary competition assays include, but are not limited to, routine assays such as those provided in Harlow and Lane (1988) Antibodies: A Laboratory Manual ch.14 (Cold Spring Harbor Laboratory, Cold Spring Harbor, N.Y.). Detailed exemplary methods for mapping an epitope to which an antibody binds are provided in Morris (1996) “Epitope Mapping Protocols”, in Methods in Molecular Biology vol. 66 (Humana Press, Totowa, N.J.). In some embodiments, two antibodies are said to bind to the same epitope if each blocks binding of the other by 50% or more. In some embodiments, the antibody that competes with an anti-FcRn antibody described herein is a chimeric, humanized or human antibody.

Exemplary anti-FcRn antibody sequences are shown in Tables 2 and 3, wherein the CDR numbering is according to the EU index of Kabat. Those skilled in the art will recognize that many algorithms are known for prediction of CDR positions and for delimitation of antibody heavy chain and light chain variable regions. Anti-FcRn antibodies comprising CDRs, V_{H} and/or V_{L} sequences from antibodies described herein, but based on prediction algorithms other than those exemplified in the tables below, are within the scope of this invention.

TABLE 2A
Exemplary anti-FcRn antibody CDR sequences.

| Antibody | | | |
|---|---|---|---|
| Name | HC-CDR1 | HC-CDR2 | HC-CDR3 |
| ZLP193 | SGYSWH | YIHDSGGTNYNPSLKS | EENYRYFDV |
| | (SEQ ID NO: 1) | (SEQ ID NO: 7) | (SEQ ID NO: 15) |
| ZLP37 | SYAMQ | YINPGTGYTNYNQKFKD | GGGLRGAMDY |
| | (SEQ ID NO: 2) | (SEQ ID NO: 8) | (SEQ ID NO: 16) |
| ZLP17 | SYTMH | YINPSSGYTNYNQKFKD | SGLRRVLDY |
| | (SEQ ID NO: 3) | (SEQ ID NO: 9) | (SEQ ID NO: 17) |
| ZLP22 | SYAMH | YINPSSGYTNYNQKFKD | KGLRYAVDY |
| | (SEQ ID NO: 4) | (SEQ ID NO: 9) | (SEQ ID NO: 18) |
| ZLP198 | SGYSWH | YIHNSGSTNYNPSLKS | EGYGGYWYFDV |
| | (SEQ ID NO: 1) | (SEQ ID NO: 10) | (SEQ ID NO: 19) |
| ZLP57 | SGYSWH | YIHSSGSTNYNPSLKS | EVYGNYYWYFDV |
| | (SEQ ID NO: 1) | (SEQ ID NO: 11) | (SEQ ID NO: 20) |
| ZLP44 | SYNMY | YIDPYNGGTRYNQKFKG | DGYNGAMDY |
| | (SEQ ID NO: 5) | (SEQ ID NO: 12) | (SEQ ID NO: 21) |
| ZLP64 | SYNMY | YIDPYNGGTNYNQKFKG | DGYNGAMDY |
| | (SEQ ID NO: 5) | (SEQ ID NO: 13) | (SEQ ID NO: 21) |
| ZLP42 | DTYMY | RIDPANGITKFDPKFQD | TAVMATEAMDR |
| | (SEQ ID NO: 6) | (SEQ ID NO: 14) | (SEQ ID NO: 22) |
| Antibody | | | |
| Name | LC-CDR1 | LC-CDR2 | LC-CDR3 |
| ZLP193 | SASSSVSYMH | STSNLAS | QQRSSYPPT |
| | (SEQ ID NO: 23) | (SEQ ID NO: 29) | (SEQ ID NO: 34) |
| ZLP37 | SANSSVNYMY | STSNLAS | HQRSSYPWT |
| | (SEQ ID NO: 24) | (SEQ ID NO: 29) | (SEQ ID NO: 35) |
| ZLP17 | SASSSVSYMY | DTSNLAS | QQWSSYPRT |
| | (SEQ ID NO: 25) | (SEQ ID NO: 30) | (SEQ ID NO: 36) |
| ZLP22 | KSSQSLLNSGNQKNYLA | GASTRES | QQHYSTPYT |
| | (SEQ ID NO: 26) | (SEQ ID NO: 31) | (SEQ ID NO: 37) |
| ZLP198 | SASSSVSYMY | GTSNLAS | QQRSSYPYT |
| | (SEQ ID NO: 25) | (SEQ ID NO: 32) | (SEQ ID NO: 38) |
| ZLP57 | SASSSVSYMH | STSNLAS | QQRSSYPYT |
| | (SEQ ID NO: 23) | (SEQ ID NO: 29) | (SEQ ID NO: 38) |
| ZLP44 | RASESVDSYGNSFMH | RASNLES | QQSNEDPYT |
| | (SEQ ID NO: 27) | (SEQ ID NO: 33) | (SEQ ID NO: 39) |
| ZLP64 | RASESVDRYGNSFMH | RASNLES | QQSNEDPLT |
| | (SEQ ID NO: 28) | (SEQ ID NO: 33) | (SEQ ID NO: 40) |
| ZLP42 | RASESVDSYGNSFMH | RASNLES | QQSNEDPYT |
| | (SEQ ID NO: 27) | (SEQ ID NO: 33) | (SEQ ID NO: 39) |

TABLE 2B
Exemplary anti-FcRn antibody CDR sequences.

| Antibody | | | |
|---|---|---|---|
| Name | HC-CDR1 | HC-CDR2 | HC-CDR3 |
| ZLP1-3-2 | SGYSWH | YIHDSGGTNYNPSLKS | EENYRYFDV |
| | (SEQ ID NO: 1) | (SEQ ID NO: 7) | (SEQ ID NO: 15) |
| ZLP1-3-2M | SGYSWH | YIHDSGGTNYNPSLKS | EENYRYFDV |
| | (SEQ ID NO: 1) | (SEQ ID NO: 7) | (SEQ ID NO: 15) |
| ZLP1-3-2F | SGYSWH | YIHDSGGTNYNPSLKS | EENYRYFDV |
| | (SEQ ID NO: 1) | (SEQ ID NO: 7) | (SEQ ID NO: 15) |
| Antibody | | | |
| Name | LC-CDR1 | LC-CDR2 | LC-CDR3 |
| ZLP1-3-2 | SASSSVSYMH | STSNLAS | QQRSSYPPT |
| | (SEQ ID NO: 23) | (SEQ ID NO: 29) | (SEQ ID NO: 34) |
| ZLP1-3-2M | SASSSVSYMH | STSNLAS | QQRSSYPPT |
| | (SEQ ID NO: 23) | (SEQ ID NO: 29) | (SEQ ID NO: 34) |
| ZLP1-3-2F | SASSSVSYMH | STSNLAS | QQRSSYPPT |
| | (SEQ ID NO: 23) | (SEQ ID NO: 29) | (SEQ ID NO: 34) |

TABLE 3A
Exemplary sequences.

| SEQ ID | | |
|---|---|---|
| NO | Description | Sequence |
| 41 | ZLP193 V_{H} | EVQLQESGPDLVKPSQSLSLTCTVTGYSFTSGYSWHWIRQFPGN |
| | | RLEWMGYIHDSGGTNYNPSLKSRISITRDTSKNQFFLQLNSVTTE |
| | | DTATYYCAREENYRYFDVWGAGTTVTVSS |
| 42 | ZLP37 V_{H} | EVQLQQSGVELARPGASVKMSCKASGYTFSSYAMQWVKKRPG |
| | | QGLEWIGYINPGTGYTNYNQKFKDKATLTADKSSSTAYMHLSS |
| | | LTSEDSAVYFCARGGGLRGAMDYWGQGTSVTVSS |
| 43 | ZLP17 V_{H} | QIQLLQSGAELARPGASVKMSCKASGYTFTSYTMHWVKQRPG |
| | | QGLEWIGYINPSSGYTNYNQKFKDKATLTADKSSSTAYMQLSSL |
| | | TSEGSAVYYCARSGLRRVLDYWGQGTSVTVSS |
| 44 | ZLP22 V_{H} | EVKLVESGAELARPGASVKMSCKASGYTFTSYAMHWVKQRPG |
| | | QGLEWIGYINPSSGYTNYNQKFKDKATLTADKSSSTAYMQLSSL |
| | | TSEDSAVYYCARKGLRYAVDYWGQGTSVTVSS |
| 45 | ZLP198 V_{H} | EVKLMESGPDLVKPSQSLSLTCTVTGYSITSGYSWHWIRHFPGK |
| | | KLEWMGYIHNSGSTNYNPSLKSRISISRETSKNQFFLQLNSVTTE |
| | | DTATYYCAREGYGGYWYFDVWGAGTSVTVSS |
| 46 | ZLP57 V_{H} | QVQLKESGPDLVKPSQSLSLTCTVTGYSITSGYSWHWIRQFPGN |
| | | KLEWMTYIHSSGSTNYNPSLKSRISITRDTSKNQFFLQLKSVTTE |
| | | DTATYYCAREVYGNYYWYFDVWGAGTTVTVSS |
| 47 | ZLP44 V_{H} | EVQLQQSGPELVKPGASVKVSCKASGYIFTSYNMYWMKQSHG |
| | | KSLEWIGYIDPYNGGTRYNQKFKGKATLTVDKSSSTAYMHLNS |
| | | LTSEDSAVYYCAVDGYNGAMDYWGQGTSVTVSS |
| 48 | ZLP64 V_{H} | QIQLLQSGPELVKPGASVKVSCKASGYIFTSYNMYWMKQSHGK |
| | | SLEWIGYIDPYNGGTNYNQKFKGKATLTVDKSSSTAYMHLNSL |
| | | TSEDSAVYYCAVDGYNGAMDYWGQGTSVTVSS |
| 49 | ZLP42 V_{H} | QVQLQQPGAELVKPGASVKLSCTASGFNIKDTYMYWVKQRPE |
| | | QGLEWIGRIDPANGITKFDPKFQDKATVTADTFSNIAYLHLSSLT |
| | | SEDTAVYYCTTTAVMATEAMDRWGQGTSVTVSS |
| 52 | ZLP193 V_{L} | DIQMMQSPAIMSASPGEKVTITCSASSSVSYMHWFQQKPGTSPK |
| | | LWIYSTSNLASGVPARFSGSGSGTSYSLTISRMEAEDAATYYCQ |
| | | QRSSYPPTFGDGTRLEIK |
| 53 | ZLP37 V_{L} | DIVLTQSPALMSASPGEKVTMTCSANSSVNYMYWYQQKPGSSP |
| | | KLWIYSTSNLASGVPARFSGSGSGTSYSLTISSMEAEDAATYYC |
| | | HQRSSYPWTFGGGTKLEIK |
| 54 | ZLP17 V_{L} | ENVLTQSPAILSASPGEKVTMTCSASSSVSYMYWYQQKPGSSPR |
| | | LLIYDTSNLASGVPVRFSGSGSGTSYSLTISRMEAEDAATYYCQ |
| | | QWSSYPRTFGGGTKLEIK |
| 55 | ZLP22 V_{L} | DIVMTQSPSSLSVSAGEKVTMSCKSSQSLLNSGNQKNYLAWYQ |
| | | QKPGQPPKLLIYGASTRESGVPDRFTGSGSGTDYTLTISSVQAED |
| | | LALYYCQQHYSTPYTFGGGTKLEIK |
| 56 | ZLP198 V_{L} | DIVLTQSPAIMSASPGEKVTMTCSASSSVSYMYWYQQKPGSSPK |
| | | PWIYGTSNLASGVPVRFSGSGSGTSYSLTISRMEAEDAATYYCQ |
| | | QRSSYPYTFGGGTKLEIK |
| 57 | ZLP57 V_{L} | DIVMTQSPAIMSASLGEEITLTCSASSSVSYMHWYQQKSGTSPK |
| | | LLIYSTSNLASGVPARFSGSGSGTSYSLTISRMEAEDAATYYCQQ |
| | | RSSYPYTFGGGTKLEIK |
| 58 | ZLP44 V_{L} | QIVLTQSPASLAVSLGQRATISCRASESVDSYGNSFMHWYQQKP |
| | ZLP42 V_{L} | GQPPKLLIYRASNLESGIPARFSGSGSRTDFTLTINPVEADDVAT |
| | | YYCQQSNEDPYTFGGGTKLEIK |
| 59 | ZLP64 V_{L} | DIVLTQSPASLTVSLGQRATISCRASESVDRYGNSFMHWYQQKP |
| | | GQPPKLLIYRASNLESGVPARFSGSGSRTDFTLTINPVEADDVAS |
| | | YYCQQSNEDPLTFGAGTKLELK |

TABLE 3B
Exemplary sequences.

| SEQ ID | | |
|---|---|---|
| NO | Description | Sequence |
| 50 | ZLP1-3-2 V_{H} | QVQLQESGPGLVKPSETLSLTCAVSGYSFTSGYSWHWIRQPPGK |
| | ZLP1-3-2F V_{H} | GLEWIGYIHDSGGTNYNPSLKSRVTISRDTSKNQFSLKLSSVTAA |
| | | DTAVYYCAREENYRYFDVWGQGTLVTVSS |
| 51 | ZLP1-3-2M V_{H} | QVQLQESGPGLVKPSETLSLTCTVTGYSFTSGYSWHWIRQPPGK |
| | | GLEWIGYIHDSGGTNYNPSLKSRVTISRDTSKNQFSLKLSSVTAA |
| | | DTAVYYCAREENYRYFDVWGQGTLVTVSS |
| 60 | ZLP1-3-2 V_{L} | EIVLTQSPATLSLSPGERATLSCSASSSVSYMHWFQQKPGQSPRL |
| | | WIYSTSNLASGIPARFSGSGSGTDYTLTISSLEPEDFAVYYCQQR |
| | | SSYPPTFGQGTKVEIK |
| 61 | ZLP1-3-2M V_{L} | EIVLTQSPATLSLSPGERATLSCSASSSVSYMHWFQQKPGQSPRL |
| | ZLP1-3-2F V_{L} | LIYSTSNLASGIPARFSGSGSGTDFTLTISSLEPEDFAVYYCQQRS |
| | | SYPPTFGQGTKVEIK |
| 62 | IgGI heavy | ASTKGPSVFPLAPSSKSTSGGTAALGCLVKDYFPEPVTVSWNSG |
| | chain constant | ALTSGVHTFPAVLQSSGLYSLSSVVTVPSSSLGTQTYICNVNHKP |
| | region | SNTKVDKKVEPKSCDKTHTCPPCPAPELLGGPSVFLFPPKPKDTL |
| | | MISRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGNVF |
| | | SCSVMHEALHNHYTQKSLSLSPGK |
| 63 | IgG4 heavy | ASTKGPSVFPLAPCSRSTSESTAALGCLVKDYFPEPVTVSWNSG |
| | chain constant | ALTSGVHTFPAVLQSSGLYSLSSVVTVPSSSLGTKTYTCNVDHK |
| | region | PSNTKVDKRVESKYGPPCPSCPAPEFLGGPSVFLFPPKPKDTLMI |
| | | SRTPEVTCVVVDVSQEDPEVQFNWYVDGVEVHNAKTKPREEQ |
| | | FNSTYRVVSVLTVLHQDWLNGKEYKCKVSNKGLPSSIEKTISKA |
| | | KGQPREPQVYTLPPSQEEMTKNQVSLTCLVKGFYPSDIAVEWES |
| | | NGQPENNYKTTPPVLDSDGSFFLYSRLTVDKSRWQEGNVFSCS |
| | | VMHEALHNHYTQKSLSLSLGK |
| 64 | Light chain | RTVAAPSVFIFPPSDEQLKSGTASVVCLLNNFYPREAKVQWKVD |
| | constant | NALQSGNSQESVTEQDSKDSTYSLSSTLTLSKADYEKHKVYACE |
| | region(kappa) | VTHQGLSSPVTKSFNRGEC |
| 65 | Light chain | GQPKAAPSVTLFPPSSEELQANKATLVCLISDFYPGAVTVAWKA |
| | constant | DSSPVKAGVETTTPSKQSNNKYAASSYLSLTPEQWKSHRSYSCQ |
| | region(lambda) | VTHEGSTVEKTVAPTECS |

### Full-Length Anti-FcRn Antibody

The anti-FcRn antibody in some embodiments is a full-length anti-FcRn antibody. In some embodiments, the full-length anti-FcRn antibody is an IgA, IgD, IgE, IgG, or IgM. In some embodiments, the full-length anti-FcRn antibody comprises IgG constant domains, such as constant domains of any of IgG1, IgG2, IgG3, and IgG4 including variants thereof. In some embodiments, the full-length anti-FcRn antibody comprises a lambda light chain constant region. In some embodiments, the full-length anti-FcRn antibody comprises a kappa light chain constant region. In some embodiments, the full-length anti-FcRn antibody is a full-length human anti-FcRn antibody. In some embodiments, the full-length anti-FcRn antibody comprises an Fc sequence of a mouse immunoglobulin. In some embodiments, the full-length anti-FcRn antibody comprises an Fc sequence that has been altered or otherwise changed so that it has enhanced antibody dependent cellular cytotoxicity (ADCC) or complement dependent cytotoxicity (CDC) effector function.

Thus, for example, in some embodiments, there is provided a full-length anti-FcRn antibody comprising IgG1 constant domains, wherein the anti-FcRn antibody specifically binds to FcRn. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG2 constant domains, wherein the anti-FcRn antibody specifically binds to FcRn. In some embodiments, the IgG2 is human IgG2. In some embodiments, the light chain constant region comprises or consists of the amino acid sequence of SEQ ID NO: 64. In some embodiments, the light chain constant region comprises or consists of the amino acid sequence of SEQ ID NO: 65.

In some embodiments, there is provided a full-length anti-FcRn antibody comprising IgG3 constant domains, wherein the anti-FcRn antibody specifically binds to FcRn. In some embodiments, the IgG3 is human IgG3. In some embodiments, the light chain constant region comprises or consists of the amino acid sequence of SEQ ID NO: 64. In some embodiments, the light chain constant region comprises or consists of the amino acid sequence of SEQ ID NO: 65.

In some embodiments, there is provided a full-length anti-FcRn antibody comprising IgG4 constant domains, wherein the anti-FcRn antibody specifically binds to FcRn. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64.

In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a) a heavy chain variable domain comprising an HC-CDR1 comprising the amino acid sequence of any one of SEQ ID NOs: 1-6, or a variant thereof comprising up to about 3 (such as about any of 1, 2, or 3) amino acid substitutions, an HC-CDR2 comprising the amino acid sequence of any one of SEQ ID NOs: 7-14, or a variant thereof comprising up to about 3 (such as about any of 1, 2, or 3) amino acid substitutions, and an HC-CDR3 comprising the amino acid sequence of any one of SEQ ID NOs: 15-22, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 23-28, or a variant thereof comprising up to about 3 (such as about any of 1, 2, or 3) amino acid substitutions, an LC-CDR2 comprising the amino acid sequence of any one of SEQ ID NOs: 29-33, or a variant thereof comprising up to about 3 (such as about any of 1, 2, or 3) amino acid substitutions, and an LC-CDR3 comprising the amino acid sequence of any one of SEQ ID NOs: 34-40, or a variant thereof comprising up to about 3 (such as about any of 1, 2, or 3) amino acid substitutions. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG2 constant domains, wherein the anti-FcRn antibody comprises a) a heavy chain variable domain comprising an HC-CDR1 comprising the amino acid sequence of any one of SEQ ID NOs: 1-6, or a variant thereof comprising up to about 3 (such as about any of 1, 2, or 3) amino acid substitutions, an HC-CDR2 comprising the amino acid sequence of any one of SEQ ID NOs: 7-14, or a variant thereof comprising up to about 3 (such as about any of 1, 2, or 3) amino acid substitutions, and an HC-CDR3 comprising the amino acid sequence of any one of SEQ ID NOs: 15-22, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 23-28, or a variant thereof comprising up to about 3 (such as about any of 1, 2, or 3) amino acid substitutions, an LC-CDR2 comprising the amino acid sequence of any one of SEQ ID NOs: 29-33, or a variant thereof comprising up to about 3 (such as about any of 1, 2, or 3) amino acid substitutions, and an LC-CDR3 comprising the amino acid sequence of any one of SEQ ID NOs: 34-40, or a variant thereof comprising up to about 3 (such as about any of 1, 2, or 3) amino acid substitutions. In some embodiments, the IgG2 is human IgG2. In some embodiments, the light chain constant region comprises or consists of the amino acid sequence of SEQ ID NO: 64. In some embodiments, the light chain constant region comprises or consists of the amino acid sequence of SEQ ID NO: 65.

In some embodiments, there is provided a full-length anti-FcRn antibody comprising IgG3 constant domains, wherein the anti-FcRn antibody comprises a) a heavy chain variable domain comprising an HC-CDR1 comprising the amino acid sequence of any one of SEQ ID NOs: 1-6, or a variant thereof comprising up to about 3 (such as about any of 1, 2, or 3) amino acid substitutions, an HC-CDR2 comprising the amino acid sequence of any one of SEQ ID NOs: 7-14, or a variant thereof comprising up to about 3 (such as about any of 1, 2, or 3) amino acid substitutions, and an HC-CDR3 comprising the amino acid sequence of any one of SEQ ID NOs: 15-22, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 23-28, or a variant thereof comprising up to about 3 (such as about any of 1, 2, or 3) amino acid substitutions, an LC-CDR2 comprising the amino acid sequence of any one of SEQ ID NOs: 29-33, or a variant thereof comprising up to about 3 (such as about any of 1, 2, or 3) amino acid substitutions, and an LC-CDR3 comprising the amino acid sequence of any one of SEQ ID NOs: 34-40, or a variant thereof comprising up to about 3 (such as about any of 1, 2, or 3) amino acid substitutions. In some embodiments, the IgG3 is human IgG3. In some embodiments, the light chain constant region comprises or consists of the amino acid sequence of SEQ ID NO: 64. In some embodiments, the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a) a heavy chain variable domain comprising an HC-CDR1 comprising the amino acid sequence of any one of SEQ ID NOs: 1-6, or a variant thereof comprising up to about 3 (such as about any of 1, 2, or 3) amino acid substitutions, an HC-CDR2 comprising the amino acid sequence of any one of SEQ ID NOs: 7-14, or a variant thereof comprising up to about 3 (such as about any of 1, 2, or 3) amino acid substitutions, and an HC-CDR3 comprising the amino acid sequence of any one of SEQ ID NOs: 15-22, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 23-28, or a variant thereof comprising up to about 3 (such as about any of 1, 2, or 3) amino acid substitutions, an LC-CDR2 comprising the amino acid sequence of any one of SEQ ID NOs: 29-33, or a variant thereof comprising up to about 3 (such as about any of 1, 2, or 3) amino acid substitutions, and an LC-CDR3 comprising the amino acid sequence of any one of SEQ ID NOs: 34-40, or a variant thereof comprising up to about 3 (such as about any of 1, 2, or 3) amino acid substitutions. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, the amino acid substitutions described above are limited to “exemplary substitutions” shown in Table 4 of this application. In some embodiments, the amino acid substitutions are limited to “preferred substitutions” shown in Table 4 of this application.

In some embodiments, there is provided a full-length anti-FcRn antibody comprising IgG1 constant domains, wherein the anti-FcRn antibody comprises a) a heavy chain variable domain comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 15; and b) a light chain variable domain comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 34. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a) a heavy chain variable domain comprising an HC-CDR1 comprising the amino acid sequence of SEQ ID NO: 2, an HC-CDR2 comprising the amino acid sequence of SEQ ID NO: 8, and an HC-CDR3 comprising the amino acid sequence of SEQ ID NO: 16; and b) a light chain variable domain comprising an LC-CDR1 comprising the amino acid sequence of SEQ ID NO: 24, an LC-CDR2 comprising the amino acid sequence of SEQ ID NO: 29, and an LC-CDR3 comprising the amino acid sequence of SEQ ID NO: 35. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a) a heavy chain variable domain comprising an HC-CDR1 comprising the amino acid sequence of SEQ ID NO: 3, an HC-CDR2 comprising the amino acid sequence of SEQ ID NO: 9, and an HC-CDR3 comprising the amino acid sequence of SEQ ID NO: 17; and b) a light chain variable domain comprising an LC-CDR1 comprising the amino acid sequence of SEQ ID NO: 25, an LC-CDR2 comprising the amino acid sequence of SEQ ID NO: 30, and an LC-CDR3 comprising the amino acid sequence of SEQ ID NO: 36. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a) a heavy chain variable domain comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 18; and b) a light chain variable domain comprising an LC-CDR1 comprising the amino acid sequence of SEQ ID NO: 26, an LC-CDR2 comprising the amino acid sequence of SEQ ID NO: 31, and an LC-CDR3 comprising the amino acid sequence of SEQ ID NO: 37. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a) a heavy chain variable domain comprising an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 25, an LC-CDR2 comprising the amino acid sequence of SEQ ID NO: 32, and an LC-CDR3 comprising the amino acid sequence of SEQ ID NO: 38. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a) a heavy chain variable domain comprising an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 20; and b) a light chain variable domain comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 38. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a) a heavy chain variable domain comprising an HC-CDR1 comprising the amino acid sequence of SEQ ID NO: 5, an HC-CDR2 comprising the amino acid sequence of SEQ ID NO: 12, and an HC-CDR3 comprising the amino acid sequence of SEQ ID NO: 21; and b) a light chain variable domain comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a) a heavy chain variable domain comprising an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 21; and b) a light chain variable domain comprising an LC-CDR1 comprising the amino acid sequence of SEQ ID NO: 28, an LC-CDR2 comprising the amino acid sequence of SEQ ID NO: 33, and an LC-CDR3 comprising the amino acid sequence of SEQ ID NO: 40. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a) a heavy chain variable domain comprising an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 22; and b) a light chain variable domain comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a) a heavy chain variable domain comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 15; and b) a light chain variable domain comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 34. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a) a heavy chain variable domain comprising an HC-CDR1 comprising the amino acid sequence of SEQ ID NO: 2, an HC-CDR2 comprising the amino acid sequence of SEQ ID NO: 8, and an HC-CDR3 comprising the amino acid sequence of SEQ ID NO: 16; and b) a light chain variable domain comprising an LC-CDR1 comprising the amino acid sequence of SEQ ID NO: 24, an LC-CDR2 comprising the amino acid sequence of SEQ ID NO: 29, and an LC-CDR3 comprising the amino acid sequence of SEQ ID NO: 35. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a) a heavy chain variable domain comprising an HC-CDR1 comprising the amino acid sequence of SEQ ID NO: 3, an HC-CDR2 comprising the amino acid sequence of SEQ ID NO: 9, and an HC-CDR3 comprising the amino acid sequence of SEQ ID NO: 17; and b) a light chain variable domain comprising an LC-CDR1 comprising the amino acid sequence of SEQ ID NO: 25, an LC-CDR2 comprising the amino acid sequence of SEQ ID NO: 30, and an LC-CDR3 comprising the amino acid sequence of SEQ ID NO: 36. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a) a heavy chain variable domain comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 18; and b) a light chain variable domain comprising an LC-CDR1 comprising the amino acid sequence of SEQ ID NO: 26, an LC-CDR2 comprising the amino acid sequence of SEQ ID NO: 31, and an LC-CDR3 comprising the amino acid sequence of SEQ ID NO: 37. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a) a heavy chain variable domain comprising an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 25, an LC-CDR2 comprising the amino acid sequence of SEQ ID NO: 32, and an LC-CDR3 comprising the amino acid sequence of SEQ ID NO: 38. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a) a heavy chain variable domain comprising an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 20; and b) a light chain variable domain comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 38. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a) a heavy chain variable domain comprising an HC-CDR1 comprising the amino acid sequence of SEQ ID NO: 5, an HC-CDR2 comprising the amino acid sequence of SEQ ID NO: 12, and an HC-CDR3 comprising the amino acid sequence of SEQ ID NO: 21; and b) a light chain variable domain comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a) a heavy chain variable domain comprising an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 21; and b) a light chain variable domain comprising an LC-CDR1 comprising the amino acid sequence of SEQ ID NO: 28, an LC-CDR2 comprising the amino acid sequence of SEQ ID NO: 33, and an LC-CDR3 comprising the amino acid sequence of SEQ ID NO: 40. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a) a heavy chain variable domain comprising an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 22; and b) a light chain variable domain comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of any one of SEQ ID NOs: 41-51, or a variant thereof having at least about 80% (such as at least about any one of 80%, 85%, 90%, 95%, 96%, 97%, 98%, or 99%) sequence identity, and a light chain variable domain comprising the amino acid sequence of any one of SEQ ID NOs: 52-61, or a variant thereof having at least about 80% (such as at least about any one of 80%, 85%, 90%, 95%, 96%, 97%, 98%, or 99%) sequence identity. In some embodiments, the IgG1 is human IgG1. In some embodiments, the heavy chain constant region comprises or consists of the amino acid sequence of SEQ ID NO: 62. In some embodiments, the light chain constant region comprises or consists of the amino acid sequence of SEQ ID NO: 64.

In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG2 constant domains, wherein the anti-FcRn antibody comprises a heavy chain variable domain comprising the amino acid sequence of any one of SEQ ID NOs: 41-51, or a variant thereof having at least about 80% (such as at least about any one of 80%, 85%, 90%, 95%, 96%, 97%, 98%, or 99%) sequence identity, and a light chain variable domain comprising the amino acid sequence of any one of SEQ ID NOs: 52-61, or a variant thereof having at least about 80% (such as at least about any one of 80%, 85%, 90%, 95%, 96%, 97%, 98%, or 99%) sequence identity. In some embodiments, the IgG2 is human IgG2. In some embodiments, the light chain constant region comprises or consists of the amino acid sequence of SEQ ID NO: 64. In some embodiments, the light chain constant region comprises or consists of the amino acid sequence of SEQ ID NO: 65.

In some embodiments, there is provided a full-length anti-FcRn antibody comprising IgG3 constant domains, wherein the anti-FcRn antibody comprises a heavy chain variable domain comprising the amino acid sequence of any one of SEQ ID NOs: 41-51, or a variant thereof having at least about 80% (such as at least about any one of 80%, 85%, 90%, 95%, 96%, 97%, 98%, or 99%) sequence identity, and a light chain variable domain comprising the amino acid sequence of any one of SEQ ID NOs: 52-61, or a variant thereof having at least about 80% (such as at least about any one of 80%, 85%, 90%, 95%, 96%, 97%, 98%, or 99%) sequence identity. In some embodiments, the IgG3 is human IgG3. In some embodiments, the light chain constant region comprises or consists of the amino acid sequence of SEQ ID NO: 64. In some embodiments, the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of any one of SEQ ID NOs: 41-51, or a variant thereof having at least about 80% (such as at least about any one of 80%, 85%, 90%, 95%, 96%, 97%, 98%, or 99%) sequence identity, and a light chain variable domain comprising the amino acid sequence of any one of SEQ ID NOs: 52-61, or a variant thereof having at least about 80% (such as at least about any one of 80%, 85%, 90%, 95%, 96%, 97%, 98%, or 99%) sequence identity. In some embodiments, the IgG4 is human IgG4. In some embodiments, the heavy chain constant region comprises or consists of the amino acid sequence of SEQ ID NO: 63. In some embodiments, the light chain constant region comprises or consists of the amino acid sequence of SEQ ID NO: 64.

In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 41 and a light chain variable domain comprising the amino acid sequence of SEQ ID NO: 52. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 42 and a light chain variable domain comprising the amino acid sequence of SEQ ID NO: 53. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 43 and a light chain variable domain comprising the amino acid sequence of SEQ ID NO: 54. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 44 and a light chain variable domain comprising the amino acid sequence of SEQ ID NO: 55. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 45 and a light chain variable domain comprising the amino acid sequence of SEQ ID NO: 56. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 46 and a light chain variable domain comprising the amino acid sequence of SEQ ID NO: 57. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 47 and a light chain variable domain comprising the amino acid sequence of SEQ ID NO: 58. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 48 and a light chain variable domain comprising the amino acid sequence of SEQ ID NO: 59. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 49 and a light chain variable domain comprising the amino acid sequence of SEQ ID NO: 58. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 50 and a light chain variable domain comprising the amino acid sequence of SEQ ID NO: 60. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 51 and a light chain variable domain comprising the amino acid sequence of SEQ ID NO: 61. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG1 constant domains, wherein the anti-FcRn antibody comprises a heavy chain variable domain comprising the amino acid sequence of SEQ ID NO: 50 and a light chain variable domain comprising the amino acid sequence of SEQ ID NO: 61. In some embodiments, the heavy chain constant region comprises or consists of the amino acid sequence of SEQ ID NO: 62. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 62 and the light chain constant region comprises or consists of the amino acid sequence of SEQ ID NO: 64. In some embodiments, the heavy chain constant region comprises or consists of the amino acid sequence of SEQ ID NO: 62 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 41 and a light chain variable domain comprising the amino acid sequence of SEQ ID NO: 52. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 42 and a light chain variable domain comprising the amino acid sequence of SEQ ID NO: 53. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 43 and a light chain variable domain comprising the amino acid sequence of SEQ ID NO: 54. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 44 and a light chain variable domain comprising the amino acid sequence of SEQ ID NO: 55. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 45 and a light chain variable domain comprising the amino acid sequence of SEQ ID NO: 56. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 46 and a light chain variable domain comprising the amino acid sequence of SEQ ID NO: 57. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 47 and a light chain variable domain comprising the amino acid sequence of SEQ ID NO: 58. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 48 and a light chain variable domain comprising the amino acid sequence of SEQ ID NO: 59. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 49 and a light chain variable domain comprising the amino acid sequence of SEQ ID NO: 58. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 50 and a light chain variable domain comprising the amino acid sequence of SEQ ID NO: 60. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 51 and a light chain variable domain comprising the amino acid sequence of SEQ ID NO: 61. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

In some embodiments, there is provided a full-length anti-FcRn antibody comprising IgG4 constant domains, wherein the anti-FcRn antibody comprises a heavy chain variable domain comprising the amino acid sequence of SEQ ID NO: 50 and a light chain variable domain comprising the amino acid sequence of SEQ ID NO: 61. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65. In some embodiments, the heavy chain constant region comprises or consists of the amino acid sequence of SEQ ID NO: 63 and the light chain constant region comprises or consists of the amino acid sequence of SEQ ID NO: 64. In some embodiments, the heavy chain constant region comprises or consists of the amino acid sequence of SEQ ID NO: 63 and the light chain constant region comprises or consists of the amino acid sequence of SEQ ID NO: 65.

### Binding Affinity

Binding affinity can be indicated by Kd, Koff, Kon, or Ka. The term “Koff”, as used herein, is intended to refer to the off-rate constant for dissociation of an antibody from the antibody/antigen complex, as determined from a kinetic selection set up. The term “Kon”, as used herein, is intended to refer to the on-rate constant for association of an antibody to the antigen to form the antibody/antigen complex. The term dissociation constant “Kd”, as used herein, refers to the dissociation constant of a particular antibody-antigen interaction, and describes the concentration of antigen required to occupy one half of all of the antibody-binding domains present in a solution of antibody molecules at equilibrium, and is equal to Koff/Kon. The measurement of Kd presupposes that all binding agents are in solution. In the case where the antibody is tethered to a cell wall, e.g., in a yeast expression system, the corresponding equilibrium rate constant is expressed as EC50, which gives a good approximation of Kd. The affinity constant, Ka, is the inverse of the dissociation constant, Kd.

The dissociation constant (Kd) is used as an indicator showing affinity of antibody moieties to antigens. For example, easy analysis is possible by the Scatchard method using antibodies marked with a variety of marker agents, as well as by using Biacore (made by Amersham Biosciences), analysis of biomolecular interactions by surface plasmon resonance, according to the user's manual and attached kit. The Kd value that can be derived using these methods is expressed in units of M. An antibody that specifically binds to a target may have a Kd of, for example, ≤10⁻⁷ M, ≤10⁻⁸ M, ≤10⁻⁹ M, ≤10⁻¹⁰ M, ≤10⁻¹¹ M, ≤10⁻¹² M, or ≤10⁻¹³ M.

Binding specificity of the antibody can be determined experimentally by methods known in the art. Such methods comprise, but are not limited to, Western blots, ELISA-, RIA-, ECL-, IRMA-, EIA-, BIAcore-tests and peptide scans.

In some embodiments, the anti-FcRn antibody specifically binds to a target FcRn with a Kd of about 10⁻⁷ M to about 10⁻¹³ M (such as about 10⁻⁷ M to about 10⁻¹³ M, about 10⁻⁸ M to about 10⁻¹³ M, about 10⁻⁹ M to about 10⁻¹³ M, or about 10⁻¹⁰ M to about 10⁻¹² M). Thus in some embodiments, the Kd of the binding between the anti-FcRn antibody and FcRn, is about 10⁻⁷ M to about 10⁻¹³ M, about 1×10⁻⁷ M to about 5×10⁻¹³ M, about 10⁻⁷ M to about 10⁻¹² M, about 10⁻⁷ M to about 10⁻¹¹ M, about 10⁻⁷ M to about 10⁻¹⁰ M, about 10⁻⁷ M to about 10⁻⁹ M, about 10⁻⁸ M to about 10⁻¹³ M, about 1×10⁻⁸ M to about 5×10⁻¹³ M, about 10⁻⁸ M to about 10⁻¹² M, about 10⁻⁸ M to about 10⁻¹¹ M, about 10⁻⁸ M to about 10⁻¹⁰ M, about 10⁻⁸ M to about 10⁻⁹ M, about 5×10⁻⁹ M to about 1×10⁻¹³ M, about 5×10⁻⁹ M to about 1×10⁻¹² M, about 5×10⁻⁹ M to about 1×10⁻¹¹ M, about 5×10⁻⁹ M to about 1×10⁻¹⁰ M, about 10⁻⁹ M to about 10⁻¹³ M, about 10⁻⁹ M to about 10⁻¹² M, about 10-9 M to about 10⁻¹¹ M, about 10⁻⁹ M to about 10⁻¹⁰ M, about 5×10⁻¹⁰ M to about 1×10⁻¹³ M, about 5×10⁻¹⁰ M to about 1×10⁻¹² M, about 5×10⁻¹⁰ M to about 1×10⁻¹¹ M, about 10⁻¹⁰ M to about 10⁻¹³ M, about 1×10⁻¹⁰ M to about 5×10⁻¹³ M, about 1×10⁻¹⁰ M to about 1×10⁻¹² M, about 1×10⁻¹⁰ M to about 5×10⁻¹² M, about 1×10⁻¹⁰ M to about 1×10⁻¹¹ M, about 10⁻¹¹ M to about 10⁻¹³ M, about 1×10⁻¹¹ M to about 5×10⁻¹³ M, about 10⁻¹¹ M to about 10⁻¹² M, or about 10⁻¹² M to about 10⁻¹³ M. In some embodiments, the Kd of the binding between the anti-FcRn antibody and FcRn is about 10-7 M to about 10⁻¹³ M.

In some embodiments, the Kd of the binding between the anti-FcRn antibody and a non-target is more than the Kd of the binding between the anti-FcRn antibody and the target, and is herein referred to in some embodiments as the binding affinity of the anti-FcRn antibody to the target (e.g., FcRn) is higher than that to a non-target. In some embodiments, the non-target is an antigen that is not FcRn. In some embodiments, the Kd of the binding between the anti-FcRn antibody (against FcRn) and a non-FcRn target can be at least about 10 times, such as about 10-100 times, about 100-1000 times, about 10³-10⁴ times, about 10⁴-10⁵ times, about 10⁵-10⁶ times, about 10⁶-10⁷ times, about 10⁷-10⁸ times, about 10⁸-10⁹ times, about 10⁹-10¹⁰ times, about 10¹⁰-10¹¹ times, or about 10¹¹-10¹² times.

In some embodiments, the anti-FcRn antibody binds to a non-target with a Kd of about 10⁻¹ M to about 10⁻⁶ M (such as about 10⁻¹ M to about 10⁻⁶ M, about 10⁻¹ M to about 10⁻⁵ M, or about 10⁻² M to about 10⁻⁴ M). In some embodiments, the non-target is an antigen that is not FcRn. Thus in some embodiments, the Kd of the binding between the anti-FcRn antibody and a non-FcRn target is about 10⁻¹ M to about 10⁻⁶ M, about 1×10⁻¹ M to about 5×10⁻⁶ M, about 10⁻¹ M to about 10⁻⁵ M, about 1×10⁻¹ M to about 5×10⁻⁵ M, about 10⁻¹ M to about 10⁻⁴ M, about 1×10⁻¹ M to about 5×10⁻⁴ M, about 10⁻¹ M to about 10⁻³ M, about 1×10⁻¹ M to about 5×10⁻³ M, about 10⁻¹ M to about 10⁻² M, about 10⁻² M to about 10⁻⁶ M, about 1×10⁻² M to about 5×10⁻⁶ M, about 10⁻² M to about 10⁻⁵ M, about 1×10⁻² M to about 5×10⁻⁵ M, about 10⁻² M to about 10⁻⁴ M, about 1×10⁻² M to about 5×10⁻⁴ M, about 10⁻² M to about 10⁻³ M, about 10⁻³ M to about 10⁻⁶ M, about 1×10⁻³ M to about 5×10⁻⁶ M, about 10⁻³ M to about 10⁻⁵ M, about 1×10⁻³ M to about 5×10⁻⁵ M, about 10⁻³ M to about 10⁻⁴ M, about 10⁻⁴ M to about 10⁻⁶ M, about 1×10⁻⁴ M to about 5×10⁻⁶ M, about 10⁻⁴ M to about 10⁻⁵ M, or about 10⁻⁵ M to about 10⁻⁶ M.

In some embodiments, when referring to that the anti-FcRn antibody specifically recognizes a target FcRn at a high binding affinity, and binds to a non-target at a low binding affinity, the anti-FcRn antibody will bind to the target FcRn with a Kd of about 10⁻⁷ M to about 10⁻¹³ M (such as about 10⁻⁷ M to about 10⁻¹³ M, about 10⁻⁸ M to about 10⁻¹³ M, about 10⁻⁹ M to about 10⁻¹³ M, or about 10⁻¹⁰ M to about 10⁻¹² M), and will bind to the non-target with a Kd of about 10⁻¹ M to about 10⁻⁶ M (such as about 10⁻¹ M to about 10⁻⁶ M, about 10⁻¹ M to about 10⁻⁵ M, or about 10⁻³ M to about 10⁻⁴ M).

### Nucleic Acids

Nucleic acid molecules encoding the anti-FcRn antibodies are also contemplated. In some embodiments, there is provided a nucleic acid (or a set of nucleic acids) encoding a full-length anti-FcRn antibody, including any of the full-length anti-FcRn antibodies described herein. In some embodiments, the nucleic acid (or a set of nucleic acids) encoding the anti-FcRn antibody described herein may further comprises a nucleic acid sequence encoding a peptide tag (such as protein purification tag, e.g., His-tag, HA tag).

Also contemplated here are isolated host cells comprising an anti-FcRn antibody, an isolated nucleic acid encoding the polypeptide components of the anti-FcRn antibody, or a vector comprising a nucleic acid encoding the polypeptide components of the anti-FcRn antibody described herein.

The present application also includes variants to these nucleic acid sequences. For example, the variants include nucleotide sequences that hybridize to the nucleic acid sequences encoding the anti-FcRn antibodies of the present application under at least moderately stringent hybridization conditions.

The present application also provides vectors in which a nucleic acid of the present application is inserted.

In brief summary, the expression of an anti-FcRn antibody (e.g., full-length anti-FcRn antibody) by a natural or synthetic nucleic acid encoding the anti-FcRn antibody can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acids of the present application may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the application provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Green and Sambrook (2013, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the application should not be limited to the use of constitutive promoters. Inducible promoters are also contemplated as part of the application. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the anti-FcRn antibody is inducible. In some embodiments, a nucleic acid sequence encoding the anti-FcRn antibody is operably linked to an inducible promoter, including any inducible promoter described herein.

### Inducible Promoters

The use of an inducible promoter provides a molecular switch capable of turning on expression of the polynucleotide sequence which it is operatively linked when such expression is desired, or turning off the expression when expression is not desired. Exemplary inducible promoter systems for use in eukaryotic cells include, but are not limited to, hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al. (1993) Science 262: 1019-1024) and ionizing radiation-regulated elements (e.g., see Manome, Y. et al. (1993) Biochemistry 32: 10607-10613; Datta, R. et al. (1992) Proc. Natl. Acad. Sci. USA 89: 1014-10153). Further exemplary inducible promoter systems for use in in vitro or in vivo mammalian systems are reviewed in Gingrich et al. (1998) Annual Rev. Neurosci 21:377-405. In some embodiments, the inducible promoter system for use to express the anti-FcRn antibody is the Tet system. In some embodiments, the inducible promoter system for use to express the anti-FcRn antibody is the lac repressor system from E. coli.

An exemplary inducible promoter system for use in the present application is the Tet system. Such systems are based on the Tet system described by Gossen et al. (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA's binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli (See Brown et al., Cell 49:603-612 (1987)). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided nucleic acid encoding a full-length anti-FcRn antibody according to any of the full-length anti-FcRn antibodies described herein. In some embodiments, the nucleic acid comprises one or more nucleic acid sequences encoding the heavy and light chains of the full-length anti-FcRn antibody. In some embodiments, each of the one or more nucleic acid sequences are contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Green and Sambrook (2013, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method of inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present application, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the application.

### Preparation of Anti-FcRn Antibodies

In some embodiments, the anti-FcRn antibody is a monoclonal antibody or derived from a monoclonal antibody. In some embodiments, the anti-FcRn antibody comprises V_{H} and V_{L} domains, or variants thereof, from the monoclonal antibody. In some embodiments, the anti-FcRn antibody further comprises C_{H}1 and C_{L} domains, or variants thereof, from the monoclonal antibody. Monoclonal antibodies can be prepared, e.g., using known methods in the art, including hybridoma methods, phage display methods, or using recombinant DNA methods. Additionally, exemplary phage display methods are described herein and in the Examples below.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be sub-cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub-clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, according to any of the anti-FcRn antibodies described herein, the anti-FcRn antibody comprises sequences from a clone selected from an antibody library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as scFv fragments or as Fab fragments.

Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The anti-FcRn antibodies can be prepared using phage display to screen libraries for anti-FcRn antibody moieties specific to the target FcRn. The library can be a human scFv phage display library having a diversity of at least 1×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully-synthetic phage display library. In some embodiments, the library is a non-human phage display library.

Phage clones that bind to the target FcRn with high affinity can be selected by iterative binding of phage to the target FcRn, which is bound to a solid support (such as, for example, beads for solution panning or mammalian cells for cell panning), followed by removal of non-bound phage and by elution of specifically bound phage. The bound phage clones are then eluted and used to infect an appropriate host cell, such as E. coli XL1-Blue, for expression and purification. The panning can be performed for multiple (such as about any of 2, 3, 4, 5, 6 or more) rounds with solution panning, cell panning, or a combination of both, to enrich for phage clones binding specifically to the target FcRn. Enriched phage clones can be tested for specific binding to the target FcRn by any methods known in the art, including for example ELISA and FACS.

An alternative method for screening antibody libraries is to display the protein on the surface of yeast cells. Wittrup et al. (U.S. Pat. Nos. 6,699,658 and 6,696,25 1) have developed a method for a yeast cell display library. In this yeast display system, a component involves the yeast agglutinin protein (Aga1), which is anchored to the yeast cell wall. Another component involves a second subunit of the agglutinin protein Aga2, which can display on the surface yeast cells through disulfide bonds to Aga1 protein. The protein Aga1 is expressed from a yeast chromosome after the Aga1 gene integration. A library of single chain variable fragments (scFv) is fused genetically to Aga2 sequence in the yeast display plasmid, which, after transformation, is maintained in yeast episomally with a nutritional marker. Both of the Aga1 and Aga2 proteins were expressed under the control of the galactose-inducible promoter.

Human antibody V gene repertoire (V_{H} and Vκ fragments) are obtained by PCR method using a pool of degenerate primers (Sblattero, D. & Bradbury, A. Immunotechnology 3, 271-278 1998). The PCR templates are from the commercially available RNAs or cDNAs, including PBMC, spleen, lymph nodes, bone marrow and tonsils. Separate V_{H} and Vκ PCR libraries were combined, then assembled together in the scFv format by overlap extension PCR (Sheets, M. D. et al., Proc. Natl. Acad. Sci. USA 95, 6157-6162 1998). To construct the yeast scFv display library, the resultant scFv PCR products are cloned into the yeast display plasmid in the yeasts by homologous recombination. (Chao, G, et al., Nat Protoc. 2006; 1(2):755-68. Miller K D, et al., Current Protocols in Cytometry 4.7.1-4.7.30, 2008).

The anti-FcRn antibodies can be discovered using mammalian cell display systems in which antibody moieties are displayed on the cell surface and those specific to the target FcRn are isolated by the antigen-guided screening method, as described in U.S. Pat. No. 7,732,195B2. A Chinese hamster ovary (CHO) cell library representing a large set of human IgG antibody genes can be established and used to discover the clones expressing high-affinity antibody genes. Another display system has been developed to enable simultaneous high-level cell surface display and secretion of the same protein through alternate splicing, where the displayed protein phenotype remains linked to genotype, allowing soluble secreted antibody to be simultaneously characterized in biophysical and cell-based functional assays. This approach overcomes many limitations of previous mammalian cell display, enabling direct selection and maturation of antibodies in the form of full-length, glycosylated IgGs (Peter M. Bowers, et al., Methods 2014,65:44-56). Transient expression systems are suitable for a single round of antigen selection before recovery of the antibody genes and therefore most useful for the selection of antibodies from smaller libraries. Stable episomal vectors offer an attractive alternative. Episomal vectors can be transfected at high efficiency and stably maintained at low copy number, permitting multiple rounds of panning and the resolution of more complex antibody libraries.

The IgG library is based on germline sequence V-gene segments joined to rearranged (D)J regions isolated from a panel of human donors. RNA collected from human blood samples was reverse-transcribed into cDNA, and the V_{H} and V_{κ} fragments were amplified using V_{H}- and V_{K}-specific primers and purified by gel extraction. IgG libraries were generated by sub-cloning the V_{H} and V_{K} fragments into the display vectors containing IgG1 or K constant regions respectively and then electroporating into or transducing 293T cells. To generate the scFv antibody display library, scFvs were generated by linking V_{H} and V_{K}, and then sub-cloned into the display vector, which were then electroporated into or transduce 293T cells. As we known, the IgG library is based on germline sequence V-gene segments joined to rearranged (D)J regions isolated from a panel of donors, the donor can be a mouse, rat, rabbit, or monkey.

Monoclonal antibodies can also be made by recombinant DNA methods, such as those described in U.S. Pat. No. 4,816,567. DNA encoding the monoclonal antibodies of the application can be readily isolated and sequenced using conventional procedures (e.g., by using oligonucleotide probes that are capable of binding specifically to genes encoding the heavy and light chains of murine antibodies). Hybridoma cells as described above or FcRn-specific phage clones of the application can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the application, or can be substituted for the variable domains of one antigen-combining site of an antibody of the application to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism.

### Human and Humanized Antibodies

The anti-FcRn antibodies (e.g., full-length anti-FcRn antibodies) can be humanized antibodies or human antibodies. Humanized forms of non-human (e.g., murine) antibody moieties are chimeric immunoglobulins, immunoglobulin chains, or fragments thereof (such as Fv, Fab, Fab′, F(ab′)₂, scFv, or other antigen-binding subsequences of antibodies) that typically contain minimal sequence derived from non-human immunoglobulin. Humanized antibody moieties include human immunoglobulins, immunoglobulin chains, or fragments thereof (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibody moieties can also comprise residues that are found neither in the recipient antibody nor in the imported CDR or framework sequences. In general, the humanized antibody can comprise substantially at least one, and typically two, variable domains, in which all or substantially all of the CDR regions correspond to those of a non-human immunoglobulin, and all or substantially all of the FR regions are those of a human immunoglobulin consensus sequence.

Generally, a humanized antibody has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibody moieties are antibody moieties (U.S. Pat. No. 4,816,567), wherein substantially less than an intact human variable domain has been substituted by the corresponding sequence from a non-human species. In practice, humanized antibody moieties are typically human antibody moieties in which some CDR residues and possibly some FR residues are substituted by residues from analogous sites in rodent antibodies.

As an alternative to humanization, human antibody moieties can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Anti-FcRn Antibody Variants

In some embodiments, amino acid sequences of the anti-FcRn antibody variants (e.g., full-length anti-FcRn antibody) provided herein are contemplated. For example, it may be desirable to improve the binding affinity and/or other biological properties of the antibody. Amino acid sequence of an antibody variant may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, anti-FcRn antibody variants having one or more amino acid substitutions are provided. Sites of interest for substitutional mutagenesis include the HVRs and FRs. Amino acid substitutions may be introduced into an antibody of interest and the products screened for a desired activity, e.g., improved bioactivity, retained/improved antigen binding, decreased immunogenicity, or improved ADCC or CDC.

Conservative substitutions are shown in Table 4 below.

TABLE 4
CONSERVATIVE SUBSTITUTIONS

| | | Preferred |
|---|---|---|
| Original | Exemplary | Sub- |
| Residue | Substitutions | stitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; | Tyr |
| | Tyr | |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped into different ciasses according to common side-chain properties:
a. hydrophobic: Norieucine, Met, Ala, Val, Leu, Ile;
b. neutral hydrophilic: Cys, Ser, Thr, Asn, Gin;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these ciasses for another class.

An exemplary substitutional variant is an affinity matured antibody, which may be conveniently generated, e.g., using phage display-based affinity maturation techniques. Briefly, one or more CDR residues are mutated and the variant antibody moieties displayed on phage and screened for a particular biological activity (e.g., bioactivity based on e.g., bioactivity based on IgG recycling assay or binding affinity). Alterations (e.g., substitutions) may be made in HVRs, e.g., to improve bioactivity based on IgG recycling assay or antibody affinity. Such alterations may be made in HVR “hotspots”, i.g., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant V_{H} and V_{L} being tested for binding affinity. Affinity maturation by constructing and reselecting from secondary libraries has been described, e.g., in Hoogenboom et al., in Methods in Molecular Biology 178:1-37 (O'Brien et al., ed., Human Press, Totowa, NJ, (2001)).

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant V_{H} and V_{L} sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., Ala or Glu) to determine whether the interaction of the antibody with antigen is affected. Further substitutions may be introduced at the amino acid locations to demonstrate functional sensitivity to the initial substitutions. Alternatively, or additionally, a crystal structure of an antigen-antibody complex can be determined to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Fe Region Variants

In some embodiments, one or more amino acid modifications may be introduced into the Fc region of an antibody (e.g., a full-length anti-FcRn antibody or anti-FcRn Fc fusion protein) provided herein, thereby generating an Fc region variant. In some embodiments, the Fc region variant has enhanced ADCC effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al., J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-FcRn antibody). The typical ADCC involves activation of NK cells by antibodies. An NK cell expresses CD16 which is an Fc receptor. This receptor recognizes, and binds to, the Fc portion of an antibody bound to the surface of a target cell. The most common Fc receptor on the surface of an NK cell is called CD16 or FcγRIII. Binding of the Fc receptor to the Fc region of an antibody results in NK cell activation, release of cytolytic granules and consequent target cell apoptosis. The contribution of ADCC to tumor cell killing can be measured with a specific test that uses NK-92 cells that have been transfected with a high-affinity FcR. Results are compared to wild-type NK-92 cells that do not express the FcR.

In some embodiments, the application contemplates an anti-FcRn antibody variant (such as a full-length anti-FcRn antibody variant) comprising an Fc region that possesses some but not all effector functions, which makes it a desirable candidate for applications in which the half-life of the anti-FcRn antibody in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al., Proc. Nat'l Acad. Sci. USA 83:7059-7063 (1986)) and Hellstrom, I. et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif; and CYTOTOX 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, the anti-FcRn antibody (such as a full-length anti-FcRn antibody) may include amino acid substitutions, additions, and/or deletions in the constant region (e.g., Fc region) of the antibody, e.g., resulting in reduced effector function, e.g., reduced complement-dependent cytolysis (CDC), antibody-dependent cell-mediated cytolysis (ADCC), and/or antibody-dependent cell-mediated phagocytosis (ADCP), and/or reduced B-cell killing. The constant regions are not directly involved in binding of an antibody to its target, but exhibit various effector functions, e.g., participation of the antibody in antibody-dependent cellular cytotoxicity. In some embodiments, the antibody is characterized by reduced binding (i.e., absence of binding) to human complement factor C1q and/or a human Fc receptor on Natural Killer (NK) cells. In other embodiments, the antibody is characterized by reduced binding (i.e., absence of binding) to human FcγRI, FcγRIIA, and/or FcγRIIIA. To alter or reduce antibody-dependent effector functions, such as CDC, ADCC, ADCP and/or B-cell killing, the antibody may be of the IgG class and contain one or more amino acid substitutions E233, L234, G236, D265, D270, N297, E318, K320, K322, A327, A330, P331 and/or P329 (numbering according to the European Kabat index (Sequences of Proteins of immunological Interest, 5th edition Public Health Service, National institute of Health, Bethesda, MID. (1991)).

In some embodiments, there is provided an anti-FcRn antibody (such as a full-length anti-FcRn antibody) variant comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fc region (EU numbering of residues). In some embodiments, the anti-FcRn antibody (e.g., full-length anti-FcRn antibody) variant comprises the following amino acid substitution in its variant Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, there is provided an anti-FcRn antibody (such as a full-length anti-FcRn antibody) variant comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Anti-FcRn antibodies (such as full-length anti-FcRn antibodies) comprising any of the Fc variants described herein, or combinations thereof, are contemplated.

### Glycosylation Variants

In some embodiments, an anti-FcRn antibody (such as a full-length anti-FcRn antibody) provided herein is altered to increase or decrease the extent to which the anti-FcRn antibody is glycosylated. Addition or deletion of glycosylation sites to an anti-FcRn antibody may be conveniently accomplished by altering the amino acid sequence of the anti-FcRn antibody or polypeptide portion thereof such that one or more glycosylation sites are created or removed.

Wherein the anti-FcRn antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-FcRn antibody of the application may be made in order to create anti-FcRn antibody variants with certain improved properties.

The N-glycans attached to the CH2 domain of Fc is heterogeneous. Antibodies or Fc fusion proteins generated in CHO cells are fucosylated by fucosyltransferase activity. See Shoji-Hosaka et al., J. Biochem. 2006, 140:777-83. Normally, a small percentage of naturally occurring afucosylated IgGs may be detected in human serum. N-glycosylation of the Fc is important for binding to FcγR; and afucosylation of the N-glycan increases Fc's binding capacity to FcγRIIIa. Increased FcγRIIIa binding can enhance ADCC, which can be advantageous in certain antibody therapeutic applications in which cytotoxicity is desirable.

In some embodiments, an enhanced effector function can be detrimental when Fc-mediated cytotoxicity is undesirable. In some embodiments, the Fc fragment or CH2 domain is not glycosylated. In some embodiments, the N-glycosylation site in the CH2 domain is mutated to prevent from glycosylation.

In some embodiments, anti-FcRn antibody (such as a full-length anti-FcRn antibody) variants are provided comprising an Fc region wherein a carbohydrate structure attached to the Fc region has reduced fucose or lacks fucose, which may improve ADCC function. Specifically, anti-FcRn antibodies are contemplated herein that have reduced fucose relative to the amount of fucose on the same anti-FcRn antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-FcRn antibody is one wherein less than about 50%, 40%, 30%, 20%, 10%, or 5% of the N-linked glycans thereon comprise fucose. For example, the amount of fucose in such an anti-FcRn antibody may be from 1% to 80%, from 1% to 65%, from 5% to 65% or from 20% to 40%. In some embodiments, the anti-FcRn antibody is one wherein none of the N-linked glycans thereon comprise fucose, i.e., wherein the anti-FcRn antibody is completely without fucose, or has no fucose or is afucosylated. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); US Pat Appl No US 2003/0157108 A1, Presta, L; and WO 2004/056312 A1, Adams et al., especially at Example 11), and knockout cell lines, such as α-1,6-fucosyltransferase gene, FUT8, knockout CHO cells (see, e.g., Yamane-Ohnuki et al., Biotech. Bioeng. 87: 614 (2004); Kanda, Y. et al., Biotechnol. Bioeng. 94(4):680-688 (2006); and WO2003/085107).

Anti-FcRn antibody (such as a full-length anti-FcRn antibody) variants are further provided with bisected oligosaccharides, e.g., in which a biantennary oligosaccharide attached to the Fc region of the anti-FcRn antibody is bisected by GlcNAc. Such anti-FcRn antibody (such as a full-length anti-FcRn antibody) variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Anti-FcRn antibody (such as full-length anti-FcRn antibody) variants with at least one galactose residue in the oligosaccharide attached to the Fc region are also provided. Such anti-FcRn antibody variants may have improved CDC function. Such antibody variants are described, e.g., in WO 1997/30087 (Patel et al.); WO 1998/58964 (Raju, S.); and WO 1999/22764 (Raju, S.).

In some embodiments, the anti-FcRn antibody (such as a full-length anti-FcRn antibody) variants comprising an Fc region are capable of binding to an FcγRIII. In some embodiments, the anti-FcRn antibody (such as a full-length anti-FcRn antibody) variants comprising an Fc region have ADCC activity in the presence of human effector cells (e.g., T cell) or have increased ADCC activity in the presence of human effector cells compared to the otherwise same anti-FcRn antibody (such as a full-length anti-FcRn antibody) comprising a human wild-type IgG1Fc region.

### Cysteine Engineered Variants

In some embodiments, it may be desirable to create cysteine engineered anti-FcRn antibodies (such as a full-length anti-FcRn antibody) in which one or more amino acid residues are substituted with cysteine residues. In some embodiments, the substituted residues occur at accessible sites of the anti-FcRn antibody. By substituting those residues with cysteine, reactive thiol groups are thereby positioned at accessible sites of the anti-FcRn antibody and may be used to conjugate the anti-FcRn antibody to other moieties, such as drug moieties or linker-drug moieties, to create an anti-FcRn immunoconjugate, as described further herein. Cysteine engineered anti-FcRn antibodies (e.g., full-length anti-FcRn antibodies) may be generated as described, e.g., in U.S. Pat. No. 7,521,541.

### Derivatives

In some embodiments, an anti-FcRn antibody (such as a full-length anti-FcRn antibody) provided herein may be further modified to contain additional non-proteinaceous moieties that are known in the art and readily available. The moieties suitable for derivatization of the anti-FcRn antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the anti-FcRn antibody may vary, and if more than one polymer are attached, they can be the same or different molecules. In general, the number and/or type of polymers used for derivatization can be determined based on considerations including, but not limited to, the particular properties or functions of anti-FcRn antibody to be improved, whether the anti-FcRn antibody derivative will be used in a therapy under defined conditions, etc.

### Pharmaceutical Compositions

Also provided herein are compositions (such as pharmaceutical compositions, also referred to herein as formulations) comprising any of the anti-FcRn antibodies (such as a full-length anti-FcRn antibody), nucleic acids encoding the antibodies, vectors comprising the nucleic acids encoding the antibodies, or host cells comprising the nucleic acids or vectors described herein. In some embodiments, there is provided a pharmaceutical composition comprising any one of the anti-FcRn antibodies described herein and a pharmaceutically acceptable carrier.

Suitable formulations of the anti-FcRn antibodies are obtained by mixing an anti-FcRn antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the anti-FcRn antibodies of this application into cells.

The formulation herein may also contain one or more active compounds in addition to the anti-FcRn antibody (such as a full-length anti-FcRn antibody) as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent in addition to the anti-FcRn antibody. Such molecules are suitably present in combination in amounts that are effective for the purpose intended. The effective amount of such other agents depends on the amount of anti-FcRn antibody present in the formulation, the type of disease or disorder or treatment, and other factors discussed above. These are generally used in the same dosages and with administration routes as described herein or about from 1 to 99% of the heretofore employed dosages.

The anti-FcRn antibodies (e.g., full-length anti-FcRn antibodies) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Sustained-release preparations may be prepared.

Sustained-release preparations of the anti-FcRn antibodies (e.g., full-length anti-FcRn antibodies) can be prepared. Suitable examples of sustained-release preparations include semipermeable matrices of solid hydrophobic polymers containing the antibody (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D (−)-3-hydroxybutyric acid. While polymers such as ethylene-vinyl acetate and lactic acid-glycolic acid enable release of molecules for over 100 days, certain hydro gels release proteins for shorter time periods. When encapsulated antibody remain in the body for a long time, they can denature or aggregate as a result of exposure to moisture at 37° C., resulting in a loss of biological activity and possible changes in immunogenicity. Rational strategies can be devised for stabilization of anti-FcRn antibodies depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the anti-FcRn antibody (such as a full-length anti-FcRn antibody) is formulated in a buffer comprising a citrate, NaCl, acetate, succinate, glycine, polysorbate 80 (Tween 80), or any combination of the foregoing.

The formulations to be used for in vivo administration must be sterile. This is readily accomplished by, e.g., filtration through sterile filtration membranes.

### Methods of Treatment Using Anti-FcRn Antibodies

The anti-FcRn antibodies (e.g., full-length anti-FcRn antibodies) and/or compositions of the application can be administered to individuals (e.g., mammals such as humans) to promote clearance of autoantibodies in a subject, to suppress antigen presentation in a subject, to block an immune response (e.g., block an immune complex-based activation of the immune response in a subject), to treat immunological diseases (e.g., autoimmune disorders) and inflammatory diseases in a subject. Autoimmune disorders refer to a class of diseases in which a subject's own antibodies react with host tissue or in which immune effector T cells are autoreactive to endogenous self-peptides and cause destruction of tissue. Thus, an immune response is mounted against a subject's own antigens, referred to as self-antigens. A “self-antigen” as used herein refers to an antigen of a normal host tissue. Normal host tissue does not include neoplastic cells. These diseases include, but are not limited to, Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome.

In some embodiments, the present application provides a method of modulating the interaction between FcRn and IgG Fc which comprises contacting FcRn in a cell or in a subject with an FcRn antibody or antigen-binding fragment, or an effective amount of a composition (such as a pharmaceutical composition) comprising any one of the anti-FcRn antibody (e.g., a full-length anti-FcRn antibody) described herein. In some embodiments, the modulation inhibits the interaction between FcRn and IgG Fc. Thus, provided is a method of promoting antibody degradation by a cell or in an individual. In some embodiments, the antibody is an autoantibody. In some embodiments, the individual is human.

In some embodiments, the present application provides a method of treating or ameliorating an IgG-mediated disease in an individual, which comprises administering to the subject an FcRn antibody or antigen binding fragment, or an effective amount of a composition (such as a pharmaceutical composition) comprising any one of the anti-FcRn antibody (e.g., a full-length anti-FcRn antibody) described herein in an amount effective to treat or ameliorate the IgG-mediated disease. Such IgG-mediated diseases may be those that involve pathogenic IgG antibodies in monomeric form or as IgG-containing immune complexes (IC) and include coagulopathies, vasculitides, collagen disorders, dermatological diseases, neurological diseases, inflammatory bowel diseases, and organ-specific disorders. In some embodiments, the individual is human.

In some embodiments, the present application provides a method of blocking the transmission of pathogenic antibodies across the placenta that comprises administering to a pregnant mammal in need thereof a therapeutically effective amount of an anti-FcRn antibody or antigen binding fragment, or a composition (such as a pharmaceutical composition) comprising any one of the anti-FcRn antibody (e.g., a full-length anti-FcRn antibody) described herein.

In some embodiments, the present application provides a method of inhibiting immune complex (IC) binding by FcRn, which comprises contacting FcRn in a cell or in an individual with an FcRn antibody or antigen-binding fragment, or a composition (such as a pharmaceutical composition) comprising any one of the anti-FcRn antibody (e.g., a full-length anti-FcRn antibody) described herein. Accordingly, also provided is a method of inhibiting presentation of an immune complexed antigen by an antigen presenting cell (APC), which comprises contacting the APC with an amount of an anti-FcRn antibody, antigen-binding fragment, or a composition (such as a pharmaceutical composition) comprising any one of the anti-FcRn antibody (e.g., a full-length anti-FcRn antibody) described herein. In some embodiments, the individual is human.

In some embodiments, the present application provides a method of increasing the clearance of ICs from an individual which comprises administering to an individual in need thereof an anti-FcRn antibody, antigen-binding fragment, or a composition (such as a pharmaceutical composition) comprising any one of the anti-FcRn antibody (e.g., a full-length anti-FcRn antibody) described herein. Such methods may be used to treat vasculitides that are IC-mediated. In some embodiments, the individual is human.

In some embodiments, the present application provides a method of inhibiting secretion of an inflammatory cytokine by an antigen presenting cell (APC), which comprises contacting the APC with an anti-FcRn antibody, antigen-binding fragment, or a composition (such as a pharmaceutical composition) comprising any one of the anti-FcRn antibody (e.g., a full-length anti-FcRn antibody) described herein. Non-limiting examples of inflammatory cytokines include, e.g., interleukin-12 (IL-12), interleukin-6 (IL-6) and interferon 7 (IFN 7).

In some embodiments, the present application provides a method of inhibiting T cell activation by an antigen presenting cell which comprises contacting the antigen presenting cell with an anti-FcRn antibody, antigen-binding fragment, or a composition (such as a pharmaceutical composition) comprising any one of the anti-FcRn antibody (e.g., a full-length anti-FcRn antibody) described herein.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 15, or a variant thereof comprising up to 5 amino acid substitutions; and a V_{L} comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 34, or a variant thereof comprising up to 5 amino acid substitutions. In some embodiments, the anti-FcRn antibody is a full-length antibody.

In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

In some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising the amino acid sequence SEQ ID NO: 41 or a variant thereof having at least about 80% (such as at least about any one of 80%, 85%, 90%, 95%, 96%, 97%, 98%, or 99%) sequence identity to the amino acid sequence of SEQ ID NO: 41, and a V_{L} comprising the amino acid sequence of SEQ ID NO: 52, or a variant thereof having at least about 80% (such as at least about any one of 80%, 85%, 90%, 95%, 96%, 97%, 98%, or 99%) sequence identity to the amino acid sequence of SEQ ID NO: 52.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising an HC-CDR1 comprising the amino acid sequence of SEQ ID NO: 2, an HC-CDR2 comprising the amino acid sequence of SEQ ID NO: 8, and an HC-CDR3 comprising the amino acid sequence of SEQ ID NO: 16, or a variant thereof comprising up to 5 amino acid substitutions; and a V_{L} comprising an LC-CDR1 comprising the amino acid sequence of SEQ ID NO: 24, an LC-CDR2 comprising the amino acid sequence of SEQ ID NO: 29, and an LC-CDR3 comprising the amino acid sequence of SEQ ID NO: 35, or a variant thereof comprising up to 5 amino acid substitutions. In some embodiments, the anti-FcRn antibody is a full-length antibody. In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

In some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising the amino acid sequence SEQ ID NO: 42 or a variant thereof having at least about 80% (such as at least about any one of 80%, 85%, 90%, 95%, 96%, 97%, 98%, or 99%) sequence identity to the amino acid sequence of SEQ ID NO: 42, and a V_{L} comprising the amino acid sequence of SEQ ID NO: 53, or a variant thereof having at least about 80% (such as at least about any one of 80%, 85%, 90%, 95%, 96%, 97%, 98%, or 99%) sequence identity to the amino acid sequence of SEQ ID NO: 53.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising an HC-CDR1 comprising the amino acid sequence of SEQ ID NO: 3, an HC-CDR2 comprising the amino acid sequence of SEQ ID NO: 9, and an HC-CDR3 comprising the amino acid sequence of SEQ ID NO: 17, or a variant thereof comprising up to 5 amino acid substitutions; and a V_{L} comprising an LC-CDR1 comprising the amino acid sequence of SEQ ID NO: 25, an LC-CDR2 comprising the amino acid sequence of SEQ ID NO: 30, and an LC-CDR3 comprising the amino acid sequence of SEQ ID NO: 36, or a variant thereof comprising up to 5 amino acid substitutions. In some embodiments, the anti-FcRn antibody is a full-length antibody.

In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

In some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising the amino acid sequence SEQ ID NO: 43 or a variant thereof having at least about 80% (such as at least about any one of 80%, 85%, 90%, 95%, 96%, 97%, 98%, or 99%) sequence identity to the amino acid sequence of SEQ ID NO: 43, and a V_{L} comprising the amino acid sequence of SEQ ID NO: 54, or a variant thereof having at least about 80% (such as at least about any one of 80%, 85%, 90%, 95%, 96%, 97%, 98%, or 99%) sequence identity to the amino acid sequence of SEQ ID NO: 54.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising an HC-CDR1 comprising the amino acid sequence of SEQ ID NO: 4, an HC-CDR2 comprising the amino acid sequence of SEQ ID NO: 9, and an HC-CDR3 comprising the amino acid sequence of SEQ ID NO: 18, or a variant thereof comprising up to 5 amino acid substitutions; and a V_{L} comprising an LC-CDR1 comprising the amino acid sequence of SEQ ID NO: 26, an LC-CDR2 comprising the amino acid sequence of SEQ ID NO: 31, and an LC-CDR3 comprising the amino acid sequence of SEQ ID NO: 37, or a variant thereof comprising up to 5 amino acid substitutions. In some embodiments, the anti-FcRn antibody is a full-length antibody. In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

In some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising the amino acid sequence SEQ ID NO: 44 or a variant thereof having at least about 80% (such as at least about any one of 80%, 85%, 90%, 95%, 96%, 97%, 98%, or 99%) sequence identity to the amino acid sequence of SEQ ID NO: 44, and a V_{L} comprising the amino acid sequence of SEQ ID NO: 55, or a variant thereof having at least about 80% (such as at least about any one of 80%, 85%, 90%, 95%, 96%, 97%, 98%, or 99%) sequence identity to the amino acid sequence of SEQ ID NO: 55.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 19, or a variant thereof comprising up to 5 amino acid substitutions; and a V_{L} comprising an LC-CDR1 comprising the amino acid sequence of SEQ ID NO: 25, an LC-CDR2 comprising the amino acid sequence of SEQ ID NO: 32, and an LC-CDR3 comprising the amino acid sequence of SEQ ID NO: 38, or a variant thereof comprising up to 5 amino acid substitutions. In some embodiments, the anti-FcRn antibody is a full-length antibody. In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

In some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising the amino acid sequence SEQ ID NO: 45 or a variant thereof having at least about 80% (such as at least about any one of 80%, 85%, 90%, 95%, 96%, 97%, 98%, or 99%) sequence identity to the amino acid sequence of SEQ ID NO: 45, and a V_{L} comprising the amino acid sequence of SEQ ID NO: 56, or a variant thereof having at least about 80% (such as at least about any one of 80%, 85%, 90%, 95%, 96%, 97%, 98%, or 99%) sequence identity to the amino acid sequence of SEQ ID NO: 56.

For example, in some embodiments, there is provided a method of treating an individual having a disease described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 20, or a variant thereof comprising up to 5 amino acid substitutions; and a V_{L} comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 38, or a variant thereof comprising up to 5 amino acid substitutions. In some embodiments, the anti-FcRn antibody is a full-length antibody. In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

In some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising the amino acid sequence SEQ ID NO: 46 or a variant thereof having at least about 80% (such as at least about any one of 80%, 85%, 90%, 95%, 96%, 97%, 98%, or 99%) sequence identity to the amino acid sequence of SEQ ID NO: 46, and a V_{L} comprising the amino acid sequence of SEQ ID NO: 57, or a variant thereof having at least about 80% (such as at least about any one of 80%, 85%, 90%, 95%, 96%, 97%, 98%, or 99%) sequence identity to the amino acid sequence of SEQ ID NO: 57.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising an HC-CDR1 comprising the amino acid sequence of SEQ ID NO: 5, an HC-CDR2 comprising the amino acid sequence of SEQ ID NO: 12, and an HC-CDR3 comprising the amino acid sequence of SEQ ID NO: 21, or a variant thereof comprising up to 5 amino acid substitutions; and a V_{L} comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39, or a variant thereof comprising up to 5 amino acid substitutions. In some embodiments, the anti-FcRn antibody is a full-length antibody. In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

In some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising the amino acid sequence SEQ ID NO: 47 or a variant thereof having at least about 80% (such as at least about any one of 80%, 85%, 90%, 95%, 96%, 97%, 98%, or 99%) sequence identity to the amino acid sequence of SEQ ID NO: 47, and a V_{L} comprising the amino acid sequence of SEQ ID NO: 58, or a variant thereof having at least about 80% (such as at least about any one of 80%, 85%, 90%, 95%, 96%, 97%, 98%, or 99%) sequence identity to the amino acid sequence of SEQ ID NO: 58.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 21, or a variant thereof comprising up to 5 amino acid substitutions; and a V_{L} comprising an LC-CDR1 comprising the amino acid sequence of SEQ ID NO: 28, an LC-CDR2 comprising the amino acid sequence of SEQ ID NO: 33, and an LC-CDR3 comprising the amino acid sequence of SEQ ID NO: 40, or a variant thereof comprising up to 5 amino acid substitutions. In some embodiments, the anti-FcRn antibody is a full-length antibody. In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

In some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising the amino acid sequence SEQ ID NO: 48 or a variant thereof having at least about 80% (such as at least about any one of 80%, 85%, 90%, 95%, 96%, 97%, 98%, or 99%) sequence identity to the amino acid sequence of SEQ ID NO: 48, and a V_{L} comprising the amino acid sequence of SEQ ID NO: 59, or a variant thereof having at least about 80% (such as at least about any one of 80%, 85%, 90%, 95%, 96%, 97%, 98%, or 99%) sequence identity to the amino acid sequence of SEQ ID NO: 59.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 22, or a variant thereof comprising up to 5 amino acid substitutions; and a V_{L} comprising an LC-CDR1 comprising the amino acid sequence of SEQ ID NO: 27, an LC-CDR2 comprising the amino acid sequence of SEQ ID NO: 33, and an LC-CDR3 comprising the amino acid sequence of SEQ ID NO: 39, or a variant thereof comprising up to 5 amino acid substitutions. In some embodiments, the anti-FcRn antibody is a full-length antibody. In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

In some embodiments, there is provided a method of treating an individual having a disease or condition described above (e.g., autoimmune disorders and inflammatory diseases) comprising administering to the individual an effective amount of a pharmaceutical composition comprising an anti-FcRn antibody (e.g., full-length anti-FcRn antibody) comprising a V_{H} comprising the amino acid sequence SEQ ID NO: 49 or a variant thereof having at least about 80% (such as at least about any one of 80%, 85%, 90%, 95%, 96%, 97%, 98%, or 99%) sequence identity to the amino acid sequence of SEQ ID NO: 49, and a V_{L} comprising the amino acid sequence of SEQ ID NO: 58, or a variant thereof having at least about 80% (such as at least about any one of 80%, 85%, 90%, 95%, 96%, 97%, 98%, or 99%) sequence identity to the amino acid sequence of SEQ ID NO: 58.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody comprises a V_{H} comprising the amino acid sequence of SEQ ID NO: 50 or a variant thereof having at least about 80% (such as at least about any one of 80%, 85%, 90%, 95%, 96%, 97%, 98%, or 99%) sequence identity to the amino acid sequence of SEQ ID NO: 50, and a V_{L} comprising the amino acid sequence of SEQ ID NO: 60, or a variant thereof having at least about 80% (such as at least about any one of 80%, 85%, 90%, 95%, 96%, 97%, 98%, or 99%) sequence identity to the amino acid sequence of SEQ ID NO: 60. In some embodiments, the anti-FcRn antibody is a full-length antibody. In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody comprises a V_{H} comprising the amino acid sequence of SEQ ID NO: 51 or a variant thereof having at least about 80% (such as at least about any one of 80%, 85%, 90%, 95%, 96%, 97%, 98%, or 99%) sequence identity to the amino acid sequence of SEQ ID NO: 51, and a V_{L} comprising the amino acid sequence of SEQ ID NO: 61, or a variant thereof having at least about 80% (such as at least about any one of 80%, 85%, 90%, 95%, 96%, 97%, 98%, or 99%) sequence identity to the amino acid sequence of SEQ ID NO: 61. In some embodiments, the anti-FcRn antibody is a full-length antibody. In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

For example, in some embodiments, there is provided a method of treating an individual having a disease or condition described above (e.g., autoimmune disorders and inflammatory diseases) comprising administering to the individual an effective amount of a pharmaceutical composition comprising an anti-FcRn antibody comprises a V_{H} comprising the amino acid sequence of SEQ ID NO: 50 or a variant thereof having at least about 80% (such as at least about any one of 80%, 85%, 90%, 95%, 96%, 97%, 98%, or 99%) sequence identity to the amino acid sequence of SEQ ID NO: 50, and a V_{L} comprising the amino acid sequence of SEQ ID NO: 61, or a variant thereof having at least about 80% (such as at least about any one of 80%, 85%, 90%, 95%, 96%, 97%, 98%, or 99%) sequence identity to the amino acid sequence of SEQ ID NO: 61. In some embodiments, the anti-FcRn antibody is a full-length antibody. In some embodiments, the full-length anti-FcRn antibody is an IgG1 or IgG4 antibody. In some embodiments, the disease or condition is selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome. In some embodiments, the individual is human.

In some embodiments, the anti-FcRn antibody provided herein is a full-length anti-FcRn antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 62. In some embodiments, the heavy chain constant region comprises or consists of the amino acid sequence of SEQ ID NO: 63. In some embodiments, the light chain constant region comprises or consists of the amino acid sequence of SEQ ID NO: 64. In some embodiments, the light chain constant region comprises or consists of the amino acid sequence of SEQ ID NO: 65.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or genetically prone to one or more of the diseases or disorders described herein (such as autoimmune disorders and inflammatory diseases). In some embodiments, the individual has one or more risk factors associated with one or more diseases or disorders described herein.

The present application in some embodiments provides a method of delivering an anti-FcRn antibody (such as any one of the anti-FcRn antibodies described herein, e.g., an isolated anti-FcRn antibody) to a cell producing FcRn in an individual, the method comprising administering to the individual a composition comprising the anti-FcRn antibody.

Many diagnostic methods for autoimmune disorders and inflammatory diseases or any other disease described above and the clinical delineation of those diseases are known in the art. Such methods include, but are not limited to, e.g., immunohistochemistry, PCR, and fluorescent in situ hybridization (FISH).

In some embodiments, the anti-FcRn antibodies (e.g., full-length anti-FcRn antibodies) and/or compositions of the application are administered in combination with a second, third, or fourth agent (including, e.g., a monoclonal antibody therapy for the treatment of an immune-mediated disease, an inhibitor of the complement system, immunosuppressant agents, immunomodulators, or a combination thereof) to treat diseases or disorders described herein.

In some embodiments, the therapeutic effect refers to the ameliorate, or prevent a targeted disease or condition, or to exhibit a detectable therapeutic or prophylactic effect. In some embodiment, the effect refers to the reduction of circulating IgG levels. In some embodiments, the reduction of circulating IgG levels can be restored over a period of about 1 or more weeks after the anti-FcRn antibody administration.

### Dosing and Method of Administering the Anti-FcRn Antibodies

The dose of the anti-FcRn antibody (such as isolated anti-FcRn antibody) compositions administered to an individual (such as a human) may vary with the particular composition, the mode of administration, and the type of disease being treated. In some embodiments, the amount of the composition (such as composition comprising isolated anti-FcRn antibody) is effective to result in an objective response (such as a partial response or a complete response) in the treatment of autoimmune disorders and inflammatory diseases. In some embodiments, the amount of the anti-FcRn antibody composition is sufficient to result in a complete response in the individual. In some embodiments, the amount of the anti-FcRn antibody composition is sufficient to result in a partial response in the individual. In some embodiments, the amount of the anti-FcRn antibody composition administered (for example when administered alone) is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the anti-FcRn antibody composition. Responses of an individual to the treatment of the methods described herein can be determined, for example, based on the reduction of circulating IgG levels.

In some embodiments, the amount of the composition (such as composition comprising isolated anti-FcRn antibody) is sufficient to control symptoms and reduce the risk of exacerbations of the individual. In some embodiments, the amount of the composition is sufficient to control symptoms and reduce the risk of exacerbations of the individual. In some embodiments, the amount of the composition (for example when administered along) is sufficient to produce clinical benefit of more than about any of 50%, 60%, 70%, or 77% among a population of individuals treated with the anti-FcRn antibody composition.

In some embodiments, the amount of the composition (such as composition comprising isolated anti-FcRn antibody), alone or in combination with a second, third, and/or fourth agent, is an amount sufficient to control symptoms and reduce the risk of exacerbations in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the anti-FcRn antibody (such as a full-length anti-FcRn antibody) in the composition is below the level that induces a toxicological effect (i.e., an effect above a clinically acceptable level of toxicity) or is at a level where a potential side effect can be controlled or tolerated when the composition is administered to the individual.

In some embodiments, the amount of the composition is close to a maximum tolerated dose (MTD) of the composition following the same dosing regimen. In some embodiments, the amount of the composition is more than about any of 80%, 90%, 95%, or 98% of the MTD.

In some embodiments, the amount of an anti-FcRn antibody (such as a full-length anti-FcRn antibody) in the composition is included in a range of about 0.001 μg to about 1000 μg.

In some embodiments of any of the above aspects, the effective amount of anti-FcRn antibody (such as a full-length anti-FcRn antibody) in the composition is in the range of about 0.1 μg/kg to about 100 mg/kg of total body weight.

The anti-FcRn antibody compositions can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal or transdermal. In some embodiments, sustained continuous release formulation of the composition may be used. In some embodiments, the composition is administered inhaled. In some embodiments, the composition is administered intravenously. In some embodiments, the composition is administered intraportally. In some embodiments, the composition is administered intraarterially. In some embodiments, the composition is administered intraperitoneally. In some embodiments, the composition is administered intrahepatically. In some embodiments, the composition is administered by hepatic arterial infusion. In some embodiments, the administration is to an injection site distal to a first disease site.

### Articles of Manufacture and Kits

In some embodiments of the application, there is provided an article of manufacture containing materials useful for the treatment of disease or condition described above, (e.g., autoimmune disorders and inflammatory diseases) or for delivering an anti-FcRn antibody (such as a full-length anti-FcRn antibody) to a cell producing FcRn of the individual. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an anti-FcRn antibody of the application. The label or package insert indicates that the composition is used for treating the particular condition. The label or package insert will further comprise instructions for administering the anti-FcRn antibody composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating disease or condition described above (such as autoimmune disorders and inflammatory diseases). In some embodiments, the package insert indicates that the composition is used for treating disease or condition selected from the group consisting of Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome.

Additionally, the article of manufacture may further comprise a second container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

Kits are also provided that are useful for various purposes, e.g., for treatment of disease or condition described above (e.g., autoimmune disorders and inflammatory diseases), or for delivering an anti-FcRn antibody (such as a full-length anti-FcRn antibody) to a cell expressing FcRn of the individual, optionally in combination with the articles of manufacture. Kits of the application include one or more containers comprising anti-FcRn antibody composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the application are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an anti-FcRn antibody (such as a full-length anti-FcRn antibody). In some embodiments, the kit comprises a) a composition comprising any one of the anti-FcRn antibodies described herein, and b) an effective amount of at least one other agent, wherein the other agent enhances the effects (e.g., treatment effect, detecting effect) of the anti-FcRn antibody. In some embodiments, the kit comprises a) a composition comprising any one of the anti-FcRn antibodies described herein, and b) instructions for administering the anti-FcRn antibody composition to an individual for treatment of a disease or condition described herein (e.g., autoimmune disorders and inflammatory diseases). In some embodiments, the kit comprises a) a composition comprising any one of the anti-FcRn antibodies described herein, b) an effective amount of at least one other agent, wherein the other agent enhances the effect (e.g., treatment effect, detecting effect) of the anti-FcRn antibody, and c) instructions for administering the anti-FcRn antibody composition and the other agent(s) to an individual for the treatment of a disease or condition described herein (e.g., autoimmune disorders and inflammatory diseases. The anti-FcRn antibody and the other agent(s) can be present in separate containers or in a single container. For example, the kit may comprise one distinct composition or two or more compositions wherein one composition comprises an anti-FcRn antibody and another composition comprises another agent.

In some embodiments, the kit comprises a nucleic acid (or set of nucleic acids) encoding an anti-FcRn antibody (such as a full-length anti-FcRn antibody). In some embodiments, the kit comprises a) a nucleic acid (or set of nucleic acids) encoding an anti-FcRn antibody, and b) a host cell for expressing the nucleic acid (or set of nucleic acids). In some embodiments, the kit comprises a) a nucleic acid (or set of nucleic acids) encoding an anti-FcRn antibody, and b) instructions for i) expressing the anti-FcRn antibody in a host cell, ii) preparing a composition comprising the anti-FcRn antibody, and iii) administering the composition comprising the anti-FcRn antibody to an individual for the treatment of a disease or condition described herein (e.g., autoimmune disorders and inflammatory diseases. In some embodiments, the kit comprises a) a nucleic acid (or set of nucleic acids) encoding an anti-FcRn antibody, b) a host cell for expressing the nucleic acid (or set of nucleic acids), and c) instructions for i) expressing the anti-FcRn antibody in the host cell, ii) preparing a composition comprising the anti-FcRn antibody, and iii) administering the composition comprising the anti-FcRn antibody to an individual for the treatment of a disease or condition described herein (e.g., autoimmune disorders and inflammatory diseases).

The kits of the application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the anti-FcRn antibody compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of an anti-FcRn antibody (such as a full-length anti-FcRn antibody) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the anti-FcRn antibody and pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this application. The application will now be described in greater detail by reference to the following non-limiting examples. The following examples further illustrate the application but, of course, should not be construed as in any way limiting its scope.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Generation of FcRn Polypeptides

This example illustrates the preparation of Neonatal Fc receptor (FcRn) polypeptide constructs used as antigens in eliciting and screening the anti-FcRn antibodies of the present disclosure.

FcRn is a heterodimer of MHC class I-like protein and 02-microglobulin, encoded by FCGRT and B2M, respectively. Human recombinant FCGRT & B2M Heterodimer Protein (hFcRn) was purchased from SinoBiological (cat #CT009-H08H). The coding sequence of the human FCGRT (Accession Number NP_001129491.1), human B2M (Accession Number NP_004039.1), Cynomolgus monkey FcRn-alpha (cynoFcRn, Accession Number NP_001271480.1), musculus FcRn-alpha (mFcRn, Accession Number NP_001344046.1), or Rattus norvegicus FcRn-alpha (rFcRn, Accession Number NP_203502.1) was synthesized, fused with His tag for purification and Avi tag for biotinylation. The amino acid sequences were provided in Table 5. These antigens were biotinylated using methods that are well known and commonly practiced by those skilled in the art.

TABLE 5

| SEQ | | |
|---|---|---|
| ID NO | Protein | Sequence |
| 66 | human FCGRT | AESHLSLLYHLTAVSSPAPGTPAFWVSGWLGPQQYLSYN |
| | | SLRGEAEPCGAWVWENQVSWYWEKETTDLRIKEKLFLE |
| | | AFKALGGKGPYTLQGLLGCELGPDNTSVPTAKFALNGEE |
| | | FMNFDLKQGTWGGDWPEALAISQRWQQQDKAANKELT |
| | | FLLFSCPHRLREHLERGRGNLEWKEPPSMRLKARPSSPGF |
| | | SVLTCSAFSFYPPELQLRFLRNGLAAGTGQGDFGPNSDGS |
| | | FHASSSLTVKSGDEHHYCCIVQHAGLAQPLRVELESPAK |
| | | SS |
| 67 | human B2M | MSRSVALAVLALLSLSGLEAIQRTPKIQVYSRHPAENGKS |
| | | NFLNCYVSGFHPSDIEVDLLKNGERIEKVEHSDLSFSKDW |
| | | SFYLLYYTEFTPTEKDEYACRVNHVTLSQPKIVKWDRDM |
| 68 | cynoFcRn | MRVPRPQPWALGLLLFLLPGSLGAESHLSLLYHLTAVSSP |
| | | APGTPAFWVSGWLGPQQYLSYDSLRGQAEPCGAWVWE |
| | | NQVSWYWEKETTDLRIKEKLFLEAFKALGGKGPYTLQG |
| | | LLGCELSPDNTSVPTAKFALNGEEFMNFDLKQGTWGGD |
| | | WPEALAISQRWQQQDKAANKELTFLLFSCPHRLREHLER |
| | | GRGNLEWKEPPSMRLKARPGNPGFSVLTCSAFSFYPPEL |
| | | QLRFLRNGMAAGTGQGDFGPNSDGSFHASSSLTVKSGDE |
| | | HHYCCIVQHAGLAQPLRVELETPAKSSVLVVGIVIGVLLL |
| | | TAAAVGGALLWRRMRSGLPAPWISLRGDDTGSLLPTPGE |
| | | AQDADSKDINVIPATA |
| 69 | mFcRn | SETRPPLMYHLTAVSNPSTGLPSFWATGWLGPQQYLTYN |
| | | SLRQEADPCGAWMWENQVSWYWEKETTDLKSKEQLFL |
| | | EALKTLEKILNGQKRGTYTLQGLLGCELASDNSSVPTAV |
| | | FALNGEEFMKFNPRIGNWTGEWPETEIVANLWMKQPDA |
| | | ARKESEFLLNSCPERLLGHLERGRRNLEWKEPPSMRLKA |
| | | RPGNSGSSVLTCAAFSFYPPELKFRFLRNGLASGSGNCST |
| | | GPNGDGSFHAWSLLEVKRGDEHHYQCQVEHEGLAQPLT |
| | | VDLDSSARSS |
| 70 | rFcRn | AEPRLPLMYHLAAVSDLSTGLPSFWATGWLGAQQYLTY |
| | | NNLRQEADPCGAWIWENQVSWYWEKETTDLKSKEQLF |
| | | LEAIRTLENQINGTFTLQGLLGCELAPDNSSLPTAVFALN |
| | | GEEFMRFNPRTGNWSGEWPETDIVGNLWMKQPEAARKE |
| | | SEFLLTSCPERLLGHLERGRQNLEWKEPPSMRLKARPGN |
| | | SGSSVLTCAAFSFYPPELKFRFLRNGLASGSGNCSTGPNG |
| | | DGSFHAWSLLEVKRGDEHHYQCQVEHEGLAQPLTVDLD |
| | | SPARSS |

The coding sequence was sub-cloned into the expression vector and expressed in Expi293 cells according to the manufacturer's protocol. Briefly, 293 cells were transfected with the expression vector respectively, and the cells were cultured at 37° C., under 8% CO₂ and 120 rpm for 5 days. For purification of His-tag protein, after harvest, the clarified supernatant media were loaded into a Histrap column equilibrated with 20 mM Sodium Phosphate buffer (pH7.4) containing 0.25M NaCl and 5 mM imidazole (pH8.0). The column was washed with 20 mM Sodium Phosphate buffer (pH7.4) containing 0.25M NaCl and 15 mM imidazole (pH8.0), then eluted with 20 mM Sodium Phosphate buffer (pH7.4) containing 0.25M NaCl and 100 mM imidazole (pH8.0).

### Example 2: Generation of Mouse Anti-FcRn Antibodies, Screening and Characterization

This example illustrates the methods of generating mouse anti-FcRn antibodies, and methods to screen and select antibodies for further characterization.

Immunization and primary selection: Balb/c mice were immunized with recombinant human FCGRT adjuvanted with Freund's adjuvant. Endpoint titers were determined by ELISA, and the spleens and lymph nodes were harvested. A phage display library was then constructed according to standard protocols. Briefly, RNA collected from the highest titer mouse spleen was reverse-transcribed into cDNA, and the V_{H} and V_{κ} fragments were amplified using V_{H}- and V_{K}-specific primers. Upon gel extraction and purification, scFvs were generated by linking V_{H} and V_{K}, and were cloned into the phage display vector pDAN5 using SfiI. Upon ligation, the vector was used to transduce TG1 phage display electroporation-competent cells to obtain the phage scFv antibody display library. ScFv antibodies specific to FcRn were isolated from the phage display library in a series of repeated selection cycles. Briefly, phage scFv library (2×10¹¹ PFU) was added to biotinylated human FCGRT, and incubated for 2 hours at 37° C. Human FCGRT with phage bound was captured on streptavidin coated magnetic beads. Unbound phages were washed away. After washing with TBST 8-15 times (increasing number of washes for every round of selection), phages that specifically bound to human FCGRT were washed off with Glycine-HCl (pH2.2). These phages were used to transduce TG1 cells in the log phase, with the addition of Ampicillin, and cultured for 1 hour. Upon the addition of helper phage, the cells were cultured on a rocking bed overnight at 200 rpm at 28° C. Culture media was collected the next day, centrifuged to obtain the supernatant, and subjected to the next round of selection. A panel of positive scFv antibodies were obtained at the end of the primary selection process.

Human IgG blocking assay: The positive antibodies identified in the primary selection were further tested for their ability to block the human IgG binding to human FcRn. 96-well ELISA plates were coated with human IgG (Jackson ImmunoResearch, cat #009-000-003) overnight at 4° C. After removing the coating solution, a blocking buffer containing 1% BSA was added to the plates followed by incubation at room temperature for 1 hour to block unspecific binding. Plates were then washed with wash buffer. Biotinylated human FCGRT and the supernatant of the phages obtained above were added to the well and incubated at room temperature for 1 hour. After incubation, 100 μL of SA-HRP (1:20,000) was added to each well and incubated for 1 hour at 37° C. The wells were washed before adding 50p/well of TMB and incubated for 5-10 minutes at 37° C. 2M H₂SO₄ was used to stop the reaction. The ELISA results (OD450) were analyzed, and the binding curves were generated by PRISM.

### Example 3: Generation and Characterization of Full-Length Chimeric Anti-FcRn Antibodies

Generation of full-length chimeric anti-FcRn antibodies: The most potent clones were reformatted as human IgG1 or IgG4 antibody molecules with a human IgG1 or IgG4 heavy chain constant domain, and a human kappa or lambda light chain constant domain. Briefly, V_{L} was amplified and introduced into eukaryotic expression vectors pTT5-K (containing kappa constant domain) or pTT5-L (containing lambda constant domain), and V_{H} was introduced into pTT5-H1 (containing IgG1 heavy chain constant domain), or pTT5-H4 (containing IgG4 heavy chain constant domain), respectively. Plasmids expressing the light or heavy chains were extracted and used to co-transfect 293F cells. After the cells were cultured at 37° C., 8% CO₂ and 120 rpm for 5 days, the culture media was purified using Protein A affinity chromatography. Briefly, after harvest, the clarified supernatant media were mixed with protein A resin equilibrated with PBS buffer and incubated with gentle rotation for 1.5 h at room temperature. After incubation, the slurry was loaded into a column and the resin was washed with PBS buffer containing 0.15M NaCl and then eluted with 50 mM sodium phosphate (pH 3.0). The variable domain sequences of anti-human FcRn antibodies were summarized in Tables 2A and 3A.

hFcRn binding of purified full-length chimeric antibodies: The hFcRn antigen binding ELISA was performed on the purified full-length chimeric antibodies (reformatted as IgG4). Briefly, plates were coated overnight at 4° C. with 1 μg/mL of hFcRn (cat #CT009-H08H) in PBS. After removing the coating solution, the wells were blocked by adding blocking buffer and incubated at room temperature for 1 hour. Plates were then washed 6 times with wash buffer. Serial dilutions of purified antibodies in PBS were added to individual wells followed by incubation at room temperature for 1 hour. Plates were washed 6 times with wash buffer. Then 100 μL/well goat anti-human IgG Fab-HRP (Jackson ImmunoResearch cat #109-036-097) at a 1:8000 dilution was added. Plates were incubated at 37° C. for 1 hour, washed 6 times with wash buffer, and developed for 5-10 minutes with 50 L/well of TMB substrate, then quenched with 2M H₂SO₄. Rozanolixizumab was used as a reference antibody and FY38 (an antibody that does not bind to FcRn, made by Staidson Co., Ltd) was used as a negative control. The ELISA results (OD450) were analyzed, and the binding curves were generated by Graphpad PRISM. As shown in FIG. 1, the chimeric FcRn antibodies did bind to hFcRn, and the EC50 values for hFcRn binding were shown in Table 6.

TABLE 6

| Antibody | hFcRn binding EC50 (nM) |
|---|---|
| ZLP22 | 2.389 |
| ZLP193 | 0.4394 |
| ZLP64 | 0.2122 |
| ZLP42 | 0.9810 |
| ZLP44 | 0.6734 |
| Rozanolixizumab | 1.037 |

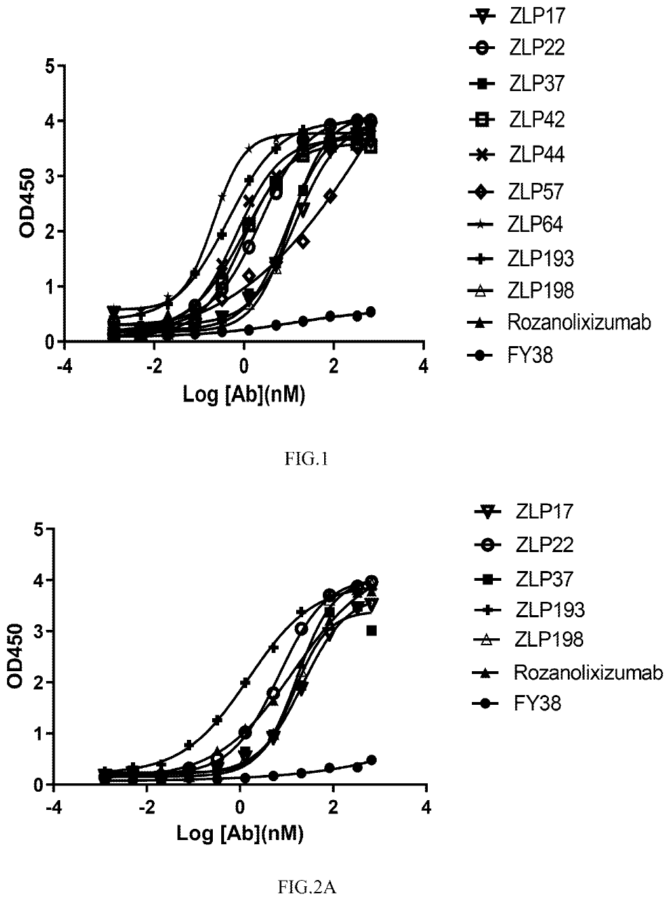

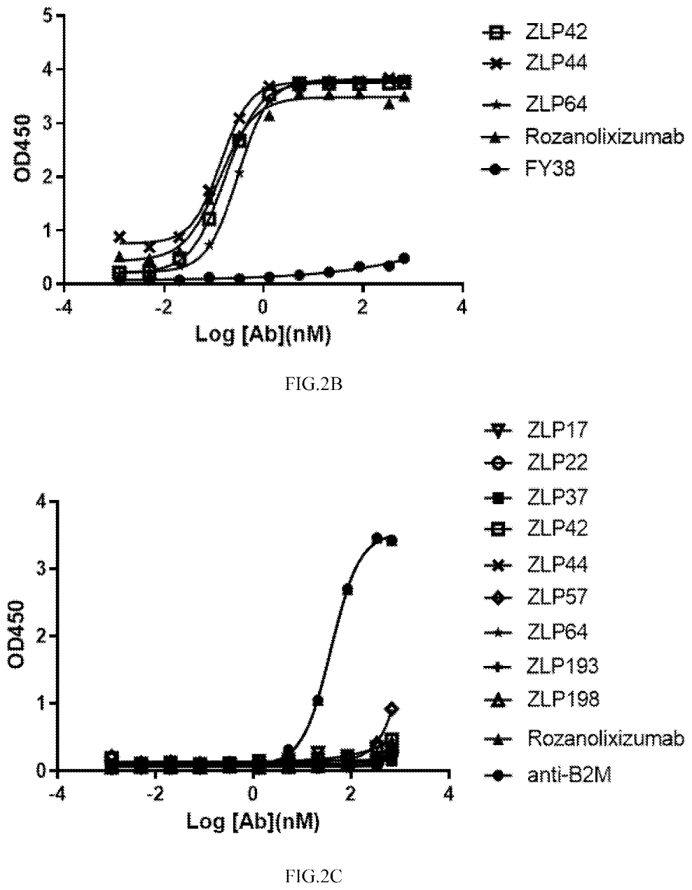

Measurement of the binding specificity of anti-FcRn antibodies to FCGRT and B2M by EILSA: Briefly, human FCGRT or B2M was coated on a 96-well plate at 4° C. overnight, after removing the coating solution, a blocking buffer containing 1% BSA was added into the plates followed by incubation at room temperature for 1 hour to block unspecific binding. Plates were then washed with wash buffer, and serial dilutions of anti-FcRn antibodies were added to the wells and incubated at room temperature for 1 hour. After washing, 100 μL of goat anti-human IgG Fab-HRP at a 1:8000 dilution was added to each well and incubated for 1 hour at 37° C. The wells were washed before adding 100 μL/well of TMB and incubated for 5-10 minutes at 37° C. 2M H₂SO₄ was used to stop the reaction. Anti-B2M antibody (SinoBiological, cat #11976-MM35) was used as a positive control in B2M binding assay. FY38 was used as a negative control in FCGRT binding assay. Rozanolixizumab was used as a reference antibody. The ELISA results (OD450) were analyzed, and the binding curves were generated by Graphpad PRISM. As shown in FIG. 2A-2C, the chimeric anti-FcRn antibodies bind to FCGRT of human FcRn protein (FIG. 2A-2B) but not to B2M (FIG. 2C).

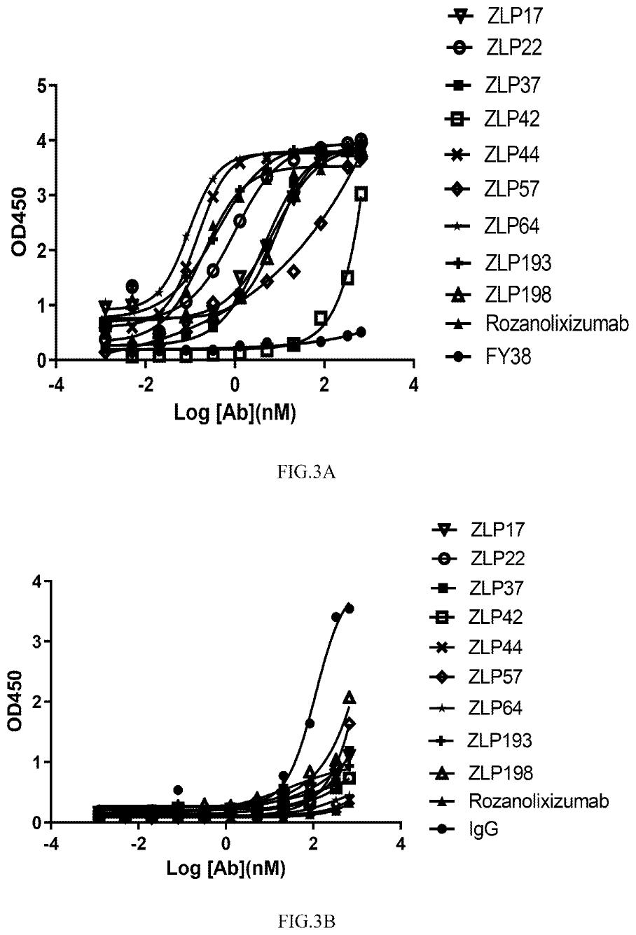

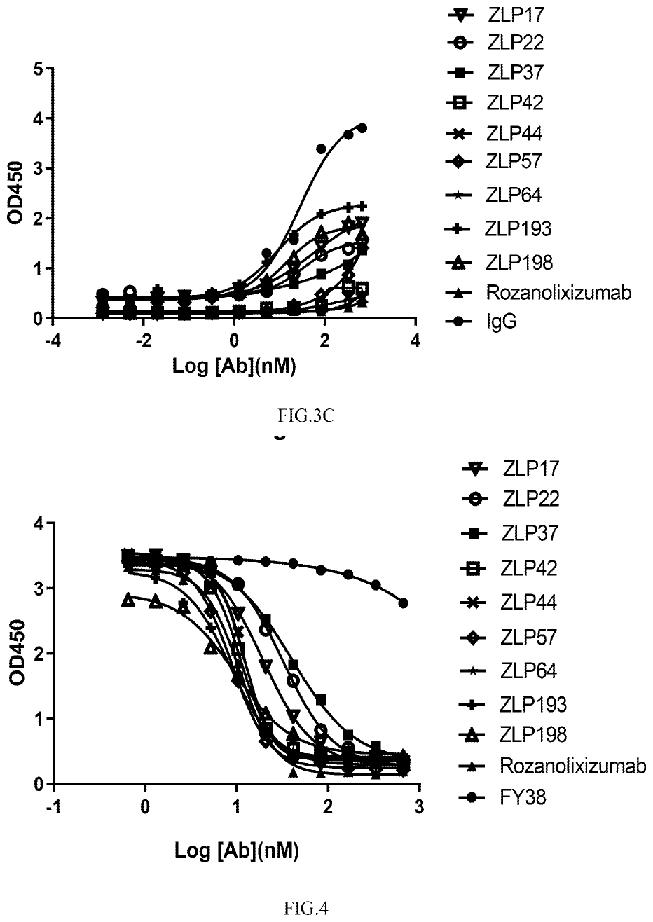

Analysis of cross-reactivity to FcRn from different species: To examine whether the anti-FcRn antibodies bind to FcRn from different species, antigen binding ELISA assays were performed as described above except that cynoFcRn, mFcRn, or rFcRn was used in the assay instead of hFcRn. Human IgG (Jackson ImmunoResearch, cat #009-000-003) was used as a positive control. As shown in FIG. 3A-3C, the chimeric FcRn antibodies did bind to cynoFcRn (FIG. 3A), but did not bind to rFcRn (FIG. 3B) and showed extremely weak binding to mFcRn (FIG. 3C).

Human IgG blocking assay: the chimeric anti-FcRn antibodies were tested for their ability to block human IgG binding to human FcRn. Human IgG (Jackson ImmunoResearch, cat #009-000-003) was coated on 96-well ELISA plates overnight at 4° C. After removing the coating solution, the wells were blocked with a blocking buffer containing 1% BSA at room temperature for 1 hour. Plates were then washed with wash buffer. Biotinylated human FCGRT and the chimeric anti-FcRn antibodies were added to the well and incubated at room temperature for 1 hour. After incubation, 100 μL of SA-HRP (1:20,000) was added to each well and incubated for 1 hour at 37° C. The wells were washed before adding 100 μL/well of TMB and incubated for 5-10 minutes at 37° C. 2M H₂SO₄ was used to stop the reaction. Rozanolixizumab was used as a reference antibody, and FY38 was used as a negative control. The ELISA results (OD450) were analyzed, and the binding curves were generated by PRISM. As shown in FIG. 4, the chimeric anti-FcRn antibodies effectively competed with human IgG and inhibited the binding of human IgG to human FCGRT as determined by EILSA. The IC50 values for IgG blocking was shown in Table 7.

TABLE 7

| Antibody | IgG blocking IC50 (nM) |
|---|---|
| ZLP193 | 8.696 |
| ZLP57 | 8.885 |
| ZLP44 | 12.26 |
| ZLP42 | 10.94 |
| ZLP64 | 12.52 |
| ZLP198 | 9.760 |
| Rozanolixizumab | 10.90 |

IgG recycling assay: FcRn expression is primarily intracellular (Borvak J et al. 1998, Int. Immunol., 10 (9) 1289-98 and Cauza K et al. 2005, J. Invest. Dermatol., 124 (1), 132-139), and associated with endosomal and lysosomal membranes. The Fc portion of IgG binds to FcRn at acidic pH (<6.5), but not at a neutral physiological pH (7.4) (Rhagavan M et al. 1995) and this pH-dependency facilitates the recycling of IgG. Once it is taken up by pinocytosis and enters the acidic endosome, IgG bound to FcRn will be recycled along with the FcRn to the cell surface, whereas at the physiologically neutral pH the IgG will be released. (Ober R J et al. 2004, The Journal of Immunology, 172, 2021-2029). Any IgG not bound to FcRn will enter the lysosomal degradative pathway.

This assay was performed as described in (Grevys A, et al. A human endothelial cell-based recycling assay for screening of FcRn targeted molecules. Nat Commun. 2018 Feb. 12; 9(1):621; Smith B, et al. Generation and characterization of a high affinity anti-human FcRn antibody, rozanolixizumab, and the effects of different molecular formats on the reduction of plasma IgG concentration. MAbs. 2018 October; 10(7):1111-1130), Madin-Darby Canine Kidney (MDCK) cells (ATCC, CCL-34™) had been stably transfected with a human FCGRT and B2M double gene vector. A stable MDCK cell clone that was able to recycle human IgG was selected. MDCK cells were seeded in a 96-well plate until being qualified for the experiment, the complete cell culture solution was removed. After washing with HBSS buffer containing 1% BSA (pH 7.4), 100 μl of HBSS buffer containing 1% BSA (pH 7.4) was added to each well and incubated for 20 min. Serial dilution of anti-FcRn antibodies was added to individual well followed by incubation at 37° C. with 5% CO₂ for 1 hour. After washing with HBSS buffer containing 1% BSA (pH 5.9), biotinylated human IgG (Jackson ImmunoResearch cat #009-060-003) was added to individual well followed by incubation at 37° C. with 5% CO₂ for 1 hour. After washing with HBSS buffer containing 1% BSA (pH 5.9), 100 μl of HBSS buffer containing 1% BSA (pH 7.4) was added to individual well followed by incubation at 37° C. with 5% CO₂ for 2 hours, which allows release of surface-exposed, bound IgG into the supernatant. Then the MDCK cell supernatant was collected.

Another 96-well plate was coated with goat anti-human IgG Fc antibody overnight at 4° C., after removing the coating solution, the plates were blocked by the addition of blocking solution containing 1% BSA, and incubated at room temperature for 1 hour. After washing the plate, the MDCK cell supernatant was added into the coated 96-well plate, followed by incubation at 37° C. for 1.5 hours. After that, SA-HRP was added and incubated at 37° C. for 1 hour. The wells were washed before adding 50p/well of TMB and incubated for 5-10 minutes at 37° C. 2M H₂SO₄ was used to stop the reaction. Rozanolixizumab was used as a reference antibody, and FY38 was used as a negative control. The total IgG ELISA results (OD450) were analyzed, and the inhibition curves were generated by PRISM.

As shown in FIG. 5, the anti-FcRn antibodies effectively inhibited the recycling of human IgG. The IC50 values for IgG recycling were shown in Table 8.

TABLE 8

| Antibody | IgG Recycle IC50 (nM) |
|---|---|
| ZLP22 | 5.007 |
| ZLP17 | 3.236 |
| ZLP37 | 3.125 |
| ZLP193 | 10.07 |
| ZLP64 | 10.71 |
| ZLP44 | 2.503 |
| Rozanolixizumab | 7.801 |

Effects of the chimeric anti-FcRn antibodies in inhibiting HSA binding to FcRn: the long circulating half-life of human serum albumin (HSA), the most abundant protein in mammalian plasma, derives from pH-dependent endosomal salvage from degradation, mediated by FcRn. And HSA binds many endogenous and exogenous molecules, such as fatty acids (FAs) (Schmidt M M, et al. Crystal structure of an HSA/FcRn complex reveals recycling by competitive mimicry of HSA ligands at a pH-dependent hydrophobic interface. Structure. 2013 Nov. 5; 21(11):1966-78). Commercial HSA (Chengdu Rongsheng Pharmaceutical, S10940024) was coated on 96-well plates at 4° C. overnight. After removing the coating solution, a blocking buffer containing 1% BSA was added to the plates followed by incubation at room temperature for 1 hour to block unspecific binding. Plates were then washed with wash buffer, biotinylated human FCGRT, and serial dilutions of the anti-FcRn antibodies were added to the well and incubated at room temperature for 1 hour. Palmitic acid (PA), one kind of fatty acids, was used as a positive control. After incubation, 100 μL of SA-HRP (1:20,000) was added to each well and incubated for 1 hour at 37° C. The wells were washed before adding 100 μL/well of TMB and incubated for 5-10 minutes at 37° C. 2M H₂SO₄ was used to stop the reaction. The ELISA results (OD450) were analyzed, and the inhibition curves were generated by Graphpad PRISM. The chimeric anti-FcRn antibodies ZLP193, ZLP37, ZLP17, ZLP22, ZLP198, ZLP57, ZLP44, ZLP64 and ZLP42 did not inhibit the binding of HSA to FcRn (data not shown).

### Example 4: Preparation of Humanized Versions of ZLP193

This example illustrates the preparation of humanized versions of the anti-FcRn antibody ZLP193.

Humanization of anti-FcRn antibody: The light chain variable region (V_{L}) and heavy chain variable region (V_{H}) sequences of the antibody from ZLP193 were aligned to human germline antibody sequences respectively. The human germline kappa light chain (Gene ID—V gene: IGKV3-11*01) and heavy chain (Gene ID—V gene: IGHV4-38-2*01) were used as the human frameworks.

The complementarity-determining regions (CDRs) of murine FcRn antibody light chain and heavy chain were grafted into the identified closest human frameworks respectively to generate humanized antibody clone. In this process, antibody ZLP193 was humanized by grafting the CDRs from the murine antibody V-regions onto human germline antibody V-region frameworks, the CDRs grafted from the donor to the acceptor sequence are as defined by Kabat (Kabat et al., 1987). In order to recover the activity of the antibody, some framework residues from the murine V-regions that were found to be parts of V_{H}-V_{L} interacting interface or the framework residues acting as “Vernier” zone, which may adjust CDR structure and fine-tune to fit antigen (Foote et al., 1992) were also retained in the humanized sequence.

The variable domain sequences of the humanized antibodies (named ZLP1-3-2, ZLP1-3-2F and ZLP1-3-2M) were summarized in Tables 2B and 3B.

### Example 6: In Vitro Assays of Humanized Anti-FcRn Antibodies

Generation of recombinant IgG versions of humanized anti-FcRn antibodies

The full-length IgG versions of humanized FcRn antibodies (reformatted as IgG4) were generated as described in Example 3.

FcRn binding ELISA assay: the binding ability of the humanized anti-FcRn antibodies ZLP1-3-2, ZLP1-3-2F and ZLP1-3-2M to hFcRn or cynoFcRn was measured by ELISA as described in Example 3. FY38 was used as a negative control.

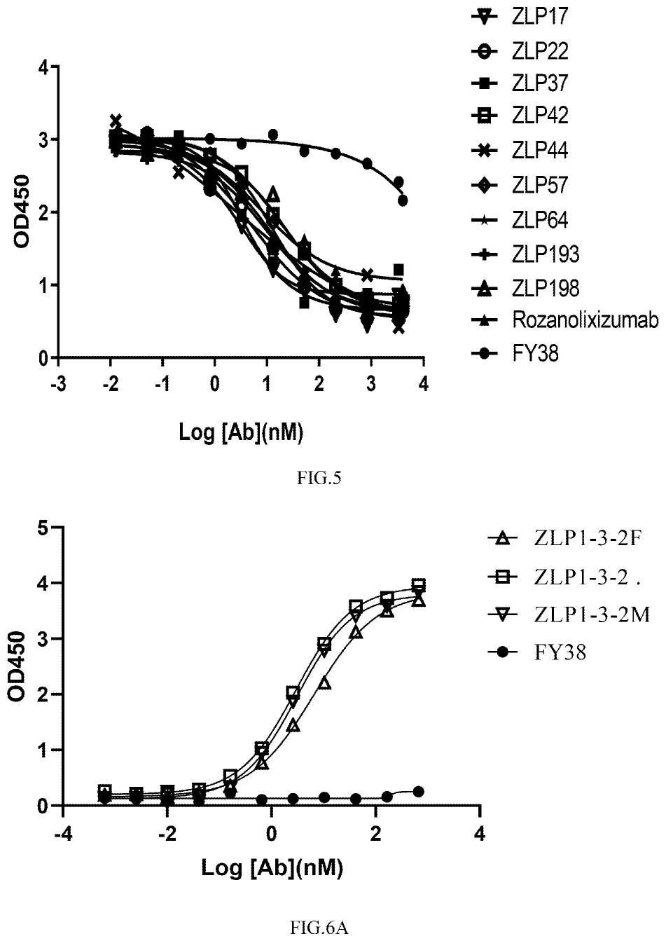

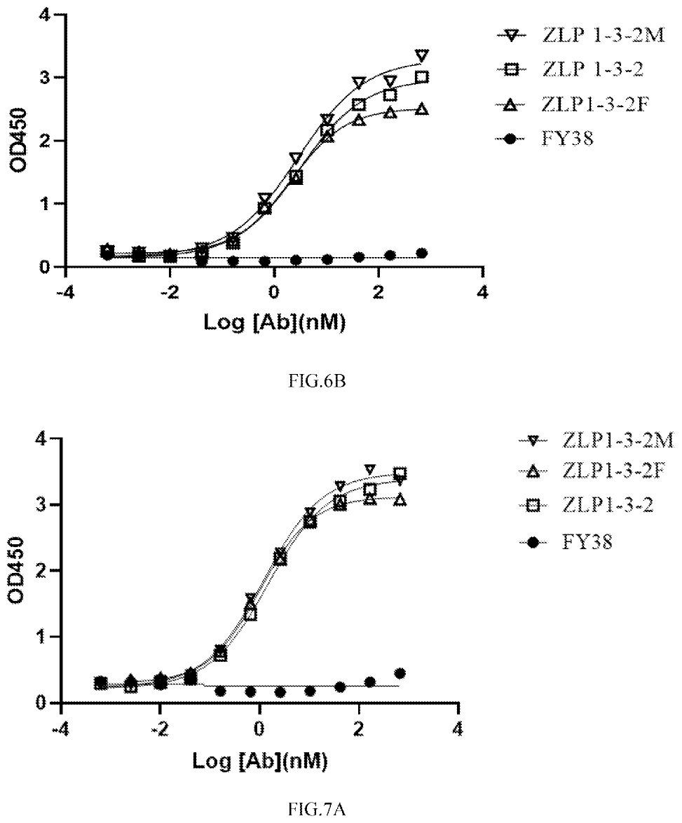

As shown in FIG. 6A-6B, the humanized antibodies could bind to hFcRn (FIG. 6A) or cynoFcRn (FIG. 6B) with high affinity. The EC50 values were shown in Table 9.

TABLE 9

| | hFcRn binding | cynoFcRn binding |
|---|---|---|
| Antibody | EC50 (nM) | EC50 (nM) |
| ZLP1-3-2 | 2.953 | 3.125 |
| ZLP1-3-2M | 3.110 | 2.822 |
| ZLP1-3-2F | 6.629 | 2.057 |

Measurement of the binding specificity of anti-FcRn antibodies to FCGRT and B2M by EILSA: this assay was performed as the method described in Example 3, FY38 was used as a negative control.

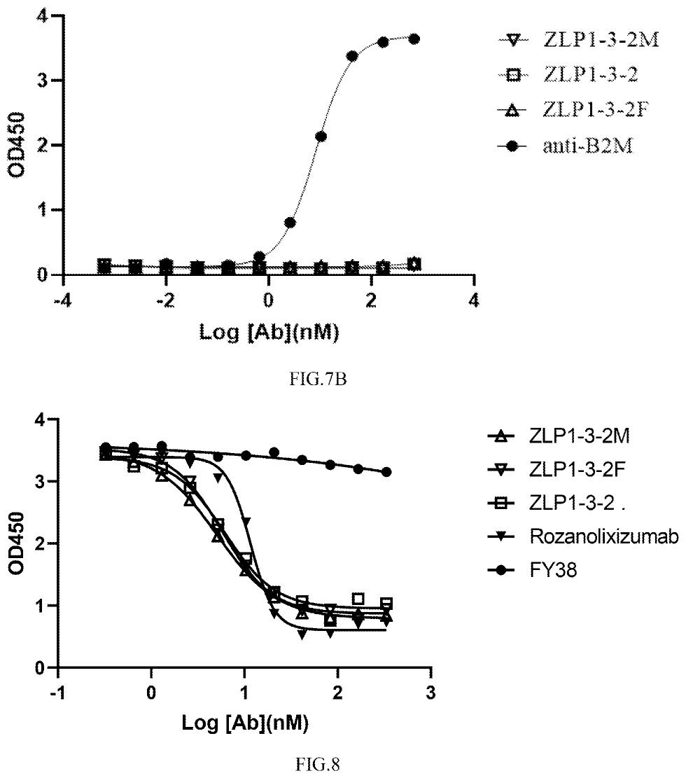

As shown in FIG. 7A-7B, the humanized anti-FcRn antibodies ZLP1-3-2, ZLP1-3-2F and ZLP1-3-2M bind to FCGRT of human FcRn protein (FIG. 7A) but not to B2M (FIG. 7B). The EC50 values for FCGRT binding were shown in Table 10.

TABLE 10

| Antibody | FCGRT binding EC50 (nM) |
|---|---|
| ZLP1-3-2 | 1.503 |
| ZLP1-3-2M | 1.217 |
| ZLP1-3-2F | 1.025 |

Determination of humanized anti-FcRn antibodies binding kinetics: The binding affinity of anti-FcRn antibodies ZLP1-3-2F and ZLP1-3-2M (reformatted as human IgG4) were characterized using Biacore T200 (GE). The anti-FcRn antibodies were stabilized on sensor chip CM5. The affinities for hFcRn of antibodies at various concentrations were measured. The range of concentrations included 10, 5, 2.5, 1.25, 0.625, 0.3125, 0.15625, 0.078, 0.039, 0.0195, and 0 nm. Using the SPR technology, the association and dissociation rates were measured, and binding affinity was determined. Table 11 shows the Kon, Koff, and Kd of ZLP1-3-2F and ZLP1-3-2M at pH6.0.

TABLE 11

| Antibody | Kon(1/Ms) | Koff(1/s) | Kd(M) |
|---|---|---|---|
| ZLP1-3-2F | 3.285E+6 | 1.999E−3 | 6.09E−10 |
| ZLP1-3-2M | 3.732E+6 | 1.84E−3 | 4.93E−10 |

Human IgG blocking assay: the humanized anti-FcRn antibodies ZLP1-3-2F and ZLP 1-3-2M were tested for the ability to block human IgG binding to human FCGRT as described in Example 3. Rozanolixizumab was used as a reference antibody, and FY38 was used as a negative control.

As shown in FIG. 8, the binding of human IgG to human FCGRT was inhibited by each of the exemplary anti-FcRn antibodies, demonstrating the ability of the humanized anti-FcRn antibodies to effectively compete with human IgG and inhibit the binding of human IgG to human FCGRT, and the anti-FcRn antibodies exhibited better efficacy as compared to the reference antibody Rozanolixizumab. The IC50 values for IgG blocking were shown in Table 12.

TABLE 12

| Antibody | IgG blocking IC50 (nM) |
|---|---|
| ZLP-1-3-2 | 6.16 |
| ZLP-1-3-2M | 5.12 |
| ZLP-1-3-2F | 5.77 |
| Rozanolixizumab | 11.62 |

IgG recycling assay: the humanized anti-FcRn antibodies ZLP1-3-2, ZLP1-3-2F and ZLP 1-3-2M were tested for the ability to block the recycling of human IgG as described in Example 3. Rozanolixizumab was used as a reference antibody, and FY38 was used as a negative control.

As shown in FIG. 9, the humanized anti-FcRn antibodies inhibited the recycling of human IgG and exhibited better or comparable efficacy to inhibit the IgG recycling when compared with the reference antibody Rozanolixizumab. The IC50 values for inhibiting IgG recycling were shown in Table 13.

TABLE 13

| Antibody | IgG Recycle IC50 (nM) |
|---|---|
| ZLP1-3-2 | 4.038 |
| ZLP1-3-2M | 11.86 |
| ZLP1-3-2F | 8.358 |
| Rozanolixizumab | 11.83 |

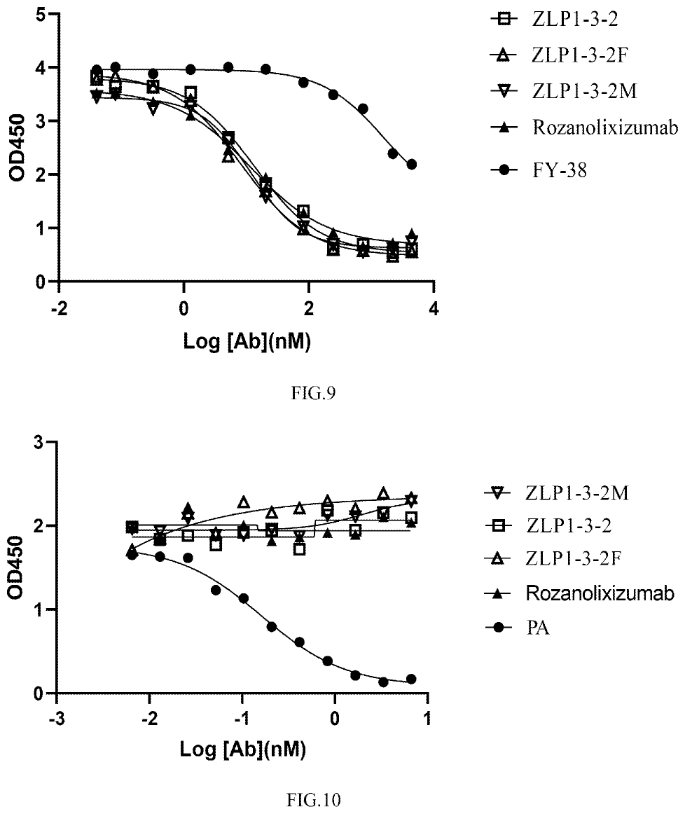

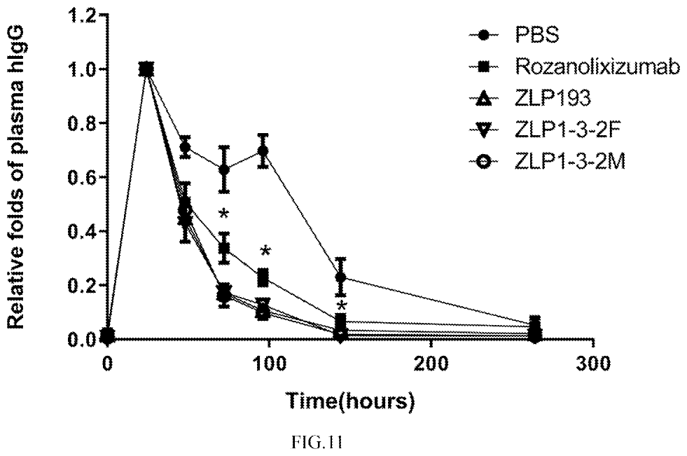

HSA-FcRn binding assay: Effects of the humanized anti-FcRn antibodies ZLP1-3-2, ZLP1-3-2F and ZLP 1-3-2M in inhibiting HSA binding to FcRn were tested as described in Example 3. Palmitic acid (PA) was used as a positive control. As shown in FIG. 10, the humanized anti-FcRn antibodies did not inhibit the binding of HSA to FcRn.

### Example 7: Pharmacodynamics (PD) of Anti-FcRn Antibodies

PD studies in the mouse: an in vivo study using transgenic mice expressing human FCGRT were conducted to examine the effects of anti-FcRn antibodies on human IgG clearance. Transgenic mice expressing human FcRn were conducted to examine the pharmacodynamics activity of the anti-FcRn antibodies on human IgG clearance. Healthy adult transgenic mice (C57BL/6-Fcgrt^{tm1(hFegtg)}/Bcgen, Beijing Biocytogen Co., Ltd) were used in this assay, with 6 mice/group. At time point 0 h, all mice were pre-injected intravenously with human IgG at a dose of 500 mg/kg. At time point 24 h, i.e. 24 hours after injection of human IgG, the mice were injected with ZLP193 (reformatted as IgG4), ZLP1-3-2M (reformatted as IgG4), ZLP1-3-2F (reformatted as IgG4) or Rozanolixizumab at a dose of 30 mg/kg, respectively. Rozanolixizumab was used as a reference antibody, and PBS buffer was used as a negative control. Blood samples were collected from each animal at time point 0 h (before injection of human IgG), time point 24 h, (before injection of anti-FcRn antibody), and subsequently at time point 48 h, 72 h, 96 h, 144 h, and 264 h after human IgG injection. After centrifugation, the plasma was used for analyzing the human IgG concentration using ELISA. The serum IgG level at 24 h after human IgG injection was set as 1.0 (100%), then the relative folds of serum IgG level were calculated.

The results in FIG. 11 showed that the chimeric anti-FcRn antibody ZLP193 and humanized anti-FcRn antibodies ZLP1-3-2M and ZLP1-3-2F were able to decrease human IgG (abbreviation hIgG in FIG. 11) rapidly in mice after intravenous injection. And at time point 48 h, i.e. 24 hours after anti-FcRn antibody injection, the serum IgG level in all the 3 groups (ZLP193, ZLP1-3-2M and ZLP1-3-2F) had decreased to about 0.4-0.5 fold relative to 100% serum IgG level, while in the PBS control group, it had only decreased to about 0.7 fold; at time point 72 h, i.e. 48 hours after antibody injection, the serum IgG level in all the 3 groups had decreased to about 0.2 fold, while it in the PBS control group still retained about 0.6-0.7 fold relative to 100% serum IgG level; at time point 96 h, i.e. 72 hours after antibody injection, the serum IgG level in all the 3 groups had decreased to about 0.1 fold, while the level in the PBS control group still retained about 0.7 fold relative to 100% serum IgG level. In summary, the PD assay results turned out that ZLP193, ZLP1-3-2M and ZLP1-3-2F had good efficacy to degrade serum IgG in vivo, and had significantly better pharmacodynamics activity as compared to the reference antibody Rozanolixizumab (P<0.01).

1. (canceled)

2. (canceled)

3. An isolated anti-FcRn antibody, wherein the anti-FcRn antibody comprises:
a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 15; and a V_{L} comprising an LC-CDR1 comprising the amino acid sequence of SEQ ID NO: 23, an LC-CDR2 comprising the amino acid sequence of SEQ ID NO: 29, and an LC-CDR3 comprising the amino acid sequence of SEQ ID NO: 34.

4. (canceled)

5. The isolated anti-FcRn antibody of claim 3, comprising:
(i) a V_{H} comprising the amino acid sequence of SEQ ID NO: 41, or a variant thereof having at least about 80% sequence identity to SEQ ID NO: 41 and a V_{L} comprising the amino acid sequence of SEQ ID NO: 52, or a variant thereof having at least about 80% sequence identity to SEQ ID NO: 52;
(ii) a V_{H} comprising the amino acid sequence of SEQ ID NO: 50, or a variant thereof having at least about 80% sequence identity to SEQ ID NO: 50 and a V_{L} comprising the amino acid sequence of SEQ ID NO: 60, or a variant thereof having at least about 80% sequence identity to SEQ ID NO: 60;
(iii) a V_{H} comprising the amino acid sequence of SEQ ID NO: 51, or a variant thereof having at least about 80% sequence identity to SEQ ID NO: 51 and a V_{L} comprising the amino acid sequence of SEQ ID NO: 61, or a variant thereof having at least about 80% sequence identity to SEQ ID NO: 61; or
(iv) a V_{H} comprising the amino acid sequence of SEQ ID NO: 50, or a variant thereof having at least about 80% sequence identity to SEQ ID NO: 50 and a V_{L} comprising the amino acid sequence of SEQ ID NO: 61, or a variant thereof having at least about 80% sequence identity to SEQ ID NO: 61.

6.-11. (canceled)

12. The isolated anti-FcRn antibody of claim 3, wherein the anti-FcRn antibody binds to the FcRn with a Kd from about 0.1 pM to about 1 nM.

13. The isolated anti-FcRn antibody according to claim 3, wherein the anti-FcRn antibody comprises an Fc fragment.

14. The isolated anti-FcRn antibody of claim 13, wherein the anti-FcRn antibody is a full-length IgG antibody.

15. The isolated anti-FcRn antibody of claim 14, wherein the anti-FcRn antibody is a full-length IgG1, IgG2, IgG3 or IgG4 antibody.

16. The isolated anti-FcRn antibody of claim 3, wherein the anti-FcRn antibody is chimeric, human, or humanized.

17. The isolated anti-FcRn antibody according to claim 3, wherein the anti-FcRn antibody is an antigen binding fragment selected from the group consisting of a Fab, a Fab′, a F(ab)′2, a Fab′-SH, a single-chain Fv (scFv), an Fv fragment, a dAb, a Fd, or a diabody.

18. An isolated nucleic acid molecule that encodes the isolated anti-FcRn antibody according to claim 3.

19. A vector comprising the nucleic acid molecule of claim 18.

20. An isolated host cell comprising the isolated nucleic acid molecule of claim 18, or a vector comprising the nucleic acid molecule of claim 18.

21. A method of producing an isolated anti-FcRn antibody, comprising:
a) culturing the host cell of claim 20 under conditions effective to express the anti-FcRn antibody; and
b) obtaining the expressed anti-FcRn antibody from the host cell.

22. A pharmaceutical composition comprising the anti-FcRn antibody according to claim 3, and a pharmaceutically acceptable carrier.

23. A method of treating a disease or condition in an individual in need thereof, comprising administering to the individual an effective amount of the pharmaceutical composition of claim 22.

24. The method of claim 23, wherein the disease or condition is an autoimmune disorders or inflammatory diseases.

25. The method of claim 24, wherein the disease or condition is selected from Myasthenia Gravis (MG), Pemphigus vulgaris, Neuromyelitis optica, Guillain-Barre syndrome, lupus, Idiopathic Thrombocytopenia Purpura (ITP), thrombotic thrombocytopenic purpura. rheumatoid arthritis (RA), systemic lupus erythematosus (SLE), Grave's Disease, autoimmune myocarditis, Membrane Glomerulonephritis, diabetes mellitus, Type I diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, sarcoidosis, polymyositis, polyarteritis, cutaneous vasculitis, pemphigus, pemphigoid, Goodpasture's syndrome, Kawasaki's disease, systemic sclerosis, anti-phospholipid syndrome, and Sjogren's syndrome.


SEQUENCE LISTING (70 sequences)

SEQ ID NO: 1 - protein, 6 aa, synthetic construct
  REGION 1..6: note = Synthetic Construct
SGYSWH

SEQ ID NO: 2 - protein, 5 aa, synthetic construct
  REGION 1..5: note = Synthetic Construct
SYAMQ

SEQ ID NO: 3 - protein, 5 aa, synthetic construct
  REGION 1..5: note = Synthetic Construct
SYTMH

SEQ ID NO: 4 - protein, 5 aa, synthetic construct
  REGION 1..5: note = Synthetic Construct
SYAMH

SEQ ID NO: 5 - protein, 5 aa, synthetic construct
  REGION 1..5: note = Synthetic Construct
SYNMY

SEQ ID NO: 6 - protein, 5 aa, synthetic construct
  REGION 1..5: note = Synthetic Construct
DTYMY

SEQ ID NO: 7 - protein, 16 aa, synthetic construct
  REGION 1..16: note = Synthetic Construct
YIHDSGGTNYNPSLKS

SEQ ID NO: 8 - protein, 17 aa, synthetic construct
  REGION 1..17: note = Synthetic Construct
YINPGTGYTNYNQKFKD

SEQ ID NO: 9 - protein, 17 aa, synthetic construct
  REGION 1..17: note = Synthetic Construct
YINPSSGYTNYNQKFKD

SEQ ID NO: 10 - protein, 16 aa, synthetic construct
  REGION 1..16: note = Synthetic Construct
YIHNSGSTNYNPSLKS

SEQ ID NO: 11 - protein, 16 aa, synthetic construct
  REGION 1..16: note = Synthetic Construct
YIHSSGSTNYNPSLKS

SEQ ID NO: 12 - protein, 17 aa, synthetic construct
  REGION 1..17: note = Synthetic Construct
YIDPYNGGTRYNQKFKG

SEQ ID NO: 13 - protein, 17 aa, synthetic construct
  REGION 1..17: note = Synthetic Construct
YIDPYNGGTNYNQKFKG

SEQ ID NO: 14 - protein, 17 aa, synthetic construct
  REGION 1..17: note = Synthetic Construct
RIDPANGITKFDPKFQD

SEQ ID NO: 15 - protein, 9 aa, synthetic construct
  REGION 1..9: note = Synthetic Construct
EENYRYFDV

SEQ ID NO: 16 - protein, 10 aa, synthetic construct
  REGION 1..10: note = Synthetic Construct
GGGLRGAMDY

SEQ ID NO: 17 - protein, 9 aa, synthetic construct
  REGION 1..9: note = Synthetic Construct
SGLRRVLDY

SEQ ID NO: 18 - protein, 9 aa, synthetic construct
  REGION 1..9: note = Synthetic Construct
KGLRYAVDY

SEQ ID NO: 19 - protein, 11 aa, synthetic construct
  REGION 1..11: note = Synthetic Construct
EGYGGYWYFDV

SEQ ID NO: 20 - protein, 12 aa, synthetic construct
  REGION 1..12: note = Synthetic Construct
EVYGNYYWYFDV

SEQ ID NO: 21 - protein, 9 aa, synthetic construct
  REGION 1..9: note = Synthetic Construct
DGYNGAMDY

SEQ ID NO: 22 - protein, 11 aa, synthetic construct
  REGION 1..11: note = Synthetic Construct
TAVMATEAMDR

SEQ ID NO: 23 - protein, 10 aa, synthetic construct
  REGION 1..10: note = Synthetic Construct
SASSSVSYMH

SEQ ID NO: 24 - protein, 10 aa, synthetic construct
  REGION 1..10: note = Synthetic Construct
SANSSVNYMY

SEQ ID NO: 25 - protein, 10 aa, synthetic construct
  REGION 1..10: note = Synthetic Construct
SASSSVSYMY

SEQ ID NO: 26 - protein, 17 aa, synthetic construct
  REGION 1..17: note = Synthetic Construct
KSSQSLLNSGNQKNYLA

SEQ ID NO: 27 - protein, 15 aa, synthetic construct
  REGION 1..15: note = Synthetic Construct
RASESVDSYGNSFMH

SEQ ID NO: 28 - protein, 15 aa, synthetic construct
  REGION 1..15: note = Synthetic Construct
RASESVDRYGNSFMH

SEQ ID NO: 29 - protein, 7 aa, synthetic construct
  REGION 1..7: note = Synthetic Construct
STSNLAS

SEQ ID NO: 30 - protein, 7 aa, synthetic construct
  REGION 1..7: note = Synthetic Construct
DTSNLAS

SEQ ID NO: 31 - protein, 7 aa, synthetic construct
  REGION 1..7: note = Synthetic Construct
GASTRES

SEQ ID NO: 32 - protein, 7 aa, synthetic construct
  REGION 1..7: note = Synthetic Construct
GTSNLAS

SEQ ID NO: 33 - protein, 7 aa, synthetic construct
  REGION 1..7: note = Synthetic Construct
RASNLES

SEQ ID NO: 34 - protein, 9 aa, synthetic construct
  REGION 1..9: note = Synthetic Construct
QQRSSYPPT

SEQ ID NO: 35 - protein, 9 aa, synthetic construct
  REGION 1..9: note = Synthetic Construct
HQRSSYPWT

SEQ ID NO: 36 - protein, 9 aa, synthetic construct
  REGION 1..9: note = Synthetic Construct
QQWSSYPRT

SEQ ID NO: 37 - protein, 9 aa, synthetic construct
  REGION 1..9: note = Synthetic Construct
QQHYSTPYT

SEQ ID NO: 38 - protein, 9 aa, synthetic construct
  REGION 1..9: note = Synthetic Construct
QQRSSYPYT

SEQ ID NO: 39 - protein, 9 aa, synthetic construct
  REGION 1..9: note = Synthetic Construct
QQSNEDPYT

SEQ ID NO: 40 - protein, 9 aa, synthetic construct
  REGION 1..9: note = Synthetic Construct
QQSNEDPLT

SEQ ID NO: 41 - protein, 118 aa, synthetic construct
  REGION 1..118: note = Synthetic Construct
EVQLQESGPDLVKPSQSLSLTCTVTGYSFTSGYSWHWIRQFPGNRLEWMGYIHDSGGTNYNPSLKSRISITRDTSKNQFFLQLNSVTTEDTATYYCAREENYRYFDVWGAGTTVTVSS

SEQ ID NO: 42 - protein, 119 aa, synthetic construct
  REGION 1..119: note = Synthetic Construct
EVQLQQSGVELARPGASVKMSCKASGYTFSSYAMQWVKKRPGQGLEWIGYINPGTGYTNYNQKFKDKATLTADKSSSTAYMHLSSLTSEDSAVYFCARGGGLRGAMDYWGQGTSVTVSS

SEQ ID NO: 43 - protein, 118 aa, synthetic construct
  REGION 1..118: note = Synthetic Construct
QIQLLQSGAELARPGASVKMSCKASGYTFTSYTMHWVKQRPGQGLEWIGYINPSSGYTNYNQKFKDKATLTADKSSSTAYMQLSSLTSEGSAVYYCARSGLRRVLDYWGQGTSVTVSS

SEQ ID NO: 44 - protein, 118 aa, synthetic construct
  REGION 1..118: note = Synthetic Construct
EVKLVESGAELARPGASVKMSCKASGYTFTSYAMHWVKQRPGQGLEWIGYINPSSGYTNYNQKFKDKATLTADKSSSTAYMQLSSLTSEDSAVYYCARKGLRYAVDYWGQGTSVTVSS

SEQ ID NO: 45 - protein, 120 aa, synthetic construct
  REGION 1..120: note = Synthetic Construct
EVKLMESGPDLVKPSQSLSLTCTVTGYSITSGYSWHWIRHFPGKKLEWMGYIHNSGSTNYNPSLKSRISISRETSKNQFFLQLNSVTTEDTATYYCAREGYGGYWYFDVWGAGTSVTVSS

SEQ ID NO: 46 - protein, 121 aa, synthetic construct
  REGION 1..121: note = Synthetic Construct
QVQLKESGPDLVKPSQSLSLTCTVTGYSITSGYSWHWIRQFPGNKLEWMTYIHSSGSTNYNPSLKSRISITRDTSKNQFFLQLKSVTTEDTATYYCAREVYGNYYWYFDVWGAGTTVTVSS

SEQ ID NO: 47 - protein, 118 aa, synthetic construct
  REGION 1..118: note = Synthetic Construct
EVQLQQSGPELVKPGASVKVSCKASGYIFTSYNMYWMKQSHGKSLEWIGYIDPYNGGTRYNQKFKGKATLTVDKSSSTAYMHLNSLTSEDSAVYYCAVDGYNGAMDYWGQGTSVTVSS

SEQ ID NO: 48 - protein, 118 aa, synthetic construct
  REGION 1..118: note = Synthetic Construct
QIQLLQSGPELVKPGASVKVSCKASGYIFTSYNMYWMKQSHGKSLEWIGYIDPYNGGTNYNQKFKGKATLTVDKSSSTAYMHLNSLTSEDSAVYYCAVDGYNGAMDYWGQGTSVTVSS

SEQ ID NO: 49 - protein, 120 aa, synthetic construct
  REGION 1..120: note = Synthetic Construct
QVQLQQPGAELVKPGASVKLSCTASGFNIKDTYMYWVKQRPEQGLEWIGRIDPANGITKFDPKFQDKATVTADTFSNIAYLHLSSLTSEDTAVYYCTTTAVMATEAMDRWGQGTSVTVSS

SEQ ID NO: 50 - protein, 118 aa, synthetic construct
  REGION 1..118: note = Synthetic Construct
QVQLQESGPGLVKPSETLSLTCAVSGYSFTSGYSWHWIRQPPGKGLEWIGYIHDSGGTNYNPSLKSRVTISRDTSKNQFSLKLSSVTAADTAVYYCAREENYRYFDVWGQGTLVTVSS

SEQ ID NO: 51 - protein, 118 aa, synthetic construct
  REGION 1..118: note = Synthetic Construct
QVQLQESGPGLVKPSETLSLTCTVTGYSFTSGYSWHWIRQPPGKGLEWIGYIHDSGGTNYNPSLKSRVTISRDTSKNQFSLKLSSVTAADTAVYYCAREENYRYFDVWGQGTLVTVSS

SEQ ID NO: 52 - protein, 106 aa, synthetic construct
  REGION 1..106: note = Synthetic Construct
DIQMMQSPAIMSASPGEKVTITCSASSSVSYMHWFQQKPGTSPKLWIYSTSNLASGVPARFSGSGSGTSYSLTISRMEAEDAATYYCQQRSSYPPTFGDGTRLEIK

SEQ ID NO: 53 - protein, 106 aa, synthetic construct
  REGION 1..106: note = Synthetic Construct
DIVLTQSPALMSASPGEKVTMTCSANSSVNYMYWYQQKPGSSPKLWIYSTSNLASGVPARFSGSGSGTSYSLTISSMEAEDAATYYCHQRSSYPWTFGGGTKLEIK

SEQ ID NO: 54 - protein, 106 aa, synthetic construct
  REGION 1..106: note = Synthetic Construct
ENVLTQSPAILSASPGEKVTMTCSASSSVSYMYWYQQKPGSSPRLLIYDTSNLASGVPVRFSGSGSGTSYSLTISRMEAEDAATYYCQQWSSYPRTFGGGTKLEIK

SEQ ID NO: 55 - protein, 113 aa, synthetic construct
  REGION 1..113: note = Synthetic Construct
DIVMTQSPSSLSVSAGEKVTMSCKSSQSLLNSGNQKNYLAWYQQKPGQPPKLLIYGASTRESGVPDRFTGSGSGTDYTLTISSVQAEDLALYYCQQHYSTPYTFGGGTKLEIK

SEQ ID NO: 56 - protein, 106 aa, synthetic construct
  REGION 1..106: note = Synthetic Construct
DIVLTQSPAIMSASPGEKVTMTCSASSSVSYMYWYQQKPGSSPKPWIYGTSNLASGVPVRFSGSGSGTSYSLTISRMEAEDAATYYCQQRSSYPYTFGGGTKLEIK

SEQ ID NO: 57 - protein, 106 aa, synthetic construct
  REGION 1..106: note = Synthetic Construct
DIVMTQSPAIMSASLGEEITLTCSASSSVSYMHWYQQKSGTSPKLLIYSTSNLASGVPARFSGSGSGTSYSLTISRMEAEDAATYYCQQRSSYPYTFGGGTKLEIK

SEQ ID NO: 58 - protein, 111 aa, synthetic construct
  REGION 1..111: note = Synthetic Construct
QIVLTQSPASLAVSLGQRATISCRASESVDSYGNSFMHWYQQKPGQPPKLLIYRASNLESGIPARFSGSGSRTDFTLTINPVEADDVATYYCQQSNEDPYTFGGGTKLEIK

SEQ ID NO: 59 - protein, 111 aa, synthetic construct
  REGION 1..111: note = Synthetic Construct
DIVLTQSPASLTVSLGQRATISCRASESVDRYGNSFMHWYQQKPGQPPKLLIYRASNLESGVPARFSGSGSRTDFTLTINPVEADDVASYYCQQSNEDPLTFGAGTKLELK

SEQ ID NO: 60 - protein, 106 aa, synthetic construct
  REGION 1..106: note = Synthetic Construct
EIVLTQSPATLSLSPGERATLSCSASSSVSYMHWFQQKPGQSPRLWIYSTSNLASGIPARFSGSGSGTDYTLTISSLEPEDFAVYYCQQRSSYPPTFGQGTKVEIK

SEQ ID NO: 61 - protein, 106 aa, synthetic construct
  REGION 1..106: note = Synthetic Construct
EIVLTQSPATLSLSPGERATLSCSASSSVSYMHWFQQKPGQSPRLLIYSTSNLASGIPARFSGSGSGTDFTLTISSLEPEDFAVYYCQQRSSYPPTFGQGTKVEIK

SEQ ID NO: 62 - protein, 330 aa, synthetic construct
  REGION 1..330: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 327 aa, synthetic construct
  REGION 1..327: note = Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 107 aa, synthetic construct [sequence omitted]

SEQ ID NO: 65 - protein, 106 aa, synthetic construct
  REGION 1..106: note = Synthetic Construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 66 - protein, 274 aa, synthetic construct
  REGION 1..274: note = Synthetic Construct
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 67 - protein, 119 aa, synthetic construct
  REGION 1..119: note = Synthetic Construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 68 - protein, 365 aa, synthetic construct
  REGION 1..365: note = Synthetic Construct
MRVPRPQPWALGLLLFLLPGSLGAESHLSLLYHLTAVSSPAPGTPAFWVSGWLGPQQYLSYDSLRGQAEPCGAWVWENQVSWYWEKETTDLRIKEKLFLEAFKALGGKGPYTLQGLLGCELSPDNTSVPTAKFALNGEEFMNFDLKQGTWGGDWPEALAISQRWQQQDKAANKELTFLLFSCPHRLREHLERGRGNLEWKEPPSMRLKARPGNPGFSVLTCSAFSFYPPELQLRFLRNGMAAGTGQGDFGPNSDGSFHASSSLTVKSGDEHHYCCIVQHAGLAQPLRVELETPAKSSVLVVGIVIGVLLLTAAAVGGALLWRRMRSGLPAPWISLRGDDTGSLLPTPGEAQDADSKDINVIPATA

SEQ ID NO: 69 - protein, 280 aa, synthetic construct
  REGION 1..280: note = Synthetic Construct
SETRPPLMYHLTAVSNPSTGLPSFWATGWLGPQQYLTYNSLRQEADPCGAWMWENQVSWYWEKETTDLKSKEQLFLEALKTLEKILNGQKRGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSS

SEQ ID NO: 70 - protein, 276 aa, synthetic construct
  REGION 1..276: note = Synthetic Construct
AEPRLPLMYHLAAVSDLSTGLPSFWATGWLGAQQYLTYNNLRQEADPCGAWIWENQVSWYWEKETTDLKSKEQLFLEAIRTLENQINGTFTLQGLLGCELAPDNSSLPTAVFALNGEEFMRFNPRTGNWSGEWPETDIVGNLWMKQPEAARKESEFLLTSCPERLLGHLERGRQNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSPARSS